(19) United States
(12) Patent Application Publication
(10) Pub. No.: US 2022/0218843 A1
(43) Pub. Date: Jul. 14, 2022

(54) NON-DISRUPTIVE GENE THERAPY FOR THE TREATMENT OF MMA

(71) Applicants: LogicBio Therapeutics, Inc., Lexington, MA (US); The United States of America, as Represented by the Secretary, Department of Health and Human Servic, Bethesda, MD (US)

(72) Inventors: Charles P. Venditti, Bethesda, MD (US); Randy J. Chandler, Bethesda, MD (US); B. Nelson Chau, Needham, MA (US); Kyle P. Chiang, Arlington, MA (US); Jing Liao, Lexington, MA (US)

(73) Assignee: LogicBio Therapeutics, Inc., Lexington, MA (US)

(21) Appl. No.: 17/267,482

(22) PCT Filed: Oct. 30, 2018
(86) PCT No.: PCT/US2018/058307

§ 371 Date: Feb. 9, 2021

Related U.S. Application Data
(60) Provisional application No. 62/717,771, filed on Aug. 10, 2018.

(51) Int. Cl. A61K 48/00; C12N 15/86; C12N 15/90; C12N 9/90

(52) U.S. Cl. CPC A61K 48/005; C12N 15/86; C12N 15/907; C12N 9/90; C12Y 504/99002; C12N 2750/14145; A01K 2227/105; A01K 2267/035; C12N 2750/14122; C12N 2750/14143; A01K 2217/075

(57) ABSTRACT

Methods and technologies for the treatment of methylmalonic acidemia.

### CROSS REFERENCE TO RELATED APPLICATIONS

This application is a U.S. National Stage Application of PCT Application No. PCT/US2018/058307, filed Oct. 30, 2018 and published as WO/2020/032986, which claims priority to U.S. Provisional Application No. 62/717,771 filed Aug. 10, 2018, the entirety of each of which is incorporated herein by reference.

### STATEMENT REGARDING FEDERALLY SPONSORED RESEARCH OR DEVELOPMENT

This invention was made in the performance of a Cooperative Research and Development Agreement with the National Institutes of Health, an Agency of the U.S. Department of Health and Human Services, and with Government support under project number ZIA HG200318 14 by the National Institutes of Health, National Human Genome Research Institute. The Government of the United States has certain rights in the invention.

### SEQUENCE LISTING

The instant application contains a Sequence Listing which has been submitted electronically in ASCII format and is hereby incorporated by reference in its entirety. Said ASCII copy, created on Oct. 30, 2018, is named 2012538_0062_SL.txt and is 78,203 bytes in size.

### BACKGROUND

There is a subset of human diseases that can be traced to changes in the DNA that are either inherited or acquired early in embryonic development. Of particular interest for developers of genetic therapies are diseases caused by a mutation in a single gene, known as monogenic diseases. There are believed to be over 6,000 monogenic diseases. Typically, any particular genetic disease caused by inherited mutations is relatively rare, but taken together, the toll of genetic-related disease is high. Well-known genetic diseases include cystic fibrosis, Duchenne muscular dystrophy, Huntington's disease and sickle cell disease. Other classes of genetic diseases include metabolic disorders, such as organic acidemias, and lysosomal storage diseases where dysfunctional genes result in defects in metabolic processes and the accumulation of toxic byproducts that can lead to serious morbidity and mortality both in the short-term and long-term.

### SUMMARY

Monogenic diseases have been of particular interest to biomedical innovators due to the perceived simplicity of their disease pathology. However, the vast majority of these diseases and disorders remain substantially untreatable. Thus, there remains a long felt need in the art for the treatment of such diseases.

In some embodiments, the present disclosure provides methods of integrating a transgene into the genome of at least a population of cells in a tissue in a subject, said methods including the step of administering to a subject in which cells in the tissue fail to express a functional protein encoded by a gene product, a composition that delivers a transgene encoding the functional protein, wherein the composition includes: a polynucleotide cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products upon integration into a target integration site in the genome of the cell, a third nucleic acid sequence positioned 5′ to the polynucleotide and comprising a sequence that is substantially homologous to a genomic sequence 5′ of the target integration site in the genome of the cell, and a fourth nucleic acid sequence positioned 3′ to the polynucleotide and comprising a sequence that is substantially homologous to a genomic sequence 3′ of the target integration site in the genome of the cell, wherein, after administering the composition, the transgene is integrated into the genome of the population of cells.

In some embodiments, the present disclosure provides methods of increasing a level of expression of a transgene in a tissue over a period of time, said methods including the step of administering to a subject in need thereof a composition that delivers a transgene that integrates into the genome of at least a population of cells in the tissue of the subject, wherein the composition includes: a polynucleotide comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products upon integration into a target integration site in the genome of the cell, a third nucleic acid sequence positioned 5′ to the polynucleotide and comprising a sequence that is substantially homologous to a genomic sequence 5′ of the target integration site in the genome of the cell, and a fourth nucleic acid sequence positioned 3′ to the polynucleotide and comprising a sequence that is substantially homologous to a genomic sequence 3′ of the target integration site in the genome of the cell, wherein, after administering the composition, the transgene is integrated into the genome of the population of cells and the level of expression of the transgene in the tissue increases over a period of time. In some embodiments, the increased level of expression comprises an increased percent of cells in the tissue expressing the transgene.

In some embodiments, the present disclosure provides methods including a step of administering to a subject a dose of a composition that delivers to cells in a tissue of the subject a transgene encoding a product of interest that is not functionally expressed by the cells prior to the administering, wherein the transgene (i) encodes the product of interest; (ii) integrates at a target integration site in the genome of a plurality of the cells; (iii) functionally expresses the product of interest once integrated; and (iv) confers a selective advantage to the plurality of cells relative to other cells in the tissue, so that, over time, the tissue achieves a level of functional expression of the product of interest that has been determined to be higher than that achieved by otherwise comparable administering wherein the cells in which the transgene is integrated do functionally express the product of interest prior to the administering, wherein the composition comprises: a polynucleotide comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products when the transgene is integrated at the target integration site, a third nucleic acid sequence positioned 5′ to the polynucleotide and comprising a sequence that is substantially homologous to a genomic sequence 5′ of the target integration site, and a fourth nucleic acid sequence positioned 3′ to the polynucleotide and comprising a sequence that is substantially homologous to a genomic sequence 3′ of the target integration site. In some embodiments, the selective advantage comprises an increased percent of cells in the tissue expressing the transgene.

In some embodiments, a composition comprises a recombinant viral vector. In some embodiments, a recombinant viral vector is a recombinant AAV vector. In some embodiments, a recombinant viral vector is or comprises a capsid protein comprising an amino acid sequence having at least 95% sequence identity with the amino acid sequence of LK03, AAV8, AAV-DJ; AAV-LK03; or AAVNP59. In some embodiments, the composition further comprises AAV2 ITR sequences.

In accordance with various embodiments, any of a variety of transgenes may be expressed in accordance with the methods and compositions described herein. For example, in some embodiments, a transgene is or comprises a MUT transgene. In some embodiments, a MUT transgene is a wt human MUT; a codon optimized MUT; a synthetic MUT; a MUT variant; a MUT mutant, or a MUT fragment.

In some embodiments, the present invention provides recombinant viral vectors for integrating a transgene into a target integration site in the genome of a cell, including: a polynucleotide cassette comprising a first nucleic acid sequence and a second nucleic acid sequence, wherein the first nucleic acid sequence comprises a MUT transgene; and the second nucleic acid sequence is positioned 5′ or 3′ to the first nucleic acid sequence and promotes the production of two independent gene products upon integration into the target integration site in the genome of the cell, a third nucleic acid sequence positioned 5′ to the polynucleotide cassette vector and comprising a sequence that is substantially homologous to a genomic sequence 5′ of the target integration site in the genome of the cell, and a fourth nucleic acid sequence positioned 3′ of the polynucleotide cassette viral vector and comprising a sequence that is substantially homologous to a genomic sequence 3′ of the target integration site in the genome of the cell, wherein the viral vector comprises an LK03 AAV capsid.

As is described herein, the present disclosure encompasses several advantageous recognitions regarding the integration of one or more transgenes into the genome of a cell. For example, in some embodiments, integration does not comprise nuclease activity.

While any application-appropriate tissue may be targeted, in some embodiments, the tissue is the liver.

As is described herein, provided methods and compositions include polynucleotide cassettes with at least four nucleic acid sequences. In some embodiments, the second nucleic acid sequence comprises: a) a 2A peptide, b) an internal ribosome entry site (IRES), c) an N-terminal intein splicing region and C-terminal intein splicing region, or d) a splice donor and a splice acceptor. In some embodiments, the third and fourth nucleic acid sequences are homology arms that integrate the transgene and the second nucleic acid sequence into an endogenous albumin gene locus comprising an endogenous albumin promoter and an endogenous albumin gene. In some embodiments, the homology arms direct integration of the polynucleotide cassette immediately 3′ of the start codon of the endogenous albumin gene or immediately 5′ of the stop codon of the endogenous albumin gene.

In accordance with various aspects, the third and/or fourth nucleic acids may be of significant length (e.g., at least 800 nucleotides in length). In some embodiments, the third nucleic acid is between 800-1,200 nucleotides. In some embodiments, the fourth nucleic acid is between 800-1,200 nucleotides.

In some embodiments, the polynucleotide cassette does not comprise a promoter sequence. In some embodiments, upon integration of the polynucleotide cassette into the target integration site in the genome of the cell, the transgene is expressed under control of an endogenous promoter at the target integration site. In some embodiments, the target integration site is an albumin locus comprising an endogenous albumin promoter and an endogenous albumin gene. In some embodiments, upon integration of the polynucleotide cassette into the target integration site in the genome of a cell, the transgene is expressed under control of the endogenous albumin promoter without disruption of the endogenous albumin gene expression.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

FIG. 1 depicts the homology directed repair (HDR) and non-homologous end joining (NHEJ) DNA repair pathways.

FIG. 2 shows a schematic of the GENERIDE™ construct before integration (AAV) and following HR-mediated integration into the genome at the targeted Albumin, or ALB, locus. Expression from the targeted locus results in the production of albumin and transgene, as separate proteins, at equivalent levels, which is coded for by the ALB gene.

FIG. 3 shows the most abundant genes expressed in the liver, ranked from highest (ALB) to number 2,000. Each circle represents an individual gene. Most genes in the liver are expressed at a small fraction of the levels of albumin. TPM=transcripts per million.

FIG. 4 shows that the liver is the organ where nearly all albumin is expressed in the body. Liver-specific GENERIDE™ constructs targeting the ALB locus will predominantly be expressed in the liver.

FIG. 5 shows that albumin expression levels are 100× higher than other select liver genes associated with monogenic diseases. (PAH: phenylketonuria, F9: hemophilia B, MUT: MMA, UGT1A1: Crigler-Najjar syndrome).

FIG. 6 illustrates how mutations in MUT result in a disorder of the metabolic pathway for branched chain amino acids, specifically methionine, threonine, valine and isoleucine.

FIG. 7A-FIG. 7B illustrate the structure of LB-001 GENERIDE™ construct. FIG. 7A) The GENERIDE™ construct for LB-001 inside an LK03 AAV capsid. FIG. 7B) A nucleic acid that can be used with the AAV-LK03 capsid to express a human Mut sequence (SEQ ID NO: 15).

FIG. 8 shows that Mut−/− mice display enhanced survival (upper panel) and weight gain (lower panel) following neonatal treatment with a murine GENERIDE™ construct of LB-001. Error bars indicate standard error of the mean, or SEM. Control mice were not included as a head-to-head comparator in this study; control mouse data is derived from studies completed by others.

FIG. 9 shows that MCK-Mut mice treated with a murine GENERIDE™ construct of LB-001 show significant improvement in growth at one month following a neonatal administration. * indicates p-value<0.05.

FIG. 10 shows that MCK-Mut mice treated with a murine GENERIDE™ construct of LB-001 show significant reduction of two circulating disease related metabolites at one month, following a neonatal administration. Upper panel shows the reduction in plasma methylmalonic acid concentrations. Lower panel shows the reduction in plasma methylcitrate concentrations. Not all untreated mice were included as a head-to-head comparator. Untreated mouse data includes historical control mice. * indicates p-value<0.05.

FIG. 11 shows that treatment with GENERIDE™ can result in a selective advantage to modified liver cells. Upper panel: RNAscope analysis of liver sections from mice treated with a murine GENERIDE™ construct of LB-001. Mice genetically engineered without (left) and with (right) a functioning copy of Mut in the liver were treated neonatally. After more than one year, cells expressing the Mut mRNA specific to the GENERIDE™ construct (dark staining regions) were increased in the mice lacking a natural functioning copy of Mut in the liver, suggestive of a beneficial selective advantage of the GENERIDE™ construct of LB-001. Lower panel: quantitation of RNAscope sections conducted by an independent pathologist.

FIG. 12 shows percent of liver cells containing an integrated copy of the GENERIDE™ specific Mut gene more than one year after a single neonatal administration of a MUT GENERIDE™ construct in mice. LR-qPCR quantitation of DNA with the Mut gene integrated at the albumin locus. Error bars indicate SEM. LR-qPCR=long-range quantitative PCR.

FIG. 13 demonstrates an increase in cells with integrated GENERIDE™ construct observed over time. Mice deficient in liver Mut were administered a GENERIDE™ construct as neonates. DNA analysis for integration at the albumin locus was conducted by LR-qPCR at 1 month and more than one-year post dose. Error bars indicate SEM.

FIG. 14 Plasma methylmalonic acid levels in untreated and treated Mut^{-/-}; Mck-Mut mice (hypomorphic model of MMA). Treated mice had significantly reduced plasma methylmalonic acid levels compared to untreated mice at 1, 2 and 12-15 months post-treatment (unpaired t-test; p>0.041). The plasma methylmalonic acids levels decreased over time in the treated Mut^{-/-}; Mck-Mut animals.

FIG. 15A-FIG. 15B shows viral genomes and hepatocyte transgene integrations after delivery. FIG. 15A) The number of viral genomes (MUT) relative to host genomes (Gapdh) detected by digital droplet PCR in the liver at 1 month (n=3); 2 months (n=3); and 12-15 months (n=5) post-injection. A rapid loss of viral genomes occurs after neonatal gene delivery, which has been previously described. (Viral genomes at 1 month versus 2 or 12-15 months; one-way ANOVA; p>0.001). FIG. 15B) The percent of hepatocytes with transgene integrations into Albumin. The percentage of integrations determined by qPCR was significantly increases from 1-2 months (n=6) to 12-15 months (n=5) in the treated MMA mice (unpaired t test; p>0.043). However, at 12-15 months treated wild-type animals have less integrations than treated MMA mice.

FIG. 16 shows hepatic MUT protein expression in treated mice. Total hepatic MUT protein expression in AAV-Alb-2A-MUT treated mice was determined by western blot. MUT protein in treated mice is expressed as a percentage of a wild-type control littermate and was normalized to murine beta-actin. The amount of MUT protein in treated mice increases over time when comparing 1-2 month (n=6) to 12-15 months (n=5) post-treatment (unpaired t-test; p>0.015).

FIG. 17 shows RNAscope of AAV-Alb-2A-MUT treated mice to detect MUT mRNA positive cells. There is an increase in MUT positive cells in mice 12-15 months post-treatment when compared to 2 months post-treatment. Conversely, AAV-Alb-2A-MUT treated wild-type mice 12-15 months post-treatment (n=5) have fewer MUT positive cells than their MMA littermates at 12-15 months post-treatment (n=5) (p>0.03).

FIG. 18A-FIG. 18B. show the percent gDNA integration determined with LR-qPCR assay after the listed doses of a murine LB001 surrogate were administered IV via facial vein on 1 day after birth. Liver samples were harvested at indicated timepoints. FIG. 18A) Shows data for Mut^{-/-}; Mck⁺ mice. FIG. 18B) Shows data for heterozygote Mut^{+/-} mice.

FIG. 19 Fused mRNA from primary human hepatocytes. Exons 12 and 15 are outside of the homology arms. The figure discloses SEQ ID NOs: 17-19, respectfully, in order of appearance.

FIG. 20 depicts a primary human hepatocyte sandwich culture system.

FIG. 21A-FIG. 21B illustrates an assay for DNA integration. FIG. 21A) A stable HepG2-2A-PuroR cell line was used as a positive control in the DNA integration assay. FIG. 21B) Long-range (LR) qPCR was used to determine site-specific integration rate.

FIG. 22 shows relative expression of MUT and ALB in primary human hepatocytes (PHH).

FIG. 23A-FIG. 23B shows three primary human hepatocyte (PHH) donors with the same haplotype 1 that were chosen to assay GENERIDE™ LB-001. FIG. 23A) Haplotype screening from 22 PHH donors. FIG. 23B) Haplotype information.

FIG. 24 shows optimization of transduction conditions of primary human hepatocytes (PHH) using AAV-LK03-LSP-GFP. Transduction efficiency is shown in PHH from three selected donors.

FIG. 25 depicts Western blotting result of ALB-2a and MUT expression after GENERIDE™ LB001 treatment in primary human hepatocyte (PHH).

FIG. 26 shows increased survival in a mouse model of Crigler-Najjar syndrome following neonatal administration of a GENERIDE™ construct delivering UGT1A1 (Porro et al. EMBO Mol Med 2017). Untreated animals (n=6) all died within 20 days of birth without continued blue-light therapy. Blue-light therapy, a treatment that facilitates clearance and reduction of toxic bilirubin levels, was applied from birth to Day 8. Without continued blue-light therapy, animals treated with a GENERIDE™ construct (n=5) survived for one year.

FIG. 27 Therapeutic and stable levels of human factor IX with a murine GENERIDE™ construct of LB-101 (Barzel et al. Nature 2015). Stable and therapeutic levels of factor IX production from the liver, following neonatal administration, persisted for 20 weeks after administration, even with a PH conducted at 8 weeks of age (therapeutic levels of factor IX between 5% and 20% of normal factor IX shown by dashed lines and the shaded region). Error bars indicate standard deviation.

FIG. 28 shows amelioration of the bleeding diathesis in hemophilia B mice using a GENERIDE™™ vector coding a hyper-active hFIX. Measurement of coagulation efficiency by activated partial thromboplastin time (aPTT) 2 weeks after tail vein injections of AAV-DJ-hFIX variant (V-hFIX) compared to AAV-DJ-WThFIX, Vehicle and relative to wild-type (WT), to 9 weeks old male hemophilia B (FIX-KO) mice at the designated doses. The triangle represents the difference between AAV-DJ-V-hFIX and WT-hFIX at the same dose. Error bars represent standard deviation. *p<0.01, **p<0.001.

FIG. 29 shows amelioration of the bleeding diathesis in hemophilia B neonatal mice using a GENERIDE™™ vector coding a proprietary hyper-active hFIX. Measurement of coagulation efficiency by activated partial thromboplastin time (aPTT) 4 weeks (left panel) and 12 weeks (right panel) weeks after Intraperitoneal (IP) injections of AAV-V-hFIX compared to Vehicle and relative to WT reference. For the treatment of hemophilia B neonatal mice, we performed Intraperitoneal (IP) injections of 2-day old F9tm1Dws knockout male mice with 1.5e14, 1.5e13, 1.5e12 and 5e11 vector genomes (vg) per kilogram (kg) of a AAV-DJ GENERIDE™™ vector coding for a hFIX variant. We demonstrated disease amelioration at doses as low as 1.5e12 vg/kg. The functional coagulation, as determined by the activated partial thromboplastin time (aPTT) in treated KO male mice, was restored to levels similar to that of wild-type (WT) mice. Error bars represent standard deviation. *p<0.01, **p<0.001.

FIG. 30A-FIG. 30C shows that GENERIDE™ remains effective with mismatched homology arms. Depicted are two major haplotypes in the human albumin locus. The haplotypes differ by 5 SNPs in the sequence corresponding to the 5′ homology arm. FIG. 30A) A segment of the human albumin locus spanning the stop codon is depicted as a horizontal thin rectangle. Short longitudinal lines represent the relative position of nucleotide polymorphisms between the two most common haplotypes in the human population, haplotype 1 and haplotype 2. 95% of albumin alleles in the human population are evenly distributed, at the relevant segment, between these two haplotypes, differing by only 6 nucleotides. The specific nucleotides at the polymorphic positions in haplotypes 1 and 2 are presented above and below the line, respectively. FIG. 30B) Depicted are two GENERIDE™™ AAV vectors targeting the proprietary human FIX variant (V-hFIX) into the mouse albumin locus. The homology arms in the upper vector “wild-type arms (WTA)” are identical to the genomic sequences spanning the albumin stop codon in B6 mice. The homology arms in the bottom vector “mismatched arm (MA)” differ from the WT arms in a manner that simulates the difference between the human haplotypes: haplotype 1 and haplotype 2. The short longitudinal lines represent the relative position of nucleotide polymorphisms between the two vectors. The specific nucleotides at the polymorphic positions in the two vectors are presented above each line. FIG. 30C) hFIX plasma measured by ELISA following tail vein injections of 9-week-old B6 mice with 5e13 per vg/kg of either the AAV V-hFIX-WTA experimental construct (n=5), or haplotype mismatched AAV V-hFIX-MA from three independent batches (n=5/group). Error bars represent standard deviation.

FIG. 31A-FIG. 31B depict murine models of MMA. FIG. 31A) Mut^{-/-} mouse model with Mut exon 3 knock-out. This mouse is neonatal lethal. Previously presented in Chandler et al. BMC Med Genet. 2007. FIG. 31B) Mut^{-/-}Mck⁺ mouse model. This mouse model has muscle specific Mut expression and the mice are viable.

FIG. 32 depicts experimental designs for analysis of MMA mouse models after administration of GENERIDE™ constructs.

### DETAILED DESCRIPTION

### Gene Therapy

Gene therapies alter the gene expression profile of a patient's cells by gene transfer, a process of delivering a therapeutic gene, called a transgene. Drug developers use modified viruses as vectors to transport transgenes into the nucleus of a cell to alter or augment the cell's capabilities. Developers have made great strides in introducing genes into cells in tissues such as the liver, the retina of the eye and the blood-forming cells of the bone marrow using a variety of vectors. These approaches have in some cases led to approved therapies and, in other cases, have shown very promising results in clinical trials.

There are multiple gene therapy approaches. In conventional AAV gene therapy, the transgene is introduced into the nucleus of the host cell, but is not intended to integrate in chromosomal DNA. The transgene is expressed from a non-integrated genetic element called an episome that exists inside the nucleus. A second type of gene therapy employs the use of a different type of virus, such as lentivirus, that inserts itself, along with the transgene, into the chromosomal DNA but at arbitrary sites.

Episomal expression of a gene must be driven by an exogenous promoter, leading to production of a protein that corrects or ameliorates the disease condition.

### Limitations of Gene Therapy

Dilution effects as cells divide and tissues grow. In the case of gene therapy based on episomal expression, when cells divide during the process of growth or tissue regeneration, the benefits of the therapy typically decline because the transgenes were not intended to integrate into the host chromosome, thus not replicated during cell division. Each new generation of cells thus further reduces the proportion of cells expressing the transgene in the target tissue, leading to the reduction or elimination of the therapeutic benefit over time.

Inability to control site of insertion. While the use of some gene therapy using viral mediated insertion has the potential to provide long-term benefit because the gene is inserted into the host chromosome, there is no ability to control where the gene is inserted, which presents a risk of disrupting an essential gene or inserting into a location that can promote undesired effects such as tumor formation. For this reason, these integrating gene therapy approaches are primarily limited to ex vivo approaches, where the cells are treated outside the body and then re-inserted.

Use of exogenous promoters increases the risk of tumor formation. A common feature of both gene therapy approaches is that the transgene is introduced into cells together with an exogenous promoter. Promoters are required to initiate the transcription and amplification of DNA to messenger RNA, or mRNA, which will ultimately be translated into protein. Expression of high levels of therapeutic proteins from a gene therapy transgene requires strong, engineered promoters. While these promoters are essential for protein expression, previous studies conducted by others in animal models have shown that non-specific integration of gene therapy vectors can result in significant increases in the development of tumors. The strength of the promoters plays a crucial role in the increase of the development of these tumors. Thus, attempts to drive high levels of expression with strong promoters may have long-term deleterious consequences.

### Gene Editing

Gene editing is the deletion, alteration or augmentation of aberrant genes by introducing breaks in the DNA of cells using exogenously delivered gene editing mechanisms. Most current gene editing approaches have been limited in their efficacy due to high rates of unwanted on- and off-target modifications and low efficiency of gene correction, resulting in part from the cell trying to rapidly repair the introduced DNA break. The current focus of gene editing is on disabling a dysfunctional gene or correcting or skipping an individual deleterious mutation within a gene. Due to the number of possible mutations, neither of these approaches can address the entire population of mutations within a particular genetic disease, as would be addressed by the insertion of a full corrective gene.

Unlike the gene therapy approach, gene editing allows for the repaired genetic region to propagate to new generations of cells through normal cell division. Furthermore, the desired protein can be expressed using the cell's own regulatory machinery. The traditional approach to gene editing is nuclease-based, and it uses nuclease enzymes derived from bacteria to cut the DNA at a specific place in order to cause a deletion, make an alteration or apply a corrective sequence to the body's DNA.

Once nucleases have cut the DNA, traditional gene editing techniques modify DNA using two routes: homology-directed repair, or HDR and non-homologous end joining, or NHEJ. HDR involves highly precise incorporation of correct DNA sequences complementary to a site of DNA damage. HDR has key advantages in that it can repair DNA with high fidelity and it avoids the introduction of unwanted mutations at the site of correction. NHEJ is a less selective, more error-prone process that rapidly joins the ends of broken DNA, resulting in a high frequency of insertions or deletions at the break site.

### Nuclease-Based Gene Editing

Nuclease-based gene editing uses nucleases, enzymes that were engineered or initially identified in bacteria that cut DNA. Nuclease-based gene editing is a two-step process. First, an exogenous nuclease, which is capable of cutting one or both strands in the double-stranded DNA, is directed to the desired site by a synthetic guide RNA and makes a specific cut. After the nuclease makes the desired cut or cuts, the cell's DNA repair machinery is activated and completes the editing process through either NHEJ or, less commonly, HDR.

NHEJ can occur in the absence of a DNA template for the cell to copy as it repairs a DNA cut. This is the primary or default pathway that the cell uses to repair double-stranded breaks. The NHEJ mechanism can be used to introduce small insertions or deletions, known as indels, resulting in the knocking out of the function of the gene. NHEJ creates insertions and deletions in the DNA due to its mode of repair and can also result in the introduction of off-target, unwanted mutations including chromosomal aberrations.

Nuclease-mediated HDR occurs with the co-delivery of the nuclease, a guide RNA and a DNA template that is similar to the DNA that has been cut. Consequently, the cell can use this template to construct reparative DNA, resulting in the replacement of defective genetic sequences with correct ones. We believe the HDR mechanism is the preferred repair pathway when using a nuclease-based approach to insert a corrective sequence due to its high fidelity. However, a majority of the repair to the genome after being cut with a nuclease continues to use the NHEJ mechanism. The more frequent NHEJ repair pathway has the potential to cause unwanted mutations at the cut site, thus limiting the range of diseases that any nuclease-based gene editing approaches can target at this time.

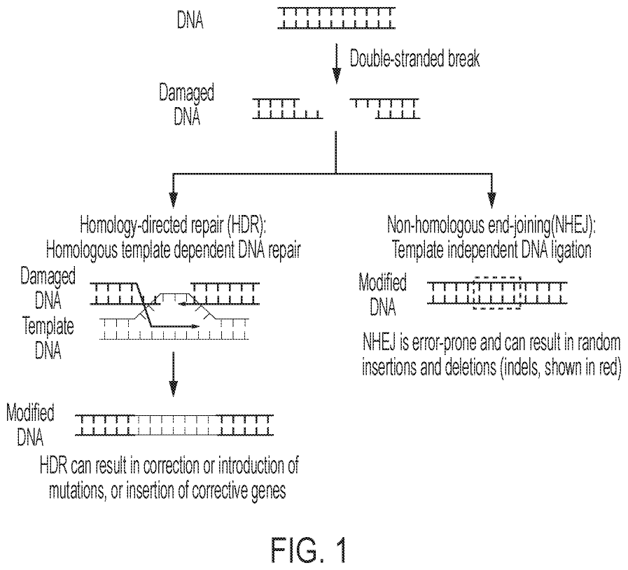

The homology-directed and non-homologous end-joining DNA repair pathways used for genome editing are illustrated in FIG. 1.

Traditional gene editing has used one of three nuclease-based approaches: Transcription activator-like effector nucleases, or TALENs; Clustered, Regularly Interspaced Short Palindromic Repeats Associated protein-9, or CRISPR/Cas9; and Zinc Finger Nucleases, or ZFN. While these approaches have already contributed to significant advances in research and product development, we believe they have inherent limitations.

### Limitations of Nuclease-Based Gene Editing

Nuclease-based gene editing approaches are limited by their use of bacterial nuclease enzymes to cut DNA and by their reliance on exogenous promoters for transgene expression. These limitations include:

Nucleases cause on- and off-target mutations. Conventional gene editing technologies can result in genotoxicity, including chromosomal alterations, based on the error-prone NHEJ process and potential off-target nuclease activity.

Delivery of gene editing components to cells is complex. Gene editing requires multiple components to be delivered into the same cell at the same time. This is technically challenging and currently requires the use of multiple vectors.

Bacterially derived nucleases are immunogenic. Because the nucleases used in conventional gene editing approaches are mostly bacterially derived, they have a higher potential for immunogenicity, which in turn limits their utility.

Because of these limitations, gene editing has been primarily restricted to ex vivo applications in cells, such as hematopoietic cells.

### GENERIDE™ Technology Platform

GENERIDE™ is a genome editing technology that harnesses homologous recombination, or HR, a naturally occurring DNA repair process that maintains the fidelity of the genome. By using HR, GENERIDE™ allows insertion of therapeutic genes, known as transgenes, into specific targeted genomic locations without using exogenous nucleases, which are enzymes engineered to cut DNA. GENERIDE™-directed transgene integration is designed to leverage endogenous promoters at these targeted locations to drive high levels of tissue-specific gene expression, without the detrimental issues that have been associated with the use of exogenous promoters.

GENERIDE™ technology is designed to precisely integrate corrective genes into a patient's genome to provide a stable therapeutic effect. Because GENERIDE™ is designed to have this durable therapeutic effect, it can be applied to targeting rare liver disorders in pediatric patients where it is critical to provide treatment early in a patient's life before irreversible disease pathology can occur. Exemplary product candidate, LB-001, is described herein for the treatment of Methylmalonic Acidemia, or MMA, a life-threatening disease that presents at birth.

GENERIDE™ platform technology has the potential to overcome some of the key limitations of both traditional gene therapy and conventional gene editing approaches in a way that is well-positioned to treat genetic diseases, particularly in pediatric patients. GENERIDE™ uses an AAV vector to deliver a gene into the nucleus of the cell. It then uses HR to stably integrate the corrective gene into the genome of the recipient at a location where it is regulated by an endogenous promoter, leading to the potential for lifelong protein production, even as the body grows and changes over time, which is not feasible with conventional AAV gene therapy.

GENERIDE™ offers several key advantages over gene therapy and gene editing technologies that rely on exogenous promoters and nucleases. By harnessing the naturally occurring process of HR, GENERIDE™ does not face the same challenges associated with gene editing approaches that rely on engineered bacterial nuclease enzymes. The use of these enzymes has been associated with significantly increased risk of unwanted and potentially dangerous modifications in the host cell's DNA, which can lead to an increased risk of tumor formation. Furthermore, in contrast to conventional gene therapy, GENERIDE™ is intended to provide precise, site-specific, stable and durable integration of a corrective gene into the chromosome of a host cell. In preclinical animal studies with GENERIDE™ constructs, integration of the corrective gene in a specific location in the genome is observed. This gives it the potential to provide a more durable approach than gene therapy technologies that do not integrate into the genome and lose their effect as cells divide. These benefits make GENERIDE™ well-positioned to treat genetic diseases, particularly in pediatric patients.

The modular approach disclosed herein can be applied to allow GENERIDE™ to deliver robust, tissue-specific gene expression that will be reproducible across different therapeutics delivered to the same tissue. By substituting a different transgene within the GENERIDE™ construct, that transgene can be delivered to address a new therapeutic indication while substantially maintaining all other components of the construct. This approach will allow leverage of common manufacturing processes and analytics across different GENERIDE™ product candidates and could shorten the development process of other treatment programs.

Previous work on non-disruptive gene targeting is described in WO 2013/158309, incorporated herein by reference. Previous work on genome editing without nucleases is described in WO 2015/143177, incorporated herein by reference.

### Genome Editing Using GENERIDE™: Mechanism and Attributes

Genome editing with the GENERIDE™ platform differs from gene editing because it uses HR to deliver the corrective gene to one specific location in the genome. GENERIDE™ inserts the corrective gene in a precise manner, leading to site-specific integration in the genome. The GENERIDE™ genome editing approach does not require the use of exogenous nucleases or promoters; instead, it leverages the cell's existing machinery to integrate and initiate transcription of therapeutic transgenes.

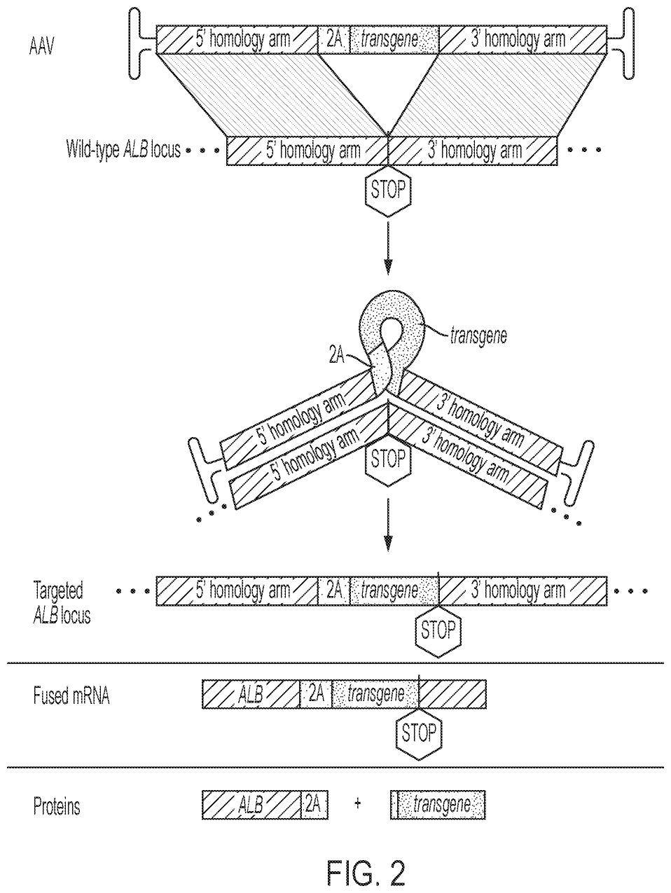

FIG. 2 shows how a GENERIDE™ construct inserts a transgene at a specific point next to the albumin gene using HR.

The GENERIDE™ technology consists of three fundamental components, each of which contributes to the potential benefits of the GENERIDE™ approach:

Homology arms comprised of hundreds of nucleotides. Flanking sequences, known as homology arms, direct site-specific integration and limit off-target insertion of the construct. Each arm is hundreds of nucleotides long, in contrast to guide sequences used in CRISPR/Cas9, which are only dozens of base pairs long, and this increased length may promote improved precision and site-specific integration. GENERIDE™'s homology arms direct the integration of the transgene immediately behind a highly expressed gene, which is observed in animal models to result in high levels of expression without the need to introduce an exogenous promoter.

Transgene. Corrective genes, known as transgenes, are chosen to integrate into the host cell's genome. These transgenes are the functional versions of the disease associated genes found in a patient's cells. The combined size of the transgenes and the homology arms can be optimized to increase the likelihood that these transgenes are of a suitable sequence length to be efficiently packaged in a capsid, which can increase the likelihood that the transgenes will ultimately be delivered appropriately in the patient.

2A peptide for polycistronic expression. A short sequence coding for a 2A peptide plays a number of important roles. First, the 2A peptide facilitates polycistronic expression, which is the production of two distinct proteins from the same mRNA. This, in turn, allows integration of a transgene in a non-disruptive way by coupling transcription of the transgene to a highly expressed target gene in the tissue of interest, driven by a strong endogenous promoter. For liver-directed therapeutic programs, including LB-001, the albumin locus can function as the site of integration. Through a process known as ribosomal skipping, the 2A peptide facilitates production of the therapeutic protein at the same level as albumin in each modified cell. Second, the patient's albumin is produced normally, except for the addition of a C-terminal tag that serves as a circulating biomarker to indicate successful integration and expression of the transgene. This modification to albumin will have minimal effect on its function, based on the results of clinical trials of other albumin protein fusions. The 2A peptide has been incorporated into other potential therapeutics such as T cell receptor chimeric antigen receptors, or CAR-Ts (Qasim et al. Sci Transl Med 2017).

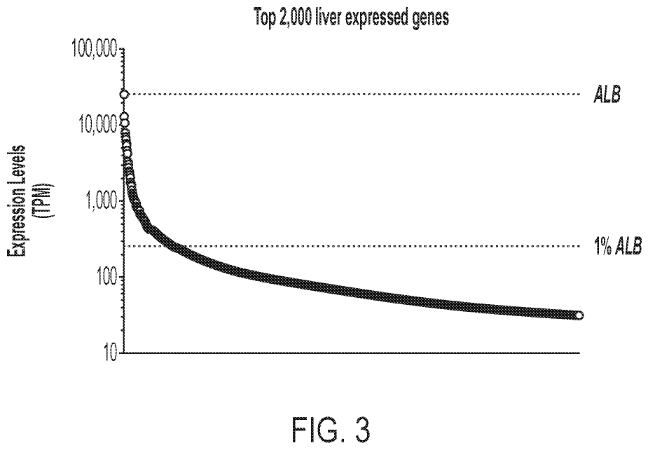

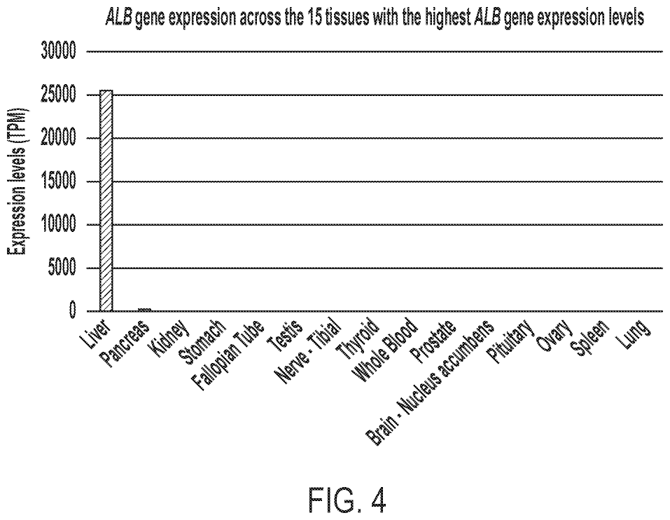

A key step in applying the GENERIDE™ platform is to identify the target genetic locus for integration. This is important because the location will dictate regulation of transgene expression, specifically the levels and tissues where the protein will be produced. For liver-directed therapeutic programs, including LB-001, the albumin locus can be used as the site of integration (see FIG. 3 and FIG. 4).

Targeting the albumin locus allows leverage of the strong endogenous promotor that drives the high level of albumin production to maximize the expression of a transgene. Linking expression of the transgene to albumin can allow expression of the transgene at therapeutic levels without requiring the addition of exogenous promoters or the integration of the transgene in a majority of target cells.

This is supported by animal models of MMA, hemophilia B and Crigler-Najjar syndrome. In these models, integration of the transgene into approximately 1% of cells resulted in therapeutic benefit. The strength of the albumin promoter overcomes the modest levels of integration to yield potentially therapeutic levels of transgene expression.

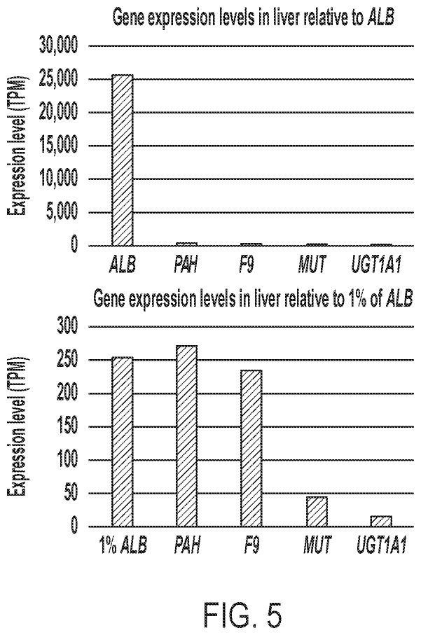

FIG. 5 shows the relative expression levels of albumin as compared to select disease-related genes in the liver, including methylmalonyl-CoA mutase, or MUT, the deficient gene in patients with MMA.

GENERIDE™ leads to integration of the corrective gene at the albumin locus in preclinical mouse models of disease, non-human primates and human cells (in vitro). In addition, the efficiency of HR that is required for transgene expression with GENERIDE™ is enhanced at sites of active transcription and is likely to be low in tissue where albumin is not actively expressed. This feature should make both on-target and off-target integration a more predictable process across programs that use the albumin locus for integration. In addition, because the GENERIDE™ platform uses HR, GENERIDE™ product candidates do not contain any bacterial nucleases, addressing the risk of on-target or off-target integration into other sites that are associated with bacterial nucleases. The GENERIDE™ therapeutic approach may be applied to other tissues and target locations in the genome. In in vitro feasibility studies, GENERIDE™ has been amenable to integration at other genomic loci, including rDNA, LAMA3 and COL7A1.

Potential advantages of the GENERIDE™ approach include the following:

Targeted integration of transgene into the genome. Conventional gene therapy approaches deliver therapeutic transgenes to target cells. A major shortcoming with most of these approaches is that once the genes are inside the cell, they do not integrate into the host cell's chromosomes and do not benefit from the natural processes that lead to replication and segregation of DNA during cell division. This is particularly problematic when conventional gene therapies are introduced early in the patient's life, because the rapid growth of tissues during the child's normal development will result in dilution and eventual loss of the therapeutic benefit associated with the transgene. Non-integrated genes expressed outside the genome on a separate strand of DNA are called episomes. This episomal expression can be effective in the initial cells that are transduced, some of which may last for a long time or for the life of a patient. However, episomal expression is typically transient in target tissues such as the liver, in which there is high turnover of cells and which tends to grow considerably in size during the course of a pediatric patient's life. With GENERIDE™ technology, the transgene is integrated into the genome, which has the potential to provide stable and durable transgene expression as the cells divide and the tissue of the patient grows, and may result in a durable therapeutic benefit.

Transgene expression without exogenous promoters. With GENERIDE™ technology, the transgene is expressed at a location where it is regulated by a potent endogenous promoter. Specifically, long homology arms can be used to insert the transgene at a precise site in the genome that is expressed under the control of a potent endogenous promoter, like the albumin promoter. By not using exogenous promoters to drive expression of a transgene, this technology avoids the potential for off-target integration of promoters, which has been associated with an increased risk of cancer. The choice of strong endogenous promoters will allow reaching therapeutic levels of protein expression from the transgene with the modest integration rates typical of the highly accurate and reliable process of HR. Accurate insertion of the transgene and the resulting expression by the cells in animal models in vivo and human cells in vitro has been observed with the GENERIDE™ technology.

Nuclease-free genome editing. By harnessing the naturally occurring process of HR, GENERIDE™ is designed to avoid undesired side effects associated with exogenous nucleases used in conventional gene editing technologies. The use of these engineered enzymes has been associated with genotoxicity, including chromosomal alterations, resulting from the error-prone DNA repair of double-stranded DNA cuts. Avoiding the use of nucleases also reduces the number of exogenous components needed to be delivered to the cell.

Modularity. A modular approach will allow GENERIDE™ to deliver robust, tissue-specific gene expression that will be reproducible across different therapeutics targeting the same tissue. The AAV capsid serves as the vehicle that enables delivery of the rest of the components to cells in the body. Vectors can be designed to be highly efficient in delivering their contents to specific target tissues such as the liver. The homology arms, which are independent of the transgene, are segments of DNA that each are hundreds of bases long and direct the integration of the target gene to a precise location in the genome. This location is critical because it determines which endogenous promoter will express the transgene. For example, a new therapy based on liver expression of a transgene could use the same capsid and homology arms as LB-001 with the transgene for the new therapy replacing the MUT gene from LB-001. By substituting a different transgene within the GENERIDE™ construct, that transgene can be delivered to address a new therapeutic indication while substantially maintaining all other components of the construct. This approach will allow leverage of common manufacturing processes and analytics across future GENERIDE™ product candidates and could potentially shorten the development process of future programs.

### MMA

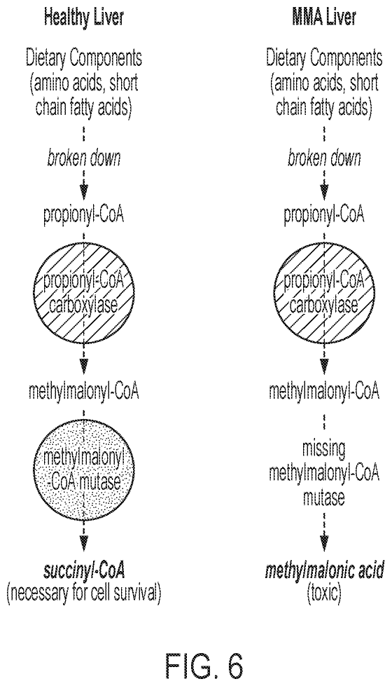

MMA can be caused by mutations in several genes which encode enzymes responsible for the normal metabolism of certain amino acids. The most common mutations are in the gene for MUT, which cause complete or partial deficiencies in its activity. As a result, a substance called methylmalonic acid and other potentially toxic compounds can accumulate, causing the signs and symptoms of MMA. FIG. 6 illustrates the effect of MUT deficiency in liver cells.

Patients with MMA suffer from frequent, and potentially lethal, episodes of metabolic instability, which accounts for the severe morbidity and early mortality observed. The effects of MMA usually appear in early infancy, with symptoms including lethargy, vomiting, dehydration and failure to thrive. Patients with MMA have long-term complications including feeding problems, intellectual disability, kidney disease and pancreatitis. Without treatment, MMA leads to coma and death. There are currently no approved therapies for MMA and the outlook for MMA patients remains poor. Management of the disease is limited to a low-protein, high-calorie diet, lacking amino acids normally processed by the MUT pathway. Despite dietary management and vigilant care, MMA patients, especially those with the most severe deficiencies in MUT, often suffer neurologic and kidney damage exacerbated during periods of catabolic stress when injury, infection or illness trigger the breakdown of protein in the body. Life expectancy for patients with MMA has increased over the past few decades, but is still estimated to be limited to approximately 20 to 30 years. Quality of life for both patients and their families and caregivers is significantly impacted by the disease due to the constraints it places on school life and social functioning. Early intervention in this vulnerable population is essential to combat the manifestation of irreversible clinical disease pathologies.

The incidence of MMA in the United States is reported to be 1 in 50,000 births, with a current prevalence of approximately 1,600 to 2,400 patients in the United States. The proportion of MMA patients with the Mut mutation is estimated at approximately 63% of the total MMA population. The number of MMA patients with the genetic deficiency targeted by LB-001 is estimated to be 3,400 to 5,100 patients in key global markets, of which 1,000 to 1,500 patients are in the United States.

Over time, patients with MMA typically develop end-stage renal disease requiring kidney transplantation in adolescence. Combined liver-kidney transplantation, or early liver transplantation, has emerged as an intervention aimed at improving metabolic control. However, the finite number of liver donors, significant risks associated with surgery, high procedural costs (in the United States, approximately $740,000 on average for liver transplantation and $1.2 million on average for combined liver and kidney transplantation (Milliman Research Report, 2014 U.S. organ and tissue transplant cost estimates)) and lifetime dependence on immunosuppressive drugs limit the widespread implementation of liver transplantation in patients with MMA.

Since MUT is a mitochondrial enzyme, deficiencies in MUT can be difficult or impossible to correct by enzyme replacement therapy in which functional enzyme is infused into the bloodstream. The most efficient way to get MUT enzyme inside the cell is to have it synthesized there. Several different approaches have been explored in animal models to accomplish this, including introducing mRNA to encode MUT directly into cells or introducing the gene for MUT into cells using a viral vector. While both of these approaches help to validate that the introduction of a functional MUT gene can ameliorate symptoms, they also each have a key limitation in that the therapeutic benefit is transient. In the case of mRNA therapy, weekly intravenous administration of the MUT mRNA was required to maintain therapeutic levels of MUT, but it is not clear how frequently this therapy would need to be administered in patients. In the case of MUT gene therapy, the levels of MUT decreased over time. Without a treatment that is durable, multiple doses would be required. However, the patient's development of neutralizing antibodies to the viral vector used to deliver the MUT gene therapy limits the ability to administer subsequent doses. In addition, administration of an AAV vector bearing a strong exogenous promoter has been correlated with hepatocellular carcinoma following neonatal delivery.

Introduction of a functional copy of the MUT gene into the genome of MMA patients would represent a much better approach, potentially providing lifelong therapeutic benefit from a single administration.

MMA is an organic acidemia with high unmet medical need and lack of therapeutic treatments. Because GENERIDE™ is designed to deliver therapeutic durability, it may provide lifelong benefit to MMA patients by intervening early in their lives with a treatment that restores the function of aberrant genes before irreversible declines in function can occur. In some embodiments, therapeutic transgenes are delivered using a GENERIDE™ construct designed to integrate immediately behind the gene coding for albumin, the most highly expressed gene in the liver. Expression of the transgenes “piggybacks” on the expression of albumin, which may provide sufficient therapeutic levels of desirable proteins given the high level of albumin expression in the liver.

### MMA Mouse Models

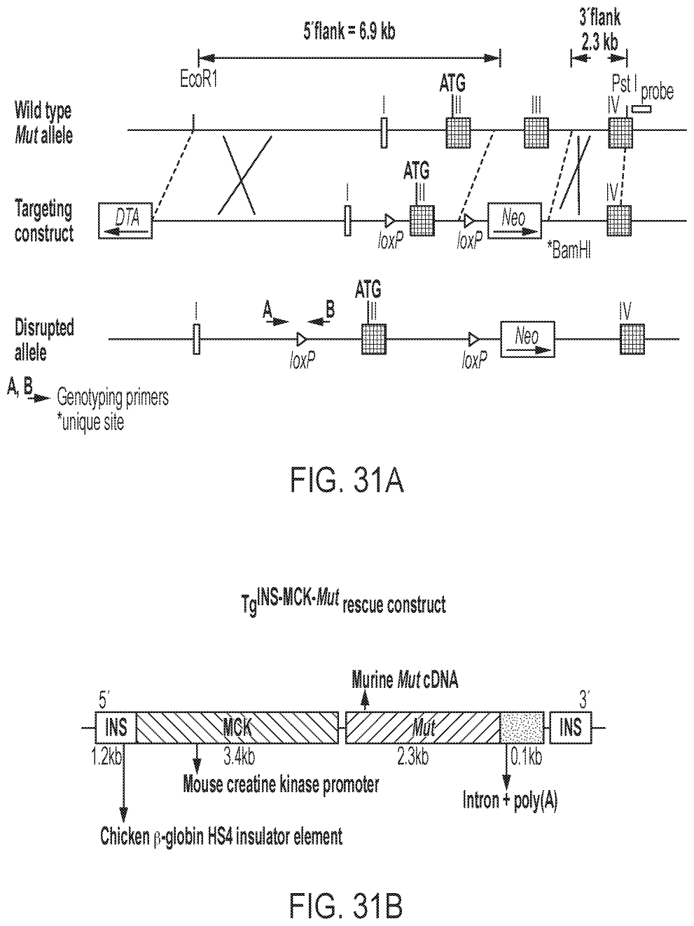

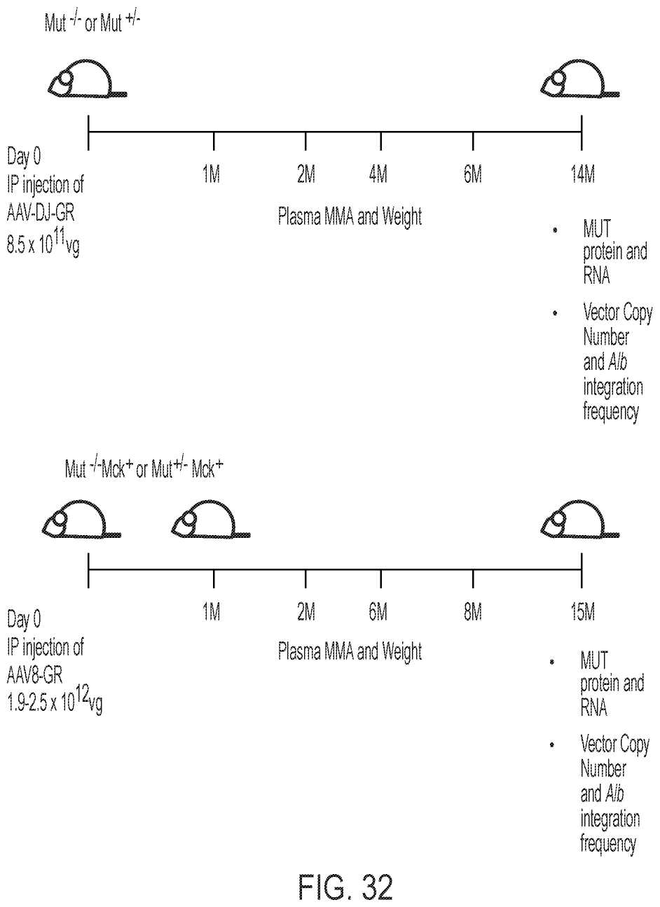

Murine models of MMA can be used to assay treatment with GENERIDE™ Exemplary murine models of MMA are depicted in FIG. 31A and FIG. 31B. Exemplary experimental methods for analysis of MMA mouse models after administration of GENERIDE™ constructs are illustrated in FIG. 32.

In one example of an MMA mouse model, the gene for Mut is rendered completely non-functional. This non-functional allele of Mut is referred to as Mut^{-/-}. Mice bearing this non-functional allele are believed to have a more severe deficiency than seen in the most severe cases of MMA in patients. Left untreated, these mice die within the first few days of life.

A modification of the Mut^{-/-} mouse is another mouse model of MMA called Mut^{-/-}; Tg^{INS-MCK-Mut}. As used herein, Mut^{-/-}; Tg^{INS-MCK-Mut} can be referred to as MCK-Mut or Mut^{-/-}; Mck-Mut or Mut^{-/-}MCK⁺. In this mouse model, there is a functional copy of the mouse Mut gene placed under the control of the creatine kinase promoter. This enables Mut expression in muscle cells, which in turn allows mice to survive longer while still exhibiting many of the phenotypic changes seen in MMA patients.

### EXEMPLIFICATION

### Example 1: Albumin as a Genomic Locus for Transgene Integration with GENERIDE™

The present example illustrates that the albumin locus can be a site of integration for transgene expression from the liver.

The albumin locus has several attractive features as a locus for transgene expression. A strong endogenous promoter drives high levels of albumin production and this strong promoter can be harnessed to maximize expression of a transgene to reach therapeutic levels without addition of a exogenous promoters. As illustrated in FIG. 4, albumin is highly expressed in the liver compared to other tissues. This liver-associated pattern of expression can be used for localizing expression of GENERIDE™ constructs predominantly to the liver. Additionally, as shown in FIG. 3, albumin is the highest-expressed gene in the liver and, relevantly, higher albumin expression relative to expression of disease-related genes in the liver can contribute to reaching therapeutic levels of transgene expression. For example, FIG. 5 illustrates that albumin expression levels are 100× higher than other select liver genes associated with monogenic diseases, including MMA.

### Example 2: LB-001 for the Treatment of Methylmalonic Acidemia (MMA)

The present example describes LB-001, a product candidate for the treatment of MMA. LB-001 contains a transgene coding for MUT, the most common gene deficiency in patients with MMA (FIG. 6). LB-001 is designed to target liver cells and insert the MUT transgene into the albumin locus.

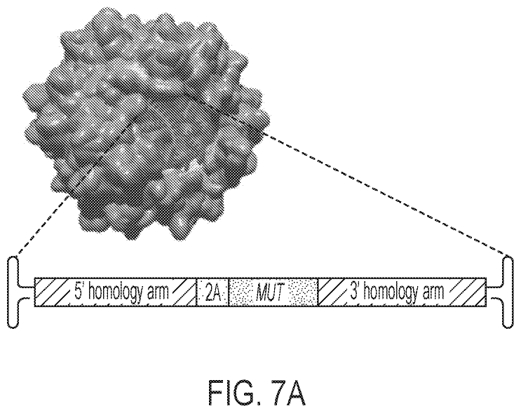

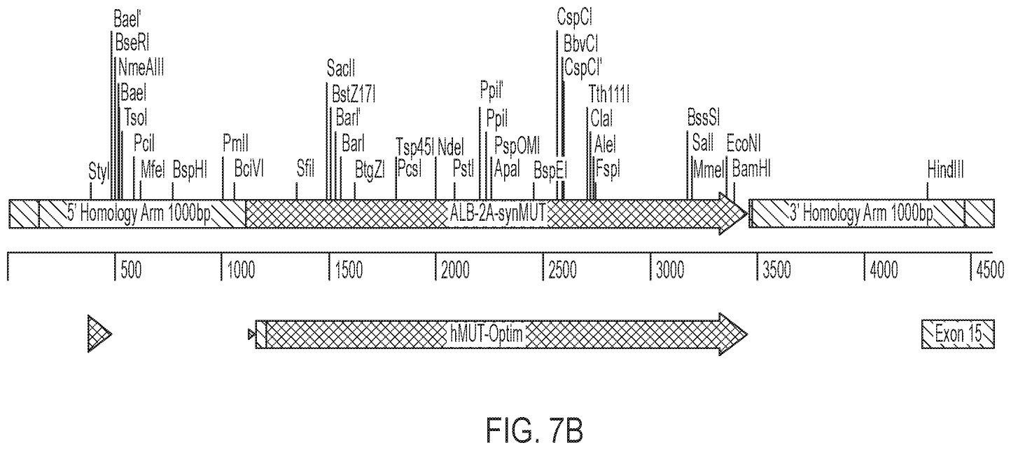

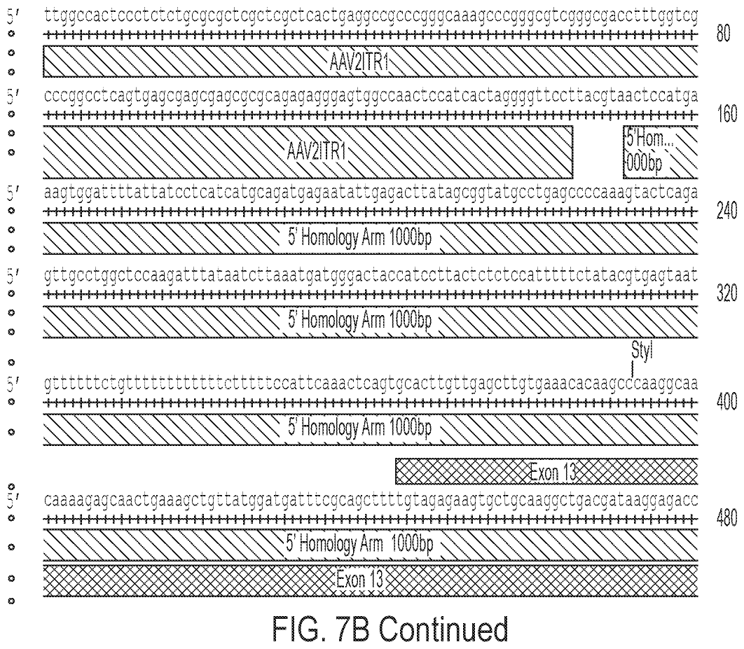

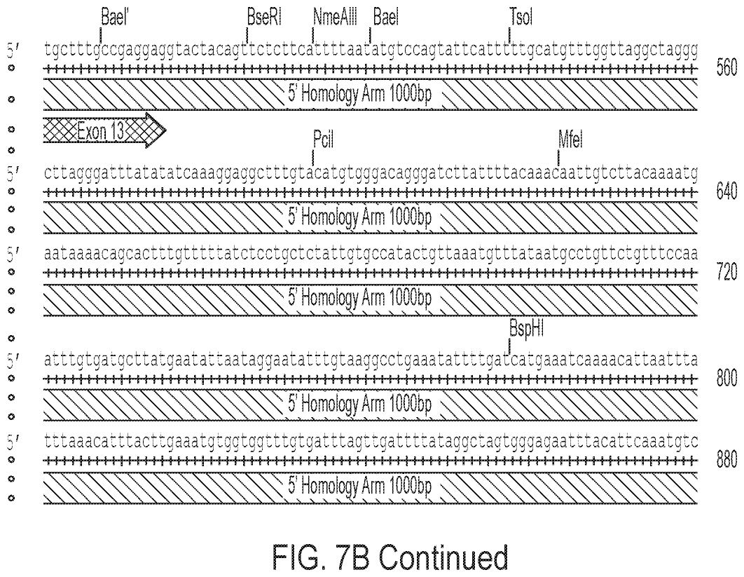

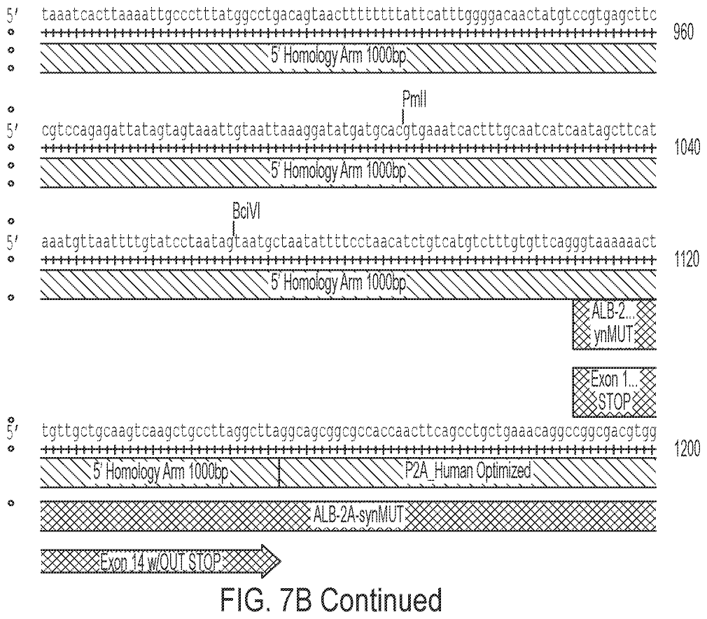

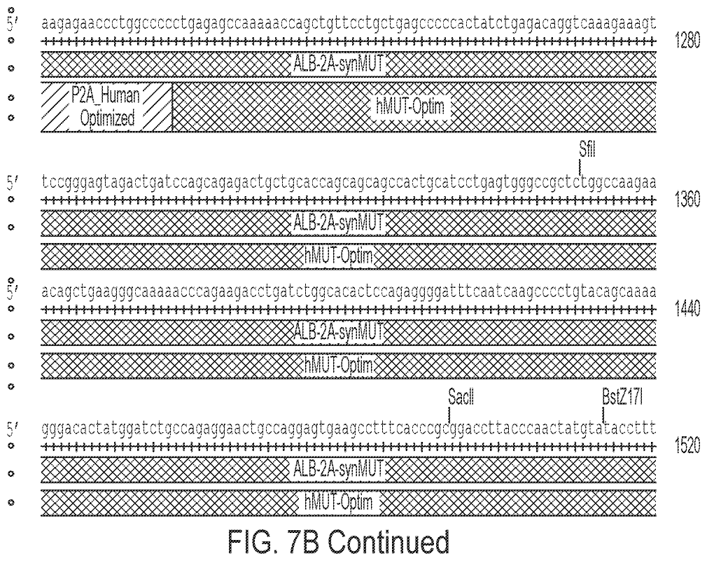

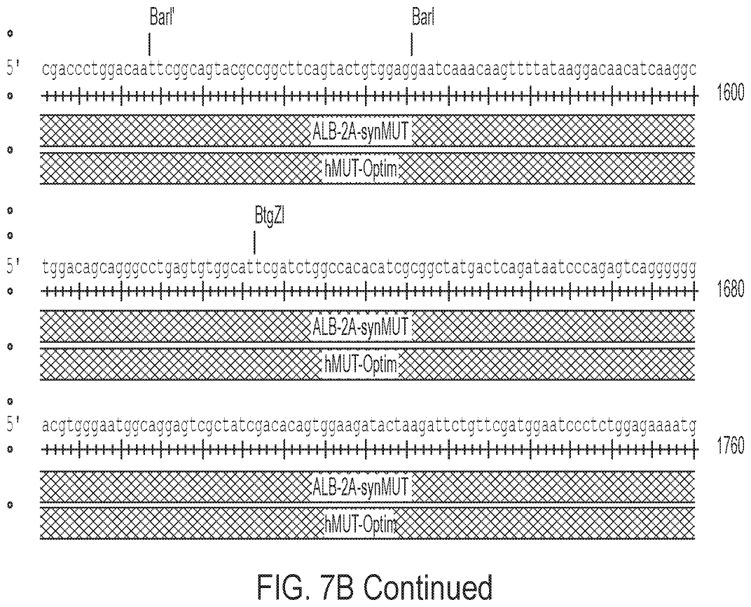

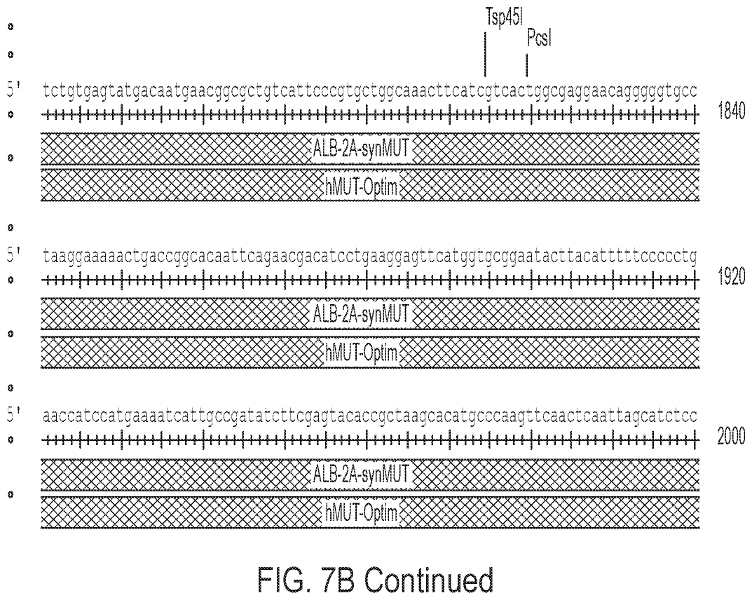

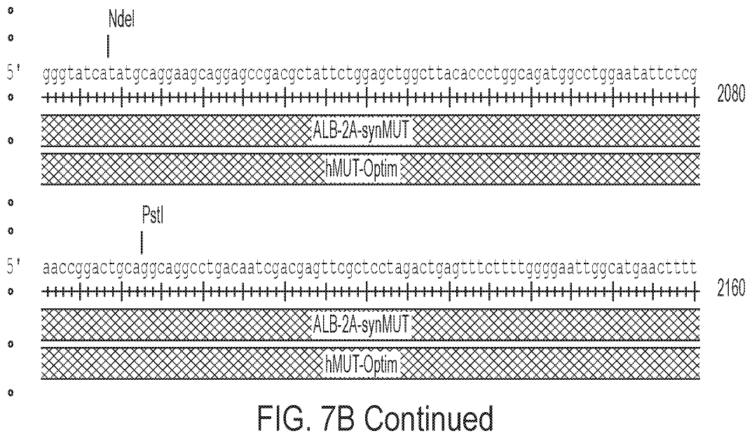

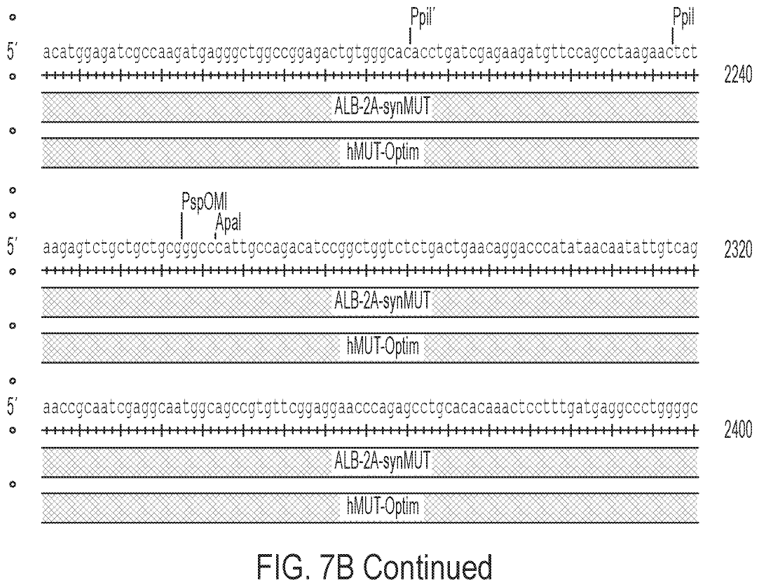

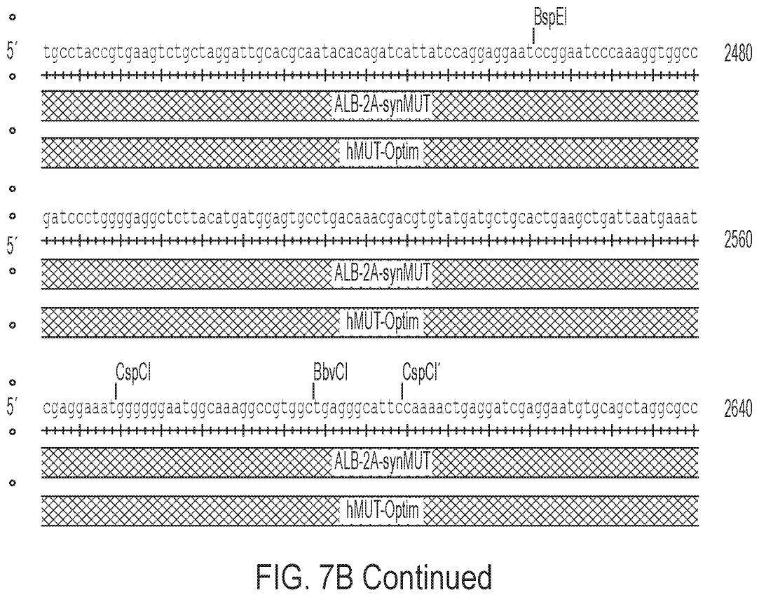

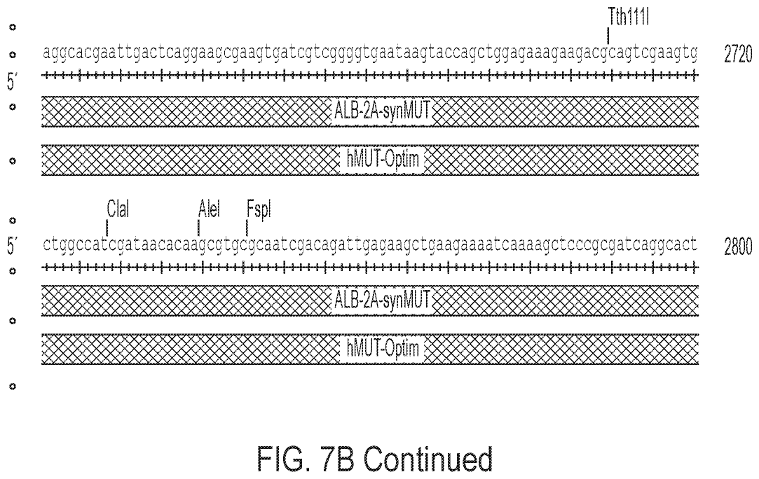

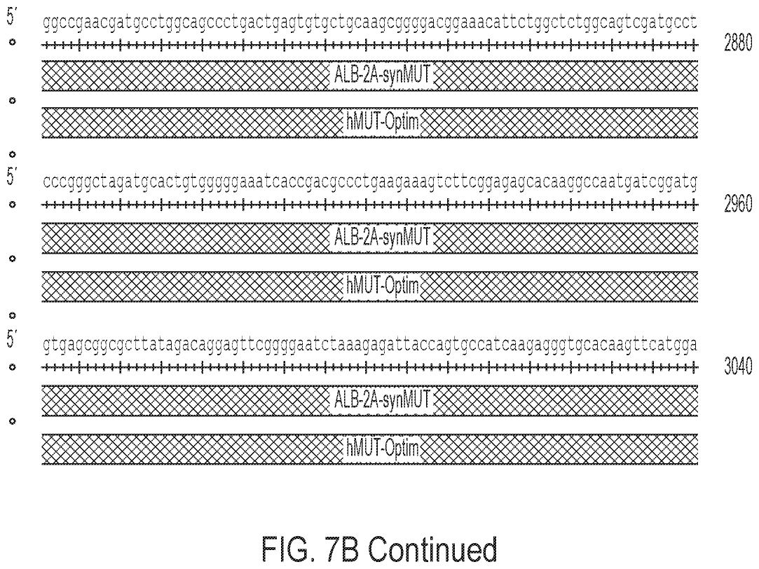

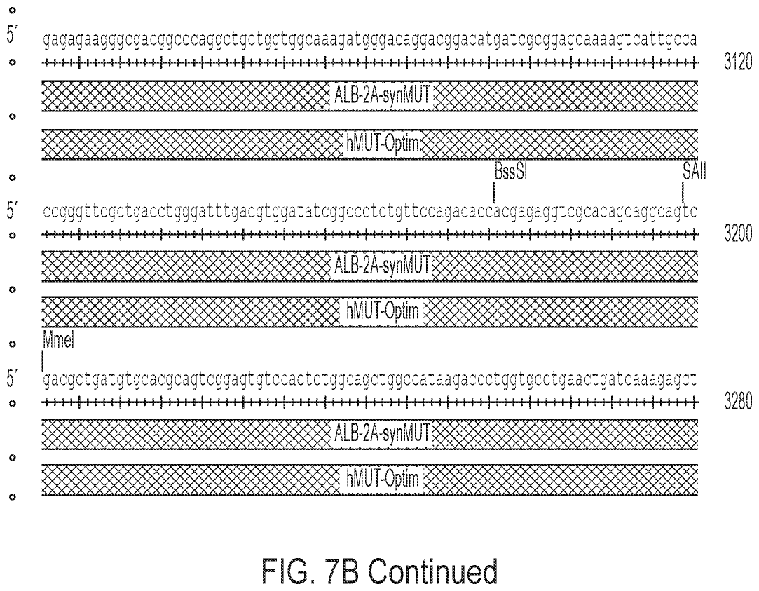

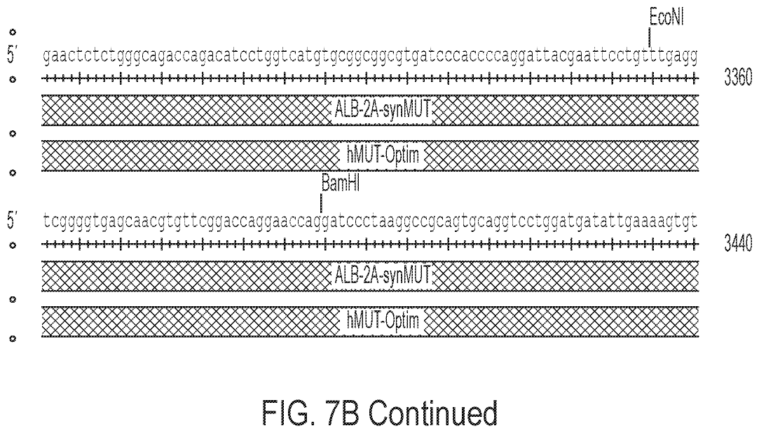

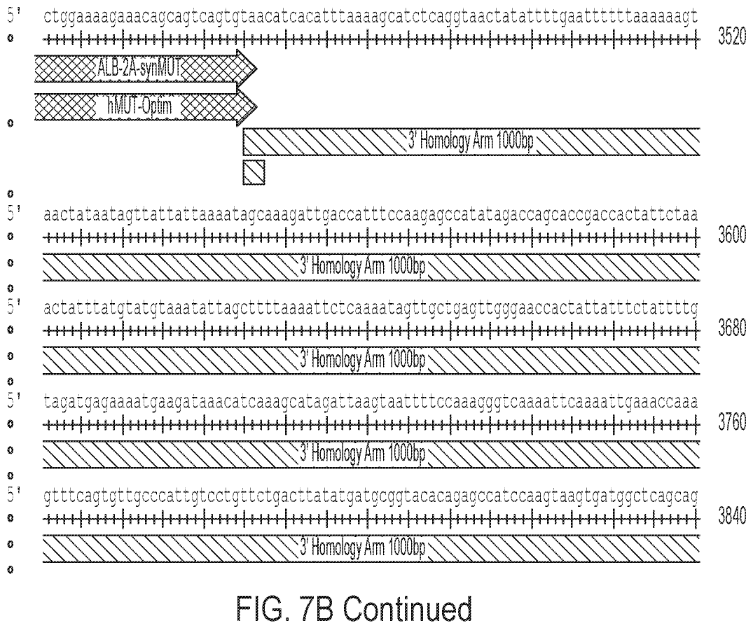

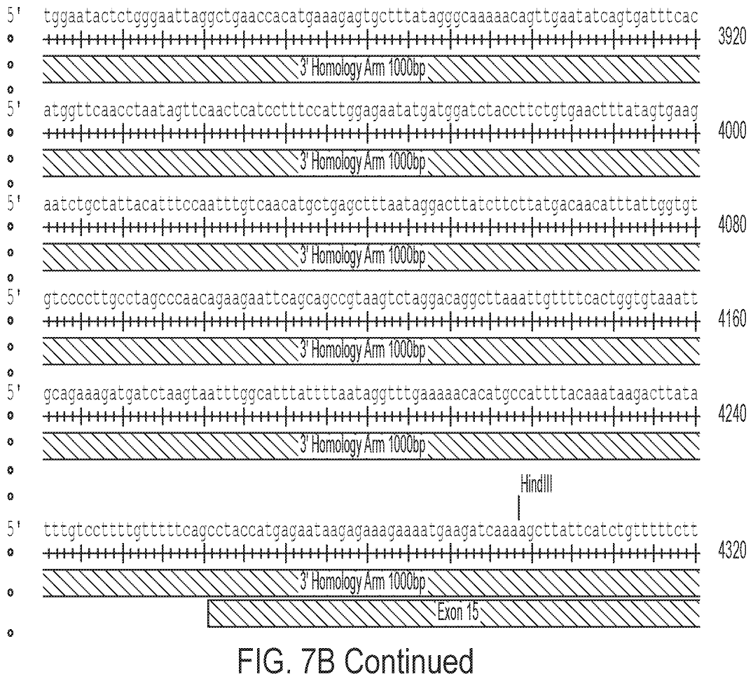

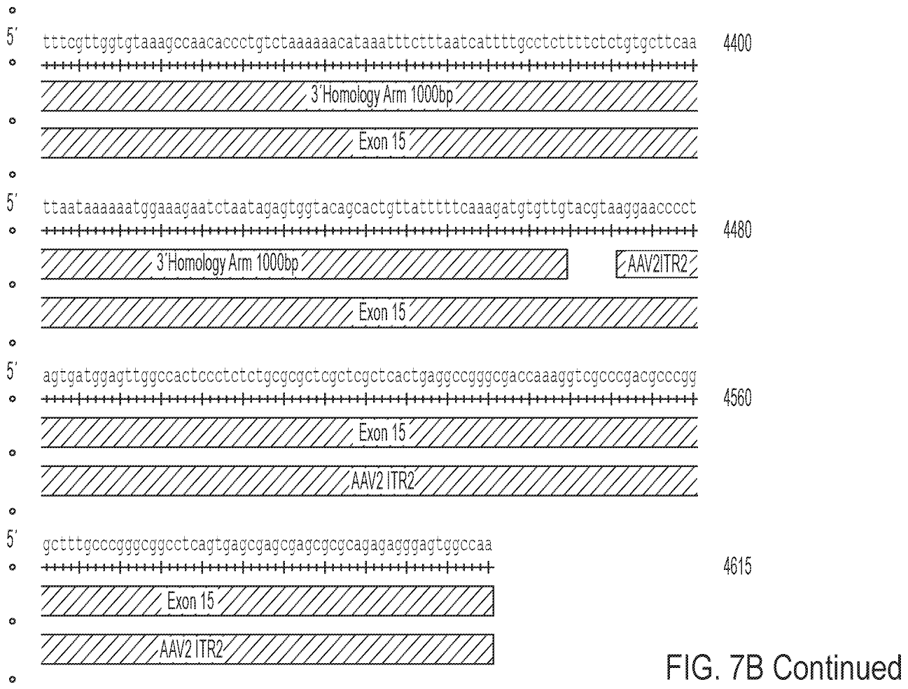

LB-001 consists of a DNA construct including a gene encoding the human MUT enzyme encapsulated in an AAV capsid (FIG. 7A). The MUT enzyme coding sequence is coupled to the 2A peptide sequence and surrounded by homology arms that drive the integration of the MUT gene and the 2A peptide sequence into the chromosomal locus for the albumin gene. Based on the way the construct integrates into the albumin locus, the MUT gene is expressed resulting in synthesis of MUT enzyme as a separate protein from albumin. LK03, an AAV capsid optimized to target human liver cells is used in LB-001.

An exemplary nucleic acid that can be used with the AAV-LK03 capsid to express a human Mut sequence is depicted in FIG. 7B. The nucleic acid comprises ITRs from AAV2, 1000 bases long 5′ and 3′ homology arms corresponding to an albumin sequence, and a synthetic human Mut sequence, preceded by a 2A-peptide to facilitate ribosomal skipping. A clinical indication for this construct includes treatment of severe methylmalonic acidemia (MMA) in combination with dietary management. Delivering a functioning copy of the methylmalonyl-CoA mutase (Mut) gene to the hepatocytes of MMA patients, using the GENERIDE™™ technology, is intended to clear and block the accumulation of toxic metabolites. Research grade LB-001 has been generated with triple transfection into HEK cells. Manufacture of clinical material can be done by known methods in the art, including using baculovirus expression vector system (BEVS) platforms.

### Example 3: Murine Dose Finding Analysis

The present example demonstrates an exemplary dose finding study design of an LB-001 surrogate in a Mut-MCK mouse model. Results from such an analysis can be applied to determine an efficacious dose of LB-001 surrogate on MUT knock-out mice when administered IV. Additionally, results from this analysis can provide a non-GLP toxicology evaluation and influence larger animal studies and clinical trials. For this example, the indication being evaluated is methylmalonic acidemia (MMA). Similar study designs can be incorporated for other indications.

In this study, the LB-001 surrogate comprises 1000 bp 5′ and 3′ homology arms. The vector (Vt-20 Batch 4 (CMRI)) is administered at the following three doses: 6e12 (Low), 6e13 (Mid), 6e14 (High) vg/kg. The mouse strain is Mut-MCK. Expected litter size of the animals is 6-8 pups. For each treatment group, it is estimated that 5-6 litters would be needed. Table 1 summarizes treatment groups in the study.

TABLE 1
Summary of treatment groups for dose finding analysis.

| Group | n | Treatment | Takedown | Readout |
|---|---|---|---|---|
| Blinded | 10 | Vehicle, IV injection, | 90 days | Survival, BW, MMA |
| | | p1 neonates | | plasma level |
| Blinded | 10 | LB-001 surrogate, | 90 days | Survival, BW, MMA |
| | | IV injection, p1 | | plasma level, liver |
| | | neonates, High dose | | integration |
| Blinded | 10 | LB-001 surrogate, | 90 days | Survival, BW, MMA |
| | | IV injection, p1 | | plasma level, liver |
| | | neonates, Mid dose | | integration |
| Blinded | 10 | LB-001 surrogate, | 90 days | Survival, BW, MMA |
| | | IV injection, p1 | | plasma level, liver |
| | | neonates, Low dose | | integration |

Sample collection for the study includes the following: (1) serum; (2) plasma (EDTA tubes); (3) liver (fresh frozen (dry ice), stored at −80 C)); and liver, kidney, heart, lung, brain, and skeletal muscle (10% formalin fixed overnight and stored at room temperature in 70% ethanol). Table 2 summarizes sample collection for the study.

TABLE 2
Summary of sampling for dose finding analysis.

| | | Mut −/− (Tg+) | | Mut +/− (Tg+ or Tg−) | |
|---|---|---|---|---|---|
| | | | Month 3 | | Month 3 |
| Genotype | | Months | (5 terminal, | Months | (5 terminal, |
| Sampling time | | 1, 2 | 5 survival) | 1, 2 | 5 survival) |
| Plasma MMA (50 μL) | | 10 | 10 | 5 | 5 |
| Plasma Alb-2A (10 μL) | | — | 5 | — | 5 |
| Serum ADA | | — | — | 5 | 5 |
| Serum chemistry (salts, | | — | — | 5 | 5 |
| liver/kidney panels) | | | | | |
| Liver, | Half fresh | — | 5 | — | 5 |
| weighing | frozen | | | | |
| whole | Half fixed | — | 5 | 3 | 5 |

| | | | | |
|---|---|---|---|---|
| Kidney, heart, brain, | — | — | 3 | 5 |
| skeletal muscle, fixed | | | | |

Readouts for the study includes the following: (1) survival; (2) body weight, measured once per week on a weekly basis; (3) MMA plasma level starting at D30, D60 and D90; and (4) integration in liver tissue at the end of the study (D90).

### Example 4: Efficacy of MUT Transgene Delivery in Mouse Models

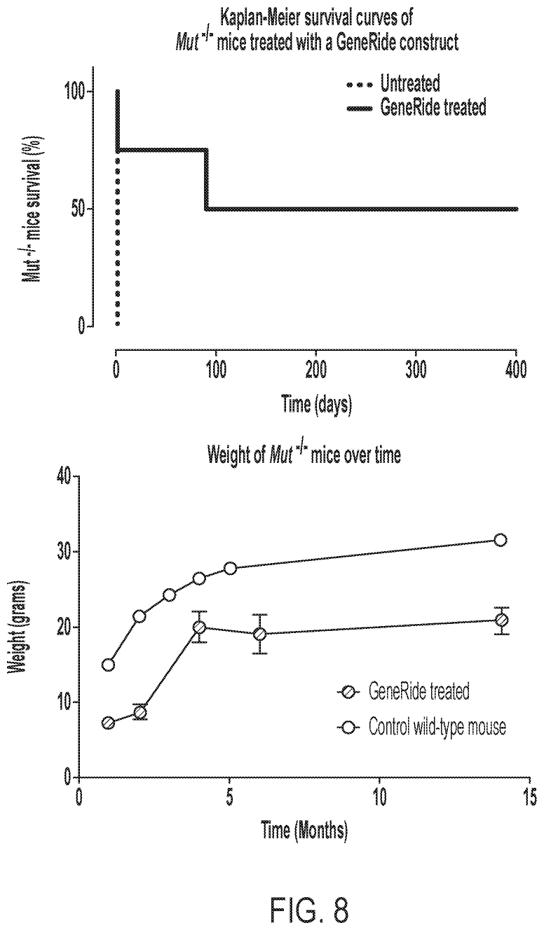

The present example provides preclinical data for LB-001 that was generated in two mouse models of MMA. In the first model, the gene for Mut had been rendered completely non-functional. This non-functional form of Mut is referred to as Mut−/−. Mice bearing this non-functional gene are believed to have a more severe deficiency than seen in the most severe cases of MMA in patients. Left untreated, these mice die within the first few days of life. A single intraperitoneal injection of a murine GENERIDE™ construct of LB-001 into four neonatal mice resulted in increased survival for three out of four mice, with two mice living for more than one year, as shown in the top panel of FIG. 8. In addition, these mice gained weight, when feeding freely, as shown in the bottom panel of FIG. 8.

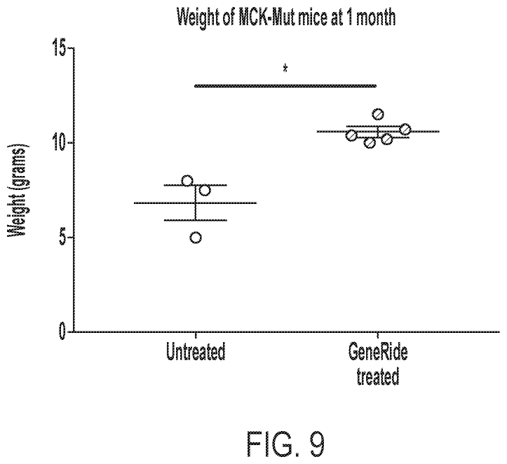

The second mouse model of MMA, called MCK-Mut, is a modification of the Mut−/− mouse in which a functional copy of the mouse Mut gene is placed under the control of the creatine kinase promoter. This allows Mut expression in muscle cells, which in turn allows mice to survive longer while still exhibiting many of the phenotypic changes seen in MMA patients. Five neonatal MCK-Mut mice received single injections of a murine GENERIDE™ construct of LB-001. Expression of Mut was observed in these mice. At one month of age, these mice had significant improvements in weight gain compared to untreated MCK-Mut mice, as shown in FIG. 9. These results were statistically significant. P-value is a standard measure of statistical significance, with p-values less than 0.05, representing less than a one-in-twenty chance that the results were obtained by chance, usually being deemed statistically significant.

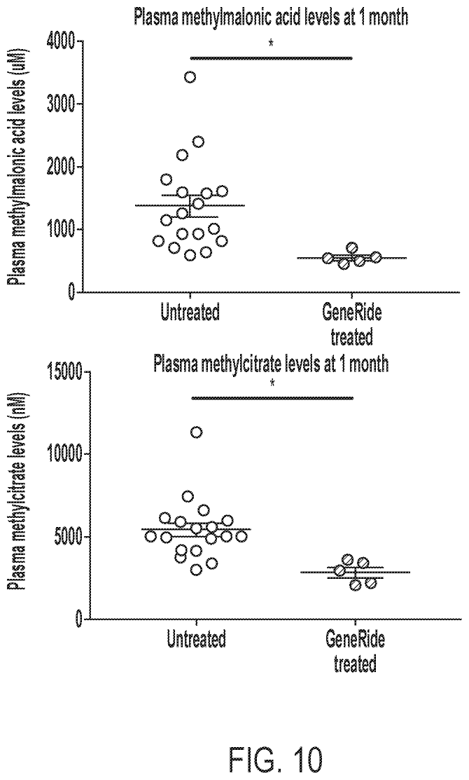

GENERIDE™-treated MCK-Mut mice also had significant reductions in plasma levels of methylcitrate and methylmalonic acid, disease-relevant toxic metabolites and diagnostic biomarkers that accumulate in patients with MMA, as shown in FIG. 10.

Surprisingly despite the relatively low rates of chromosomal integration achieved by AAV-directed HR gene editing, such methods result in therapeutic expression levels of functional Mut enzyme. Without wishing to be bound by any theory, it is hypothesized that this success is due to certain features of the LB-001 construct.

First, the AAV capsid utilized, LK03, has been optimized to target human liver cells. Second, genomic insertion is targeted into the locus for the albumin gene. Albumin is the most highly expressed protein in the liver and normal expression of most other proteins is only a fraction of that of albumin. Even a modest integration rate may, therefore, express therapeutic levels of protein. Transcriptionally active genes, of which albumin is one, are more susceptible to transgene integration using HR.

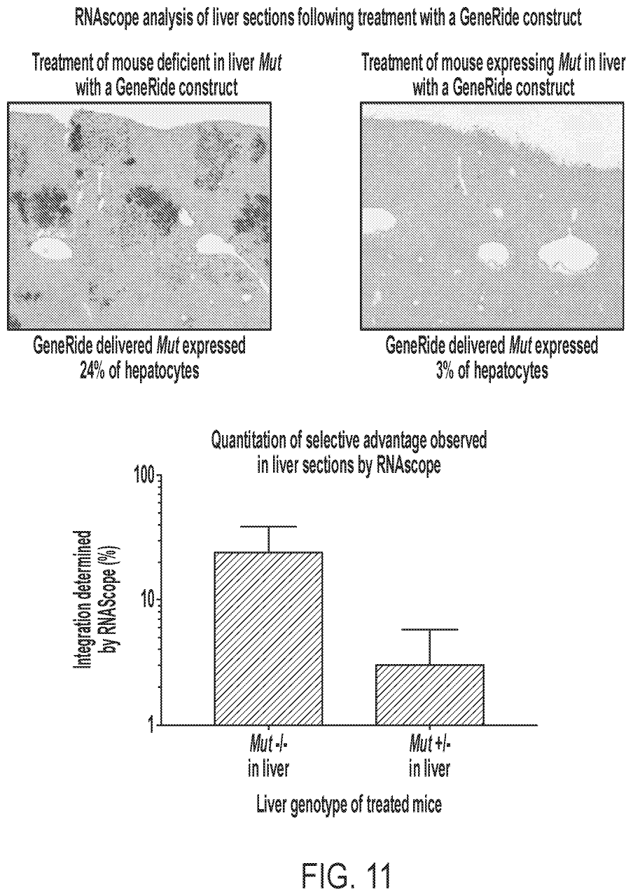

Third, the presence of a functional Mut enzyme itself has been observed to provide a selective advantage to hepatocytes over those lacking Mut. Over time, this selective advantage leads to an increased proportion of liver cells that contain the functional copy of Mut. This can be observed in mice in which a murine GENERIDE™ construct was introduced into mice with and without a functioning copy of Mut in the liver. The initial GENERIDE™ integration frequencies in both sets of mice were less than 4%. Over time, the number of modified cells remained the same in mice that naturally express Mut in the liver (Mut+/− in liver). However, after more than one year, in the mice genetically deficient in liver Mut (Mut−/− in liver), the percent of cells expressing Mut increased to 24% as shown in FIG. 11. Without wishing to be bound by any theory, this selective advantage may be attributable to improvements in mitochondrial function as a result of Mut expression and restoration of the deficient amino acid metabolic pathway.

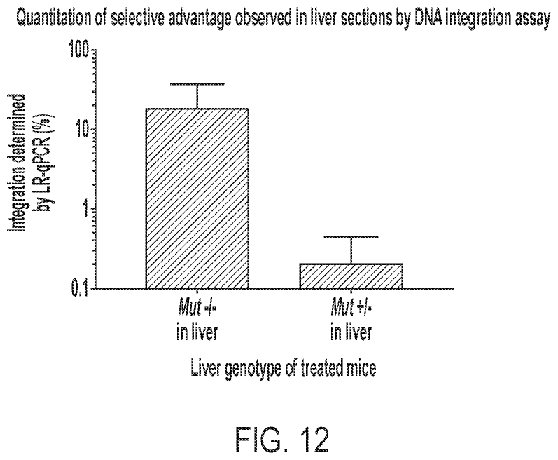

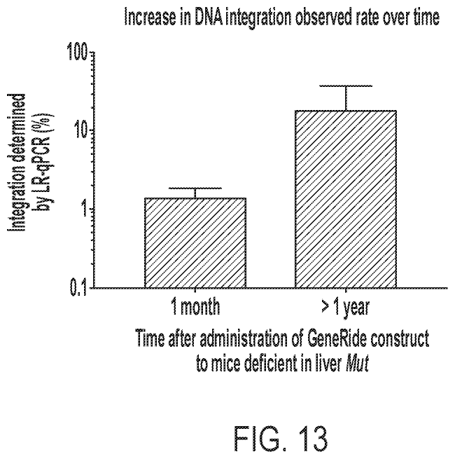

Additional supporting evidence for selective advantage in these mice includes (i) quantification of cells with the Mut gene integrated at the albumin locus by an orthogonal long-range quantitative polymerase chain reaction, or LR-qPCR, as shown in FIG. 12 and (ii) detection of an increased rate of integration at the albumin locus by LR-qPCR at more than one-year compared to one month post dose, as shown in FIG. 13.

In contrast to conventional AAV gene therapy approaches, in which the percentage of cells containing the therapy decreases over time as cells replicate and lose the virally encoded transgene, in the MMA mouse study, the percentage of cells containing a Mut GENERIDE™ construct increased over time. These results support the possibility that a single administration may provide lifelong benefits.

### Example 5: Efficacy of MUT Transgene Delivery in Mouse Models

The present example confirms the findings presented in Example 4. As in Example 4, the present example uses a promoterless AAV vector that utilizes homologous recombination to achieve site-specific gene addition of human MUT into the mouse albumin (Alb) locus. This vector (AAV-Alb-2A-MUT) contains arms of homology flanking a 2A-peptide coding sequence proximal to the MUT gene, and generates MUT expression from the endogenous Alb promoter after integration. Previous data has indicated that AAV-Alb-2A-MUT, delivered at a dose of 8.6E11-2.5E12 vg/pup at birth, reduced disease related metabolites, and increased growth and survival in murine models of MMA (Chandler, R. J. et al., Rescue of Mice with Methylmalonic Acidemia from Immediate Neonatal Lethality Using an Albumin Targeted, Promoterless Adeno-Associated Viral Integrating Vector, Molecular Therapy, Abstract 26, 25(5S1): page 13 (May 2017)). The present example, like Example 4, discloses the finding that MUT transgene delivery with the constructs and methods disclosed herein confers longer-term efficacy in MMA mouse models.

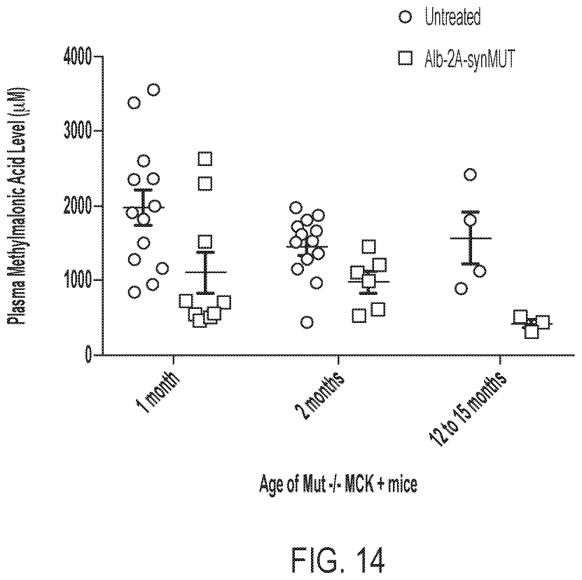

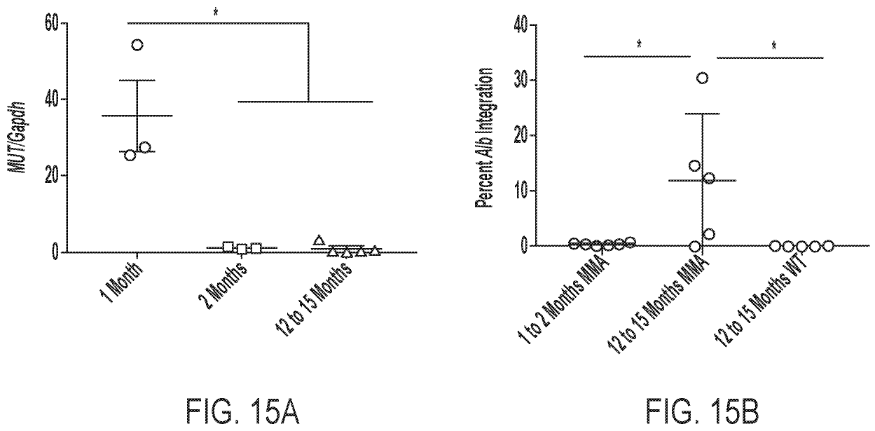

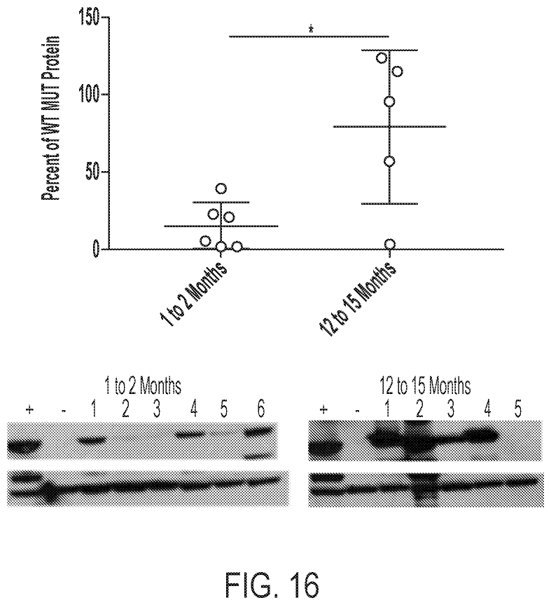

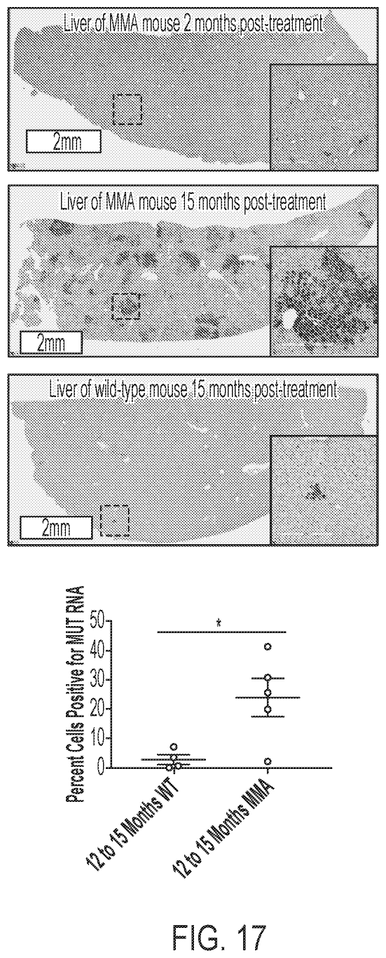

As presented in Example 4, the present example confirms that treatment of a hypomorphic MMA murine model with GENERIDE™ results in reduction in plasma levels of methylmalonic acid (FIG. 14). Also as presented in Example 4, the present example confirms that MUT transgene integration confers hepatocellular growth advantage in mice with MMA. For instance, hepatice MUT protein expression, percentage of MUT mRNA cells, and the number of Alb-integrations were observed to increase over time in treated MMA mice (FIGS. 15-17). The low levels of transgene integrations and low numbers of MUT mRNA positive cells observed in wild-type mice 13-15 months post-treatment and MMA mice 2 months post-treatment (FIGS. 15 and 17), are characteristic of correction by in vivo homologous recombination.

Additionally, as in Example 4, the present example shows that RNAscope of AAV-Alb-2A-MUT treated MMA mice revealed robust MUT expression, and MUT positive hepatocytes appeared as distinct and widely dispersed clusters, consistent with a pattern of clonal expansion. RNAscope studies also show that the MUT expression was present in approximately 5-40% of the hepatocytes in treated MMA mice versus 1% in wild-type controls (FIG. 17). The findings of Example 4 and the present example indicate that a selective advantage for corrected hepatocytes can be achieved in murine models of MMA after treatment using MUT GENERIDE™. This observation has clinical relevance for treating MMA patients.

### Example 6: Efficacy of MUT Transgene Delivery in Mouse Models

The present example confirms the findings presented in Example 4 for treatment of MMA mouse models with murine LB001.

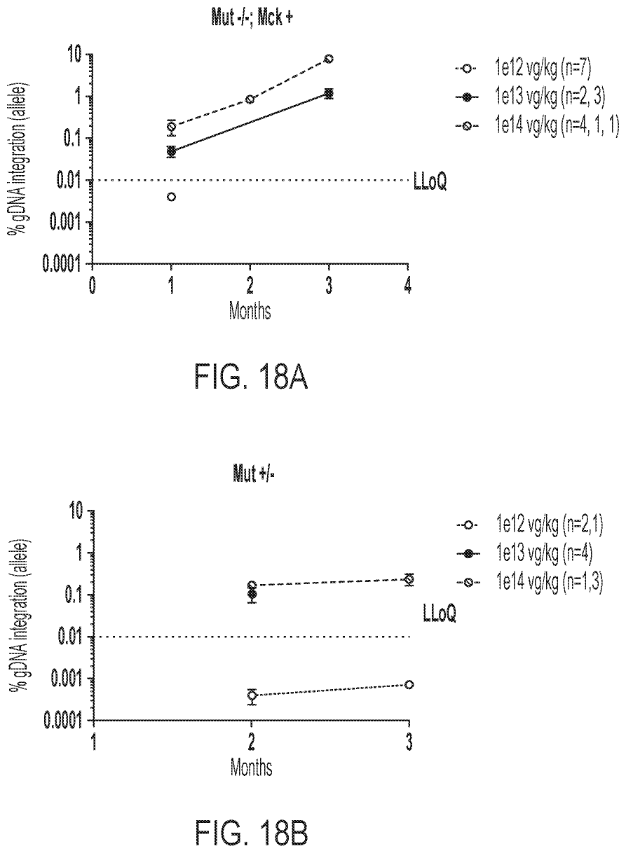

As in Example 4, the present example discloses increase in DNA integration over time for MMA mouse models deficient in liver MUT (FIG. 18). This increase was observed for different doses of the transgene construct. Without wishing to be bound by any theory, such an increase in transgene integration using the construct and methods disclosed herein, such an observed selective advantage may be harnessed for purposes of achieving therapeutic levels of transgene expression at a safe dose of construct administration to patients. For example, beginning with a relatively low dose of construct, a patient suffering from MMA could eventually reach sufficient levels of MUT transgene to reduce the severity or treat the disease. Observation of increased transgene integration over time in patients could be used to confirm monitor treatment.

### Example 7: Investigating In Vivo Activities of hLB001 in a Humanized Mouse Model

This example provides an exemplary analysis to evaluate the efficacy of site-specific integration of a MUT transgene into the human ALB locus using recombinant AAV (hLB001) (LK-03-GENERIDE™ MUT) and the humanized FRG KO/NOD murine model.

The vector for this analysis is hLB001 administered to FRG mice with humanized liver at 2 dosing levels (1e13 and 1e14 vg/kg). Endpoints for this analysis include the following: (1) Percentage of genomic integration and (2) Expression of ALB-2A-MUT fused mRNA. The timepoint to be analyzed includes 21 days post infection.

### Materials, Methods, and Sampling

Materials
a. 3, female humanized Fah^{-/-}/Rag2^{-/-}/Il2rg^{-/-} NOD mice (Hu-FRGN) with ≥80% human hepatocyte replacement with donor HHM19027/YTW
b. 12, female humanized Fah^{-/-}/Rag2^{-/-}/Il2rg^{-/-} NOD mice (Hu-FRGN) with ≥80% human hepatocyte replacement with donor HHF13022/RMG
c. Yecuris human albumin ELISA
d. Sterile 3/10 cc syringe with a 29 g needle
e. Sterile 1 cc syringe with a 29 g needle
f. Sodium Citrate coated tubes, 0.8 mL
g. PBS, vehicle
h. Preliminary Phase: rAAV, titer: 6.43e13 vg/mL
i. Phase 1: rAAV titer: 9.29e13 vg/mL
j. 1.5 mL tubes, sterile
k. Mouse Anesthetic cocktail (7.5 mg/mL ketamine, 1.5 mg/mL Xylazine and 0.25 mg/mL Acepromazine)
l. TissueTek cassettes
m. 10% Normal Buffered Formalin, prepared fresh
n. Ethanol, 70%
o. 5 mL polypropylene tube with screw cap
p. Liquid nitrogen

Methods

Preparation of Mice Prior to Dosing:

All mice to be used in the study will be removed from NTBC≥25 days and SMX/TMP≥3 days prior to initiation of the study. Humanization will be evaluated ≤7 days prior to start of study.

Preparation of Virus for Dosing:

Virus should be thawed and kept on ice during and after preparation. The PBS could be thawed at 37 C or room temperature. It is suggested to thaw the PBS≥30 minutes and the virus≥5 minutes prior to preparation.

Preliminary Study—Pilot:
a. Compound Formulation:
   i. To deliver a 1e14 vg/kg need a 2e13vg/mL stock of virus
   ii. Inside a Biosafety cabinet, level II, dilute the 6.43e13 vg/mL to 2e13 vg/mL. Assume an average body weight of 25 g

| # of Mice to | Virus | PBS, sterile | Total volume |
|---|---|---|---|
| dose | (6.43e13/vg/μl) | (μL) | (μL) |
| 3 | 155 | 345 | 500 |

b. Four (4) HuFRGN transplanted with HHM19027/YTW will be divided into two groups and dosed with the indicated compounds at the indicated dose outlined in the chart below

| | Number of | Dosing | Dose | |
|---|---|---|---|---|
| Group | mice | compound | (vg/Kg) | |
| 1 | 1 | Vehicle | 5 | mL/Kg |
| 2 | 3 | rAAV | 1e14 | vg/kg |

c. On Day 1 each group will receive the designated dose of each compound by intravenous delivery via the retro-orbital sinus vein using a sterile 3/10 cc needle with a 29 g needle:
   iii. Each mouse will be weighed and the body weight (BW) will be recorded.
   iv. The BW (g) of each mouse will be multiplied by the concentration of the stock solution in vg/g to determine the total vg of compound needed to achieve the desired dose.
   v. The total number of vg will be divided by the concentration of the stock solution in vg/μL to determine the volume of the stock solution to use for dosing.
   vi. The mice will be anesthetized using vaporized isoflurane prior to dosing.
   vii. The calculated dose of virus for each mouse will be drawn into a sterile 29G needle on a 3/10 cc syringe and delivered via the retro-orbital sinus vein
d. All animals will be monitored immediately after dosing to ensure recovery from anesthesia and there was no unintended harm done to the animal during dosing.
e. All mice will be monitored every day for general health. If a mouse is found moribund or deceased the mouse will be anesthetized and samples will be collected as described below in the “Terminal Harvest” section.

Terminal Harvest
a. On day 22 (three weeks post dosing) all mice will be weighed and anesthetized using Mouse cocktail according to the body weight.
b. As much whole blood as possible will be collected via cardiac puncture using a 1 cc syringe with a 27 g needle. The whole blood will be transferred into a Sodium Citrate coated tube, plasma will be isolated by centrifugation at 1500×g for 15 minutes at 4° C. The plasma will be dispensed into 1004, aliquots and stored at −80° C.
c. The peritoneum and thoracic cavity will be opened to expose the liver, the liver will be isolated and the weight of the liver recorded. The liver will be dissected into the individual lobes, each lobe will be further dissected into two equal parts.
d. For histology one pieces from each lobe will be placed in a TissueTek cassette and fixed in freshly prepared 10% normal buffered formalin for 16-32 hrs at room temperature, then transfer to 70% Ethanol and stored at room temperature.
   NOTE: Do not fix at 4° C. Do not fix for <16 hrs or >32 hrs. Delayed fixation can degrade RNA and produce lower signal or no signal. Shorter time or lower temperature will result in under-fixation.
e. For bioanalysis the second piece from each lobe will be transferred to a 5 mL polypropylene tube and flash frozen in liquid nitrogen and stored at −80° C.

Study—Phase 1
a. Compound Formulation:
   i. To deliver a 1e14vg/kg need a 2e13vg/mL stock of virus. To deliver 1e13vg/kg need a 2e12vg/mL
   ii. Inside a Biosafety cabinet, level II, dilute the 9.29E+13 vg/mL to 2e13 vg/mL and 2e12 vg/mL stock. Assume an average body weight of 25 g

| | | Virus | PBS, | Total |
|---|---|---|---|---|
| # of Mice to | Dose | (9.25E + 13 | sterile | volume |
| dose | (vg/mL) | vg/mL) | (μL) | (μL) |
| 5 | 2e13 | 181 | 669 | 850 |
| 5 | 2e12 | 18 | 832 | 850 |

b. Twelve (12) HuFRGN transplanted with HHF13022/RMG will be divided into three groups and dosed with the indicated compounds at the indicated dose outlined in the chart below.

| | Number of | Dosing | Dose | |
|---|---|---|---|---|
| Group | mice | compound | (vg/Kg) | |
| 1 | 2 | Vehicle | 5 | mL/Kg |
| 2 | 5 | rAAV | 1e14 | vg/kg |
| 3 | 5 | rAAV | 1e13 | vg/kg |

c. On Day 1 each group will receive the designated dose of each compound by intravenous delivery via the retro-orbital sinus vein using a sterile 3/10 cc needle with a 29 g needle:
   iii. Each mouse will be weighed and the body weight (BW) will be recorded.
   iv. The BW (g) of each mouse will be multiplied by the concentration of the stock solution in vg/g to determine the total vg of compound needed to achieve the desired dose.
   v. The total number of vg will be divided by the concentration of the stock solution in vg/μL to determine the volume of the stock solution to use for dosing.
   vi. The mice will be anesthetized using vaporized isoflurane prior to dosing.
   vii. The calculated dose of virus for each mouse will be drawn into a sterile 29G needle on a 3/10 cc syringe and delivered via the retro-orbital sinus vein
d. All animals will be monitored immediately after dosing to ensure recovery from anesthesia and there was no unintended harm done to the animal during dosing.
e. All mice will be monitored every day for general health. If a mouse is found moribund or deceased the mouse will be anesthetized and samples will collected as described below in the “Terminal Harvest” section

Terminal Harvest
a. On day 22 (three weeks post dosing) all mice will be anesthetized using Mouse cocktail.
b. As much whole blood as possible will be collected via cardiac puncture using a 1 cc syringe with a 27 g needle. The whole blood will be transferred into a Sodium Citrate coated tube, plasma will be isolated by centrifugation at 1500×g for 15 minutes at 4° C. The plasma will be dispensed into 1004, aliquots and stored at −80° C.
c. The peritoneum and thoracic cavity will be opened to expose the liver, the liver will be isolated and the weight of the liver recorded. The liver will be dissected into the individual lobes, each lobe will be further dissected into two equal parts.
d. For histology one pieces from each lobe will be placed in a TissueTek cassette and fixed in freshly prepared 10% normal buffered formalin for 16-32 hrs at room temperature, then transfer to 70% Ethanol and stored at room temperature.
   NOTE: Do not fix at 4° C. Do not fix for <16 hrs or >32 hrs. Delayed fixation can degrade RNA and produce lower signal or no signal. Shorter time or lower temperature will result in under-fixation.
e. For bioanalysis the second piece from each lobe will be transferred to a 5 mL polypropylene tube and flash frozen in liquid nitrogen and stored at −80° C.

### Example 8: GENERIDE™ on Primary Human Hepatocytes

Primary human hepatocytes were cultured using sandwich culture system. Cells were infected by GENERIDE™™ hLB001 for 48 hours before media change. 7 days post infection, cells were harvested, and RNA was extracted using Qiagen Allprep kit (Cat No./ID: 80204).

After RNA extraction, 1 μg of RNA was used for the reverse transcription by High-Capacity cDNA Reverse Transcription Kit (Thermofisher 4368814). cDNA was used as template for downstream PCR amplification by primers 235/267 (FIG. 19). PCR product was sequenced with primer 235.

Sequencing result shows the fused mRNA of ALB exon 12, exon 13, exon 14 before stop codon and 2a sequence which represents the correct expression of fused mRNA from precise integration mediated by GENERIDE™™ on primary human hepatocytes.

### Example 9: GENERIDE™ on Primary Human Hepatocytes

The present example confirms the results observed in Example 8, in that the GENERIDE™ vector LB001 can mediate efficient genome editing of MUT into the ALB locus in human primary hepatocytes.

### Methods

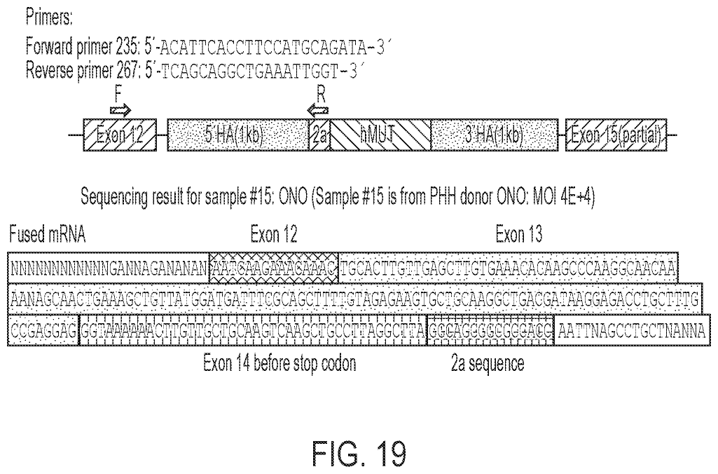

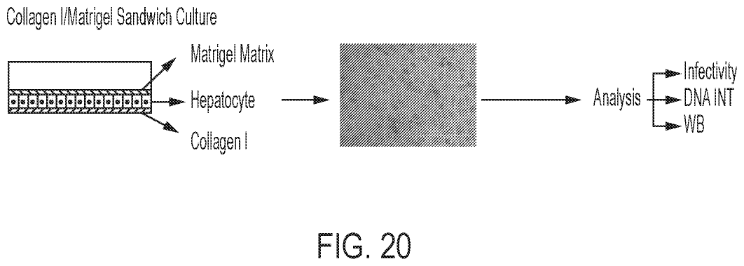

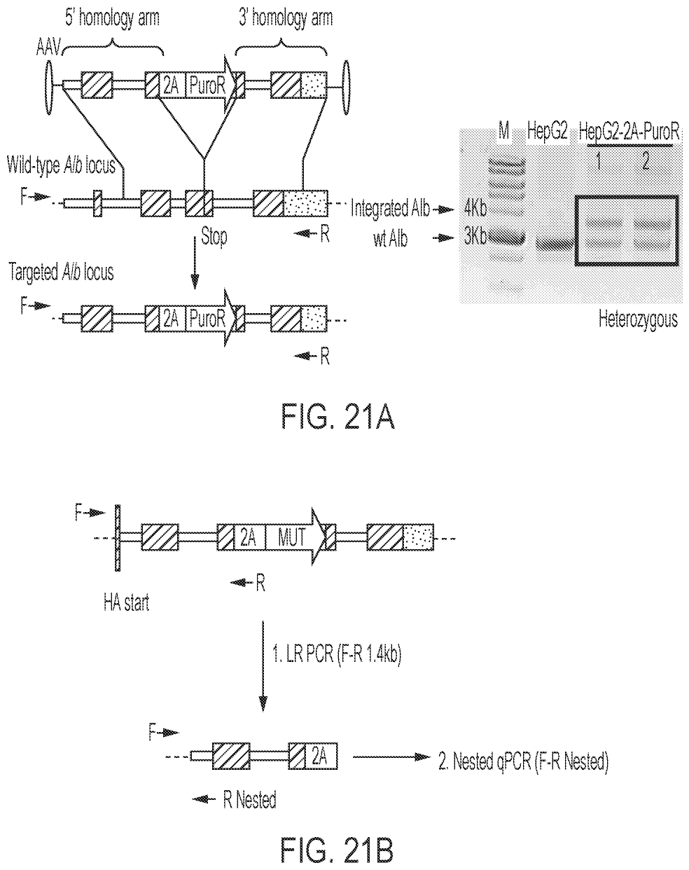

A primary human hepatocyte sandwich culture system was utilized to analyze infectivity, DNA integration, and protein levels (FIG. 20). Site-specific integration rate was analyzed using Long-range (LR) qPCR (FIG. 21). A stable HepG2-2A-PuroR cell line was used as positive control in DNA.

### Results

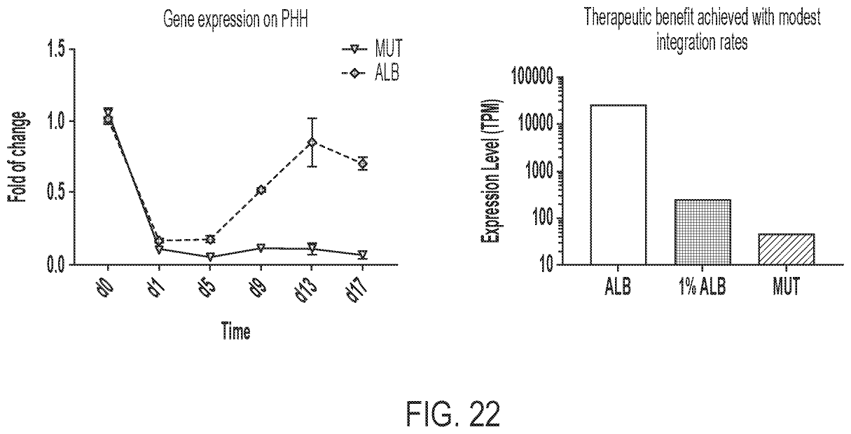

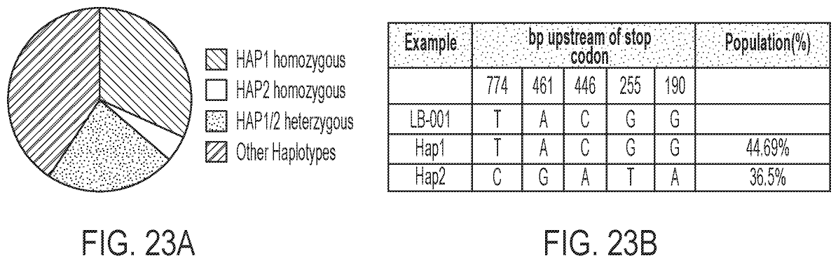

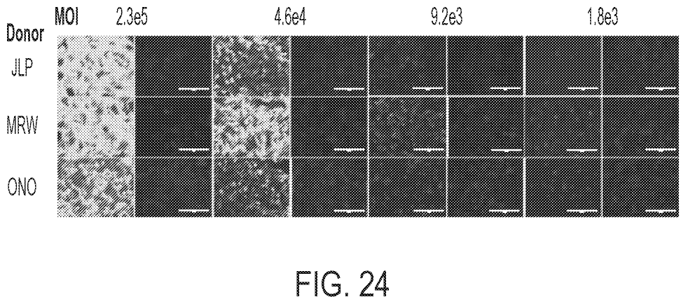

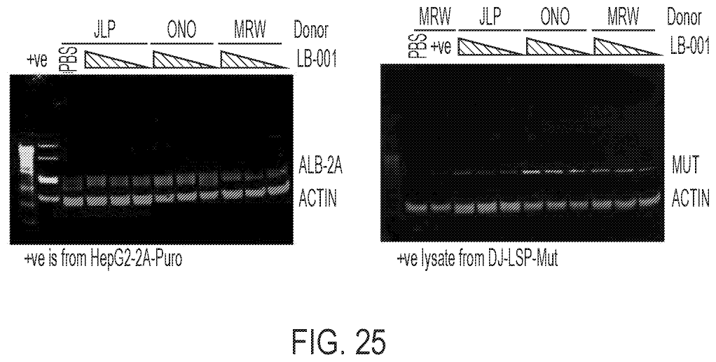

Relative expression of MUT and ALB were assessed (FIG. 22). For additional studies, three primary human hepatocyte donors with the same haplotype 1 were chosen to test GENERIDE™ LB-001 (FIGS. 23-25). These results confirm that GENERIDE™ LB-001 can integrate and express the MUT transgene in primary human hepatocytes.

### Example 10: MUT Transgenes for Applications in GENERIDE™ Technology to Treat MMA

The present example shows that different MUT transgenes can be used for applications in GENERIDE™ technology. For example, synthetic polynucleotides encoding a human methylmalonyl-CoA mutase (synMUT) may be used in GENERIDE™ applications. Examples of synMUT constructs are described in WO/2014/143884 and U.S. Pat. No. 9,944,918, both incorporated herein by reference. Exemplary optimized nucleotide sequences encoding human methylmalonyl-CoA mutase (synMUT1-4) are listed as SEQ ID NOs: 9, 12, 13, and 14, respectively.

### Example 11: Inborn Errors of Metabolism

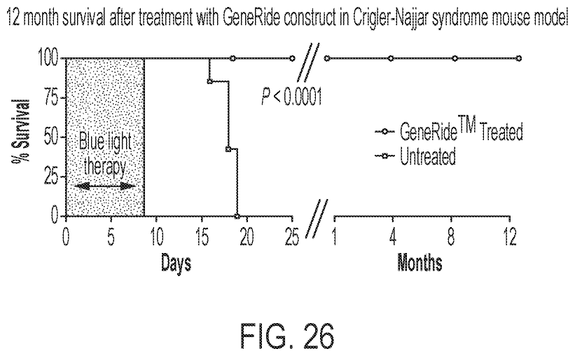

The liver is a key organ responsible for many metabolic and detoxifying processes. Dozens of monogenic disease, including MMA, arise from deficiencies in liver enzymes involved in metabolic pathways. Additional proof of concept data has been generated in animal models to address another rare inborn error of metabolism, Crigler-Najjar syndrome. Patients with Crigler-Najjar are unable to metabolize and remove bilirubin from circulation, resulting in lifelong risk of neurological damage and death. A similar GENERIDE™ construct, but with the gene for bilirubin uridine diphosphate glucuronosyl transferase, or UGT1A1, as the transgene, was used to correct the gene deficiency in an animal model of Crigler-Najjar syndrome. The introduction of UGT1A1 into the albumin locus in mouse liver cells resulted in normalization of bilirubin levels and long-term survival of mice deficient in UGT1A1 from less than twenty days to at least one year, as shown in FIG. 26. Additional indications that can be pursued in this category include phenylketonuria, ornithine transcarbamylase deficiency and glycogen storage disease type 1A.

### Example 12: Other Liver-Directed Therapies

The specificity of therapeutic product candidates for the liver is determined both by the AAV capsid used and by the location of integration into the host cell's DNA. LB-001 utilizes the AAV capsid, LK03, which was designed to be highly efficient for transduction of human liver. The transgenes for liver directed therapeutic product candidates were inserted into the albumin gene locus, which is only produced at a meaningful level in the liver, where it is the most highly expressed gene. The selection of albumin is considered to enhance liver specificity because the active transcription enhances the rate of homologous recombination and the tissue-specific expression of the albumin gene will drive production of a transgene in the liver.

### Example 13: Using Liver as In Vivo Protein Factory

This example illustrates that the modulatory design of GENERIDE™ can be applied for production of proteins that function outside of the liver.

The liver is a major secretory organ that produces many proteins found in circulation. This attribute can allow hepatocytes to deliver key therapeutic proteins to patients with genetic deficiencies. For example, this has been demonstrated in an animal model of hemophilia B using a murine GENERIDE™ construct of LB-101, encoding human coagulation factor IX to correct a clotting deficiency. In this model, expression of human coagulation factor IX and blood coagulation was restored to normal levels after a single treatment in neonatal and adult diseased mice.

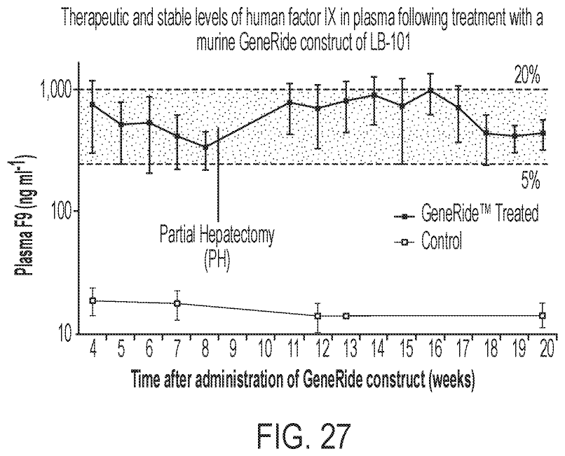

In addition, stable and therapeutic levels of human factor IX persisted for 20 weeks in neonatal wild type mice following administration of a murine GENERIDE™ construct of LB-101, even after partial hepatectomy, or, PH, as shown in FIG. 27. PH is a procedure where two-thirds of the liver is removed to trigger regenerative organ growth. With conventional AAV gene therapy, transgene expression following PH is drastically reduced.

### Example 14: Multi-Organ Diseases

Some genetic mutations result in both protein deficiencies and over-expression of deleterious proteins, leading to pathogenesis. One such disease is A1ATD. In A1ATD, patients have a deficit of circulating A1AT and can develop severe liver damage, which may necessitate a liver transplant. This is because AATD is a dominant negative genetic disease, in which the defective copy of the gene is associated with symptoms even in the presence of a normal copy. AATD is another genetic disease that has been corrected in a mouse model using a murine GENERIDE™ construct of LB-201. The GENERIDE™ construct used in the mouse model included a normal copy of the gene as well as a microRNA that was designed to reduce the expression of the deleterious gene. Expression of the transgene and downregulation of the mutant gene were evident in these mice for at least eight months.

### Example 15: Dose Response Analysis in Hemophilia B Mice

The present example demonstrates efficacy of GENERIDE™ methods to integrate Factor IX at different doses in mice.

An AAV DJ serotype was used to target human FIX-TripleL for expression after integration from the robust liver-specific mouse Alb promoter. Without wishing to be bound by any theory, it was postulated that: the Alb promoter should allow high levels of coagulation factor production even if integration takes place in only a small fraction of hepatocytes; and that the high transcriptional activity at the Alb locus should make it more susceptible to transgene integration by homologous recombination.

An in vivo gene targeting approach, based on the GENERIDE™™ technology, was applied to specifically insert a promoterless version of the therapeutic cDNA into the albumin locus, without the use of nucleases, in FIX deficient mouse models. A human FIX variant, FIX-TripleL (FIX-V86A/E277A/R338L) was used. Gene delivery of adeno-associated virus (AAV) in Hemophilia B mice showed that FIX-TripleL had 15-fold higher specific clotting activity than FIX-WT, and this activity was significantly better than FIX-Triple (10-fold) or FIX-R338L (6-fold). At a lower viral dose, FIX-TripleL improved FIX activity from sub-therapeutic to therapeutic levels. Under physiological conditions, no signs of adverse thrombotic events were observed in long-term AAV-FIX-treated C57Bl/6 mice (Kao et al. Thrombosis and Haemostasis 2013).

### Materials and Methods:

A summary of the experimental design is presented in Table 3.

TABLE 3
Summary of experimental design.

| Project | | | | | | Day of | | | Testing |
|---|---|---|---|---|---|---|---|---|---|
| # | Group | n | Age | Treatment | RoA | Sacrifice | Readout | Method | frequency |
| 01 | 1 | 3 | P2 | WT | IP | Week 12 | 1. Weight | 1. Weighing | 1. Monthly |
| | 2 | 10 | P2 | Vehicle | IP | Week 12 | 2. hFIX plasma levels | 2. ELISA | 2. Monthly |
| | 3 | 5 | P2 | hTripleL | IP | Week 12 | 3. Clotting time | 3. aPTT | 3. 4 weeks post |
| | | | | 1.5 × 10¹⁴/kg | | | | | injection |
| | 4 | 7 | P2 | hTripleL | IP | Week 12 | | | |
| | | | | 1.5 × 10¹³/kg | | | | | |
| | 5 | 11 | P2 | hTripleL | IP | Week 12 | | | |
| | | | | 1.5 × 10¹²/kg | | | | | |
| | 6 | 9 | P2 | hTripleL | IP | Week 12 | | | |
| | | | | 5 × 10¹¹/kg | | | | | |

Animal handling: Animals were housed and handled in accordance to the guidelines for animal care at both National Institute of Health (NIH) and the Association for Assessment and Accreditation of Laboratory Animal Care (AAALAC). Experimental procedures were reviewed and approved by the Israel Board for Animal Experiments. Mice were kept in a temperature-controlled environment with a 12/12 h light-dark cycle, with a standard diet and water ad libitum.

Plasmid construction: A mouse genomic Alb segment (90474003-90476720 in NCBI reference sequence: NC_000071.6) was PCR-amplified and inserted between AAV2 ITRs into BSRGI and SPEI restriction sites in a modified pTRUF backbone. The genomic segment spans 1.3 Kb upstream and 1.4 Kb downstream to the Alb stop codon. We then inserted into the BPU10I restriction site an optimized P2A coding sequence preceded by a linker coding sequence (glycine-serine-glycine) and followed by an NHEI restriction site. Finally, we inserted a codon optimized (vector NTI) hFIX-TripleL cDNA into the NHEI site to get LB-Pm-0005 (pAAV-288) that served in the construction of the DJ vector. Final rAAV production plasmids were generated using an EndoFree Plasmid Megaprep Kit (Qiagen).

AAV production: AAV-FIX-TripleL (LB-Vt-0001) vector lot #170824 (1.13E13 Total vg) was produced with CsCl purification method.

Mice injections and bleeding: F9tm1Dws knockout mice were purchased from Jackson Laboratory to serve for breeding pairs to produce offspring for neonatal injections. Two-day-old F9tm1Dws knockout males were injected intraperitoneally with 3e11, 3e10, 3e9 and 1e9 vector genomes per mouse of AAV-hFIX-TripleL and bled beginning at week 4 of life by retro-orbital bleeding for ELISA and activated partial thromboplastin time assays (using IDEXX Coag Dx Analyzer). All mice were sacrificed at week 12 and the livers were taken for DNA/Protein analysis.

FIX determination in plasma: ELISA for FIX was performed with the following antibodies; mouse anti-human FIX IgG primary antibody at 1:500 (Sigma F2645), and polyclonal goat anti-human FIX peroxidase-conjugated IgG secondary antibody at 1:4,200 (Enzyme Research GAFIX-APHRP).

Assessing rate of Alb locus targeting by LR-qPCR assay: Amplification of integrated genomic Alb, but not undesired vector amplification, was carried out using primer annealing outside the homology arm and primer for the integrated DNA, The LR-PCR amplicon served as a template for TaqMan qPCR quantification assays. We finally calculated the integration levels by standard carve of reference integrated samples.

### Results:

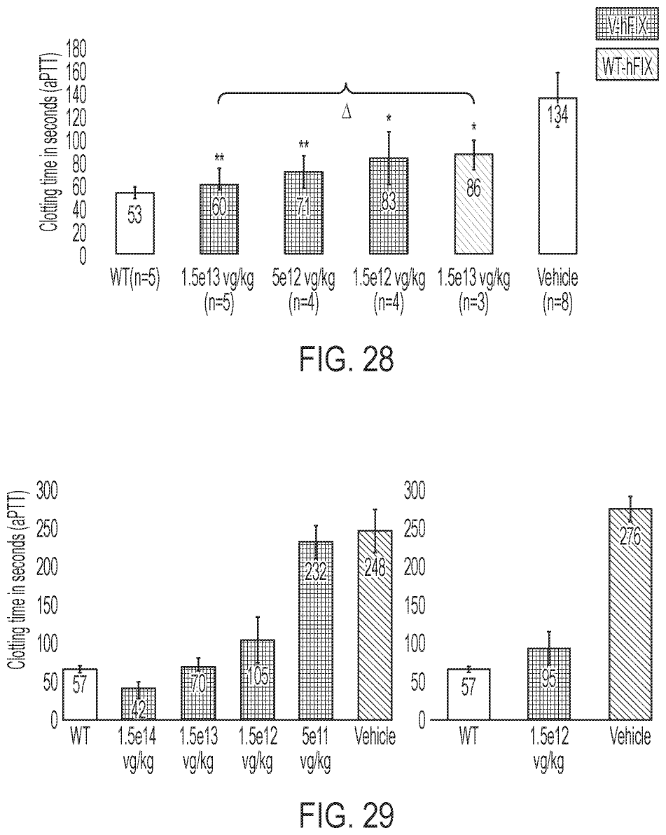

For the treatment of hemophilia B neonatal mice, Intraperitoneal (IP) injections of 2-day old F9tm1Dws knockout mice was performed with 3e11, 3e10, 3e9 and 1e9 vector genomes (vg) per mouse (1.5e14, 1.5e13, 1.5e12 and 5e11 per Kg) of an AAV-DJ GENERIDE™™ vector coding for a hyperactive variant of human FIX; FIX-TripleL. Disease amelioration was demonstrated at doses as low as 1.5E12 VG/kg. Clotting time at week 4 post injection was measured by activated partial thromboplastin time assay (aPTT). The functional coagulation, as determined by the activated partial thromboplastin time (aPTT) in treated KO mice, was restored to levels similar to that of wild-type (WT) mice (FIGS. 28-29). These results demonstrate high therapeutic hFIX-TripleL expression levels originating from on-target integration.

### Discussion:

It was observed that 1.5E12 vg/kg of hFIX-TripleL ameliorates the bleeding diathesis in hemophilia B neonates after 4 weeks and stays stable for 12 weeks. This demonstrates a therapeutic effect for in vivo gene targeting without nucleases and without a vector-borne promoter. The favorable safety profile of the disclosed promoterless and nuclease-free gene targeting strategy for rAAV makes it a prime candidate for clinical assessment in the context of hemophilia and other genetic deficiencies. More generally, this strategy could be applied whenever the therapeutic effect is conveyed by a secreted protein or when targeting confers a selective advantage.

### Example 16: Haplotype Mismatch in Homology Arms

The present example demonstrates efficacy of GENERIDE™ with mismatches in the homology arms and repeatability using different vector batches.

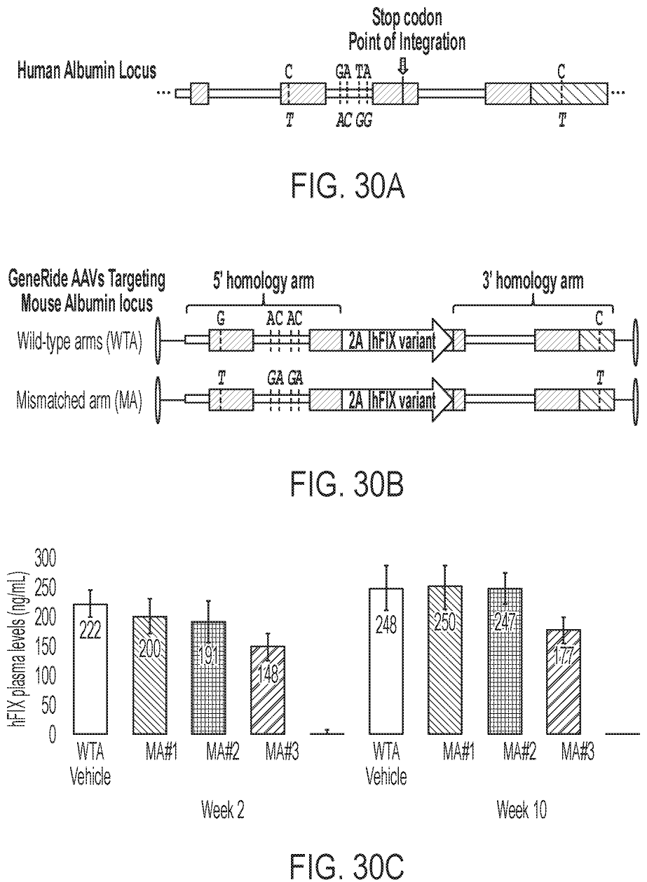

As discussed above, in GENERIDE™, the promoterless coding sequence of a therapeutic gene is targeted by natural error-free homologous recombination (HR) into the Albumin locus. The expression of the therapeutic gene is linked to the robust hepatic Albumin expression via a 2A peptide. In the relevant human Albumin locus there are 2 major haplotypes covering 95% of the population. The haplotypes differ by 5 SNPs in the sequence corresponding to the 5′ homology arm (FIG. 30A-FIG. 30C).

An AAV DJ serotype was used to target human FIX-TripleL for expression after integration from the robust liver-specific mouse Alb promoter. GENERIDE™ technology was used to specifically insert a promoterless version of the therapeutic cDNA into the albumin locus, without the use of nucleases, in Wild Type C57bl/6 mice. A wild type human FIX variant, FIX-TripleL (FIX-V86A/E277A/R338L) and a haplotype mismatch hFIX-TripleL with 6 SNPs at the homology arms were used. The haplotypes differ by 5 SNPs in the sequence corresponding to the 5′ homology arm and one SNP in the sequence corresponding to the 3′ homology arm.

### Materials and Methods:

A summary of the experimental design is presented in Table 4.

### Table 4: Summary of Experimental Design.

| | | | Vector | | | Readout | |
|---|---|---|---|---|---|---|---|
| Group | n | Age | Batch # | Treatment | RoA | Day of | Sacrifice |
| 1 | 3 | 9-week | N/A | Vehicle | IV | Week 10 | hF9 plasma levels |
| 2 | 5 | c57b1/6 | 1 | 5 × 10¹³/kg Haplotype I | | | Integration rate |
| | | Females | | (TripleL) | | | |
| 3 | 5 | | 1 | 5 × 10¹³/kg Haplotype II | | | |
| | | | | (Mutant arm) | | | |
| 4 | 5 | | 2 | 5 × 10¹³/kg Haplotype II | | | |
| | | | | (Mutant arm) | | | |
| 5 | 5 | | 3 | 5 × 10¹³/kg Haplotype II | | | |
| | | | | (Mutant arm) | | | |

Animal handling: Animals were housed and handled in accordance to the guidelines for animal care at both National Institute of Health (NIH) and the Association for Assessment and Accreditation of Laboratory Animal Care (AAALAC). Experimental procedures were reviewed and approved by the Israel Board for Animal Experiments. Mice were kept in a temperature-controlled environment with a 12/12 h light-dark cycle, with a standard diet and water ad libitum.

Plasmid construction: A mouse genomic Alb segment (90474003-90476720 in NCBI reference sequence: NC_000071.6) was PCR-amplified and inserted between AAV2 ITRs into BSRGI and SPEI restriction sites in a modified pTRUF backbone. The genomic segment spans 1.3 Kb upstream and 1.4 Kb downstream to the Alb stop codon. We then inserted into the BPU10I restriction site an optimized P2A coding sequence preceded by a linker coding sequence (glycine-serine-glycine) and followed by an NHEI restriction site. Finally, we inserted a codon optimized (vector NTI) hFIX-TripleL cDNA into the NHEI site to get LB-Pm-0005 (pAAV-288) that served in the construction of the DJ vector. Final rAAV production plasmids were generated using an EndoFree Plasmid Megaprep Kit (Qiagen).

AAV production: AAV-FIX-TripleL (LB-Vt-0001) vector lot #171102 was serve as positive control and three different vector batches of Haplotype mismatch lots #171102, 171116, 171130 produced with CsCl purification method.

Mice injections and bleeding: Nine-week-old C57bl/6 female mice were injected intraperitoneally with 1e12 vector genomes per mouse of AAV-hFIX-TripleL w/o mismatches and bled Two, Four, Seven and Ten weeks post-injection by retro-orbital bleeding for protein level measurements by ELISA. All mice were sacrificed at week 10 and the livers were taken for DNA integration rate analysis.

FIX determination in plasma: ELISA for FIX was performed with the following antibodies; mouse anti-human FIX IgG primary antibody at 1:500 (Sigma F2645), and polyclonal goat anti-human FIX peroxidase-conjugated IgG secondary antibody at 1:4,200 (Enzyme Research GAFIX-APHRP).

Assessing rate of Alb locus targeting by LR-qPCR assay: Amplification of integrated genomic Alb, but not undesired vector amplification, was carried out using primer annealing outside the homology arm and primer for the integrated DNA, The LR-PCR amplicon served as a template for TaqMan qPCR quantification assays. We finally calculated the integration levels by standard carve of reference integrated samples.

### Results:

For the treatment of C57bl/6 adult mice, Intravenous (IV) injections of 9-week old C57bl/6 mice were performed with 1e12 vector genomes (VG) per mouse (5e13 per Kg) of an AAV-DJ GENERIDE™™ vector coding for a hyperactive variant of human FIX; FIX-TripleL w/o mismatches. Vectors with synthetic mouse haplotypes baring analogous mutations were designed and it was found that GENERIDE™™ is largely unaffected by this haplotype mismatch. This observation supports the ability to use one vector design for different populations of patients. High consistency was found between the different vectors produced independently and separately. A stable presence of hFIX protein in the plasma along 10 weeks was observed.

### Discussion:

Previous results demonstrated amelioration of the bleeding diathesis in hemophilia B mice after a single injection to either adult or neonatal mice of 1.5e12 vg/kg of a GENERIDE™™ vector coding for hFIX-TripleL variant. In this study, it was shown that GENERIDE™™ efficiency is not reduced by mismatches between the homology arms on the vector and the target locus when the mismatches simulate common human haplotypes. This work also demonstrated robust and consistent vector production capabilities. The favorable efficacy and safety profile of the promoterless and nuclease-free gene targeting strategy for rAAV makes GENERIDE™™ a prime candidate for clinical assessment in the context of hemophilia and other genetic deficiencies. This therapeutic effect can be achieved with one vector design that can be suitable for all population.

### Example 17: Capsids for Applications in GENERIDE™ Technology

The present example provides exemplary capsids that can be used in applications of the GENERIDE™ technology. Exemplary capsids that can be used for transgene expression using GENERIDE™ include AAV8, AAV-DJ, LK03, and NP59.

SEQ ID NO: 1 is the amino acid sequence of the capsid protein of AAV-DJ. SEQ ID NO: 2 is a nucleotide sequence encoding the capsid protein of AAV-DJ. Additional information on AAV-DJ can be found in WO/2007/120542, incorporated herein by reference.

SEQ ID NO: 5 is a nucleotide sequence encoding the capsid protein of AAV-LK03. SEQ ID NO: 6 is the amino acid sequence of the capsid protein of AAV-LK03. Additional information on LK03 can be found in WO/2013/029030, incorporated herein by reference.

SEQ ID NO: 7 is a nucleotide sequence encoding the capsid protein of AAV-NP59. SEQ ID NO: 8 is the amino acid sequence of the capsid protein of AAV-NP59. Additional information on NP59 can be found in WO/2017/143100, incorporated herein by reference.

### Example 18: Continued Evolution of the GENERIDE™ Platform

Key aspects of the GENERIDE™ platform from the design of the constructs and capsids to manufacturing at a commercial scale can be optimized.
AAV capsid. AAV capsids are designed to be highly efficient in delivering their contents to specific target tissues such as the liver. Capsids have been identified that are better suited for clinical use in the liver and other indications. For example, LK03, the AAV capsid used in LB-001, was developed to be liver selective.
Homology arms and integration sites. Genome editing technology has the potential advantage that the homology arms and integration sites for one therapy can be applied to other therapies that target the same tissue. Insight gained from optimization of the rate of homologous recombination and gene expression levels can be applied to subsequent product candidates.
Targets. Potential targets include those that correspond to genes normally expressed in the liver, other tissues related to liver expression, and targets that are best addressed directly in other tissues such as the CNS or muscle.
Selection. A potential advantage of the GENERIDE™ genome editing technology is its durable nature arising from chromosomal integration. Data indicates that there are therapies where correction of a gene deficiency may provide a selective advantage to cells and drive expansion of the percentage of cells containing the transgene. Methods of providing a selective advantage to treated cells even when the transgene does not provide a selection advantage at the cellular level are also being evaluated. One such method involves adding an element to a GENERIDE™ construct such that cells that do not incorporate the element are at a selective disadvantage when patients are treated with an external agent. These and related methods will enable enrichment of the number of cells containing the desired gene ensuring that patients derive long-term therapeutic benefit.

SEQUENCES
SEQ ID NO: 1 is the amino acid sequence of the capsid protein of AAV-DJ.
MAADGYLPDWLEDTLSEGIRQWWKLKPGPPPPKPAERHKDDSRGLVLPGYKYLGPFNG
LDKGEPVNEADAAALEHDKAYDRQLDSGDNPYLKYNHADAEFQERLKEDTSFGGNLG
RAVFQAKKRLLEPLGLVEEAAKTAPGKKRPVEHSPVEPDSSSGTGKAGQQPARKRLNF
GQTGDADSVPDPQPIGEPPAAPSGVGSLTMAAGGGAPMADNNEGADGVGNSSGNWHC
DSTWMGDRVITTSTRTWALPTYNNHLYKQISNSTSGGSSNDNAYFGYSTPWGYFDFNR
FHCHFSPRDWQRLINNNWGFRPKRLSFKLFNIQVKEVTQNEGTKTIANNLTSTIQVFTDS
EYQLPYVLGSAHQGCLPPFPADVFMIPQYGYLTLNNGSQAVGRSSFYCLEYFPSQMLKT
GNNFQFTYTFEDVPFHSSYAHSQSLDRLMNPLIDQYLYYLSRTQTTGGTTNTQTLGFSQ
GGPNTMANQAKNWLPGPCYRQQRVSKTSADNNNSEYSWTGATKYHLNGRDSLVNPG
PAMASHKDDEEKFFPQSGVLIFGKQGSEKTNVDIEKVMITDEEEIRTTNPVATEQYGSVS
TNLQRGNRQAATADVNTQGVLPGMVWQDRDVYLQGPIWAKIPHTDGHFHPSPLMGGF
GLKHPPPQILIKNTPVPADPPTTFNQSKLNSFITQYSTGQVSVEIEWELQKENSKRWNPEI
QYTSNYYKSTSVDFAVNTEGVYSEPRPIGTRYLTRNL
SEQ ID NO: 2 is a nucleotide sequence encoding the capsid protein of AAV-DJ.
atggctgccgatggttatcttccagattggctcgaggacactctctctgaaggaataagacagtggtggaagctcaaacctggcccaccacc
accaaagcccgcagagcggcataaggacgacagcaggggtcttgtgcttcctgggtacaagtacctaggacccttcaacggactcgaca
agggagagccggtcaacgaggcagacgccgcggccctcgagcacgacaaagcctacgaccggcagctcgacagcggagacaaccc
gtacctcaagtacaaccacgccgacgccgagttccaggagaggctcaaagaagatacgtcttttgggggcaacctcgggcgagcagtctt
ccaggccaaaaagaggcttcttgaacctcttggtctggttgaggaagcggctaagacggctcctggaaagaagaggcctgtagagcactct
cctgtggagccagactcctcctcgggaaccggaaaggcgggccagcagcctgcaagaaaaagattgaattttggtcagactggagacgc
agactcagtcccagaccctcaaccaatcggagaacctcccgcagccccctcaggtgtgggatctcttacaatggctgcaggcggtggcgc
accaatggcagacaataacgagggcgccgacggagtgggtaattcctcgggaaattggcattgcgattccacatggatgggcgacagagt
catcaccaccagcacccgaacctgggccctgcccacctacaacaaccacctctacaagcaaatctccaacagcacatctggaggatcttca
aatgacaacgcctacttcggctacagcaccccctgggggtattttgactttaacagattccactgccacttttcaccacgtgactggcagcg
actcatcaacaacaactggggattccggcccaagagactcagcttcaagctcttcaacatccaggtcaaggaggtcacgcagaatgaaggca
ccaagaccatcgccaataacctcaccagcaccatccaggtgtttacggactcggagtaccagctgccgtacgttctcggctctgcccacca
gggctgcctgcctccgttcccggcggacgtgttcatgattccccagtacggctacctaacactcaacaacggtagtcaggccgtgggacgc
tcctccttctactgcctggaatactttccttcgcagatgctgagaaccggcaacaacttccagtttacttacaccttcgaggacgtgccttt
ccacagcagctacgcccacagccagagcttggaccggctgatgaatcctctgattgaccagtacctgtactacttgtctcggactcaaacaa
caggaggcacgacaaatacgcagactctgggcttcagccaaggtgggcctaatacaatggccaatcaggcaaagaactggctgccaggaccct
gttaccgccagcagcgagtatcaaagacatctgcggataacaacaacagtgaatactcgtggactggagctaccaagtaccacctcaatgg
cagagactctctggtgaatccgggcccggccatggcaagccacaaggacgatgaagaaaagtttttttcctcagagcggggttctcatcttt
gggaagcaaggctcagagaaaacaaatgtggacattgaaaaggtcatgattacagacgaagaggaaatcaggacaaccaatcccgtggc
tacggagcagtatggttctgtatctaccaacctccagagaggcaacagacaagcagctaccgcagatgtcaacacacaaggcgttcttcca
ggcatggtctggcaggacagagatgtgtaccttcaggggcccatctgggcaaagattccacacacggacggacattttcacccctctcccc
tcatgggtggattcggacttaaacaccctccgcctcagatcctgatcaagaacacgcctgtacctgcggatcctccgaccaccttcaaccagt
caaagctgaactctttcatcacccagtattctactggccaagtcagcgtggagatcgagtgggagctgcagaaggaaaacagcaagcgctg
gaaccccgagatccagtacacctccaactactacaaatctacaagtgtggactttgctgttaatacagaaggcgtgtactctgaaccccgccc
cattggcacccgttacctcacccgtaatctgtaa
SEQ ID NO: 3 is the amino acid sequence of the capsid protein of AAV-2.
MAADGYLPDWLEDTLSEGIRQWWKLKPGPPPPKPAERHKDDSRGLVLPGYKYLGPFNG
LDKGEPVNEADAAALEHDKAYDRQLDSGDNPYLKYNHADAEFQERLKEDTSFGGNLG
RAVFQAKKRVLEPLGLVEEPVKTAPGKKRPVEHSPVEPDSSSGTGKAGQQPARKRLNFG
QTGDADSVPDPQPLGQPPAAPSGLGTNTMATGSGAPMADNNEGADGVGNSSGNWHCD
STWMGDRVITTSTRTWALPTYNNHLYKQISSQSGASNDNHYFGYSTPWGYFDFNRFHC
HFSPRDWQRLINNNWGFRPKRLNFKLFNIQVKEVTQNDGTTTIANNLTSTVQVFTDSEY
QLPYVLGSAHQGCLPPFPADVFMVPQYGYLTLNNGSQAVGRSSFYCLEYFPSQMLRTG
NNFTFSYTFEDVPFHSSYAHSQSLDRLMNPLIDQYLYYLSRTNTPSGTTTQSRLQFSQAG
ASDIRDQSRNWLPGPCYRQQRVSKTSADNNNSEYSWTGATKYHLNGRDSLVNPGPAM
ASHKDDEEKFFPQSGVLIFGKQGSEKTNVDIEKVMITDEEEIRTTNPVATEQYGSVSTNL
QRGNRQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTS
NYNKSVNRGLTVDTNGVYSEPRPIGTRYLTRNL
SEQ ID NO: 4 is the amino acid sequence of the capsid protein of AAV-8.
MAADGYLPDWLEDNLSEGIREWWALKPGAPKPKANQQKQDDGRGLVLPGYKYLGPF
NGLDKGEPVNAADAAALEHDKAYDQQLQAGDNPYLRYNHADAEFQERLQEDTSFGGN
LGRAVFQAKKRVLEPLGLVEEGAKTAPGKKRPVEPSPQRSPDSSTGIGKKGQQPARKRL
NFGQTGDSESVPDPQPLGEPPAAPSGVGPNTMAAGGGAPMADNNEGADGVGSSSGNW
HCDSTWLGDRVITTSTRTWALPTYNNHLYKQISNGTSGGATNDNTYFGYSTPWGYFDF
NRFHCHFSPRDWQRLINNNWGFRPKRLSFKLFNIQVKEVTQNEGTKTIANNLTSTIQVFT
DSEYQLPYVLGSAHQGCLPPFPADVFMIPQYGYLTLNNGSQAVGRSSFYCLEYFPSQML
RTGNNFQFTYTFEDVPFHSSYAHSQSLDRLMNPLIDQYLYYLSRTQTTGGTANTQTLGF
SQGGPNTMANQAKNWLPGPCYRQQRVSTTTGQNNNSNFAWTAGTKYHLNGRNSLAN
PGIAMATHKDDEERFFPSNGILIFGKQNAARDNADYSDVMLTSEEEIKTTNPVATEEYGI
VADNLQQQNTAPQIGTVNSQGALPGMVWQNRDVYLQGPIWAKIPHTDGNFHPSPLMG
GFGLKHPPPQILIKNTPVPADPPTTFNQSKLNSFITQYSTGQVSVEIEWELQKENSKRWNP
EIQYTSNYYKSTSVDFAVNTEGVYSEPRPIGTRYLTRNL
SEQ ID NO: 5 is a nucleotide sequence encoding the capsid protein of AAV-LK03.
atggctgctgacggttatcttccagattggctcgaggacaacctttctgaaggcattcgagagtggtgggcgctgcaacctggagcccctaa
acccaaggcaaatcaacaacatcaggacaacgctcggggtcttgtgcttccgggttacaaatacctcggacccggcaacggactcgacaa
gggggaacccgtcaacgcagcggacgcggcagccctcgagcacgacaaggcctacgaccagcagctcaaggccggtgacaacccct
acctcaagtacaaccacgccgacgccgagttccaggagcggctcaaagaagatacgtcttttgggggcaacctcgggcgagcagtcttcc
aggccaaaaagaggcttcttgaacctcttggtctggttgaggaagcggctaagacggctcctggaaagaagaggcctgtagatcagtctcc
tcaggaaccggactcatcatctggtgttggcaaatcgggcaaacagcctgccagaaaaagactaaatttcggtcagactggcgactcagag
tcagtcccagaccctcaacctctcggagaaccaccagcagcccccacaagtttgggatctaatacaatggcttcaggcggtggcgcacca
atggcagacaataacgagggtgccgatggagtgggtaattcctcaggaaattggcattgcgattcccaatggctgggcgacagagtcatca
ccaccagcaccagaacctgggccctgcccacttacaacaaccatctctacaagcaaatctccagccaatcaggagcttcaaacgacaacc
actactttggctacagcaccccttgggggtattttgactttaacagattccactgccacttctcaccacgtgactggcagcgactcattaaca
acaactggggattccggcccaagaaactcagcttcaagctcttcaacatccaagttaaagaggtcacgcagaacgatggcacgacgactattg
ccaataaccttaccagcacggttcaagtgtttacggactcggagtatcagctcccgtacgtgctcgggtcggcgcaccaaggctgtctcccg
ccgtttccagcggacgtcttcatggtccctcagtatggatacctcaccctgaacaacggaagtcaagcggtgggacgctcatccttttactgc
ctggagtacttcccttcgcagatgctaaggactggaaataacttccaattcagctataccttcgaggatgtaccttttcacagcagctacgctc
acagccagagtttggatcgcttgatgaatcctcttattgatcagtatctgtactacctgaacagaacgcaaggaacaacctctggaacaaccaa
ccaatcacggctgctttttagccaggctgggcctcagtctatgtctttgcaggccagaaattggctacctgggccctgctaccggcaacaga
gactttcaaagactgctaacgacaacaacaacagtaactttccttggacagcggccagcaaatatcatctcaatggccgcgactcgctggtg
aatccaggaccagctatggccagtcacaaggacgatgaagaaaaatttttccctatgcacggcaatctaatatttggcaaagaagggacaac
ggcaagtaacgcagaattagataatgtaatgattacggatgaagaagagattcgtaccaccaatcctgtggcaacagagcagtatggaact
gtggcaaataacttgcagagctcaaatacagctcccacgactagaactgtcaatgatcagggggccttacctggcatggtgtggcaagatc
gtgacgtgtaccttcaaggacctatctgggcaaagattcctcacacggatggacactttcatccttctcctctgatgggaggctttggactgaa
acatccgcctcctcaaatcatgatcaaaaatactccggtaccggcaaatcctccgacgactttcagcccggccaagtttgcttcatttatcact
cagtactccactggacaggtcagcgtggaaattgagtgggagctacagaaagaaaacagcaaacgttggaatccagagattcagtacacttc
caactacaacaagtctgttaatgtggactttactgtagacactaatggtgtttatagtgaacctcgccccattggcacccgttaccttacccgt
cccctgtaa
SEQ ID NO: 6 is the amino acid sequence of the capsid protein of AAV-LK03.
MAADGYLPDWLEDNLSEGIREWWALQPGAPKPKANQQHQDNARGLVLPGYKYLGPG
NGLDKGEPVNAADAAALEHDKAYDQQLKAGDNPYLKYNHADAEFQERLKEDTSFGG
NLGRAVFQAKKRLLEPLGLVEEAAKTAPGKKRPVDQSPQEPDSSSGVGKSGKQPARKR
LNFGQTGDSESVPDPQPLGEPPAAPTSLGSNTMASGGGAPMADNNEGADGVGNSSGNW
HCDSQWLGDRVITTSTRTWALPTYNNHLYKQISSQSGASNDNHYFGYSTPWGYFDENR
FHCHFSPRDWQRLINNNWGFRPKKLSFKLFNIQVKEVTQNDGTTTIANNLTSTVQVFTD
SEYQLPYVLGSAHQGCLPPFPADVFMVPQYGYLTLNNGSQAVGRSSFYCLEYFPSQMLR
TGNNFQFSYTFEDVPFHSSYAHSQSLDRLMNPLIDQYLYYLNRTQGTTSGTTNQSRLLFS
QAGPQSMSLQARNWLPGPCYRQQRLSKTANDNNNSNFPWTAASKYHLNGRDSLVNPG
PAMASHKDDEEKFFPMHGNLIFGKEGTTASNAELDNVMITDEEEIRTTNPVATEQYGTV
ANNLQSSNTAPTTRTVNDQGALPGMVWQDRDVYLQGPIWAKIPHTDGHFHPSPLMGGF
GLKHPPPQIMIKNTPVPANPPTTFSPAKFASFITQYSTGQVSVEIEWELQKENSKRWNPEI
QYTSNYNKSVNVDFTVDTNGVYSEPRPIGTRYLTRPL
SEQ ID NO: 7 is a nucleotide sequence encoding the capsid protein of AAV-NP59.
atggctgccgatggttatcttccagattggctcgaggacactctctctgaaggaataagacagtggtggaagctcaaacctggcccaccacc
accaaagcccgcagagcggcataaggacgacagcaggggtcttgtgcttcctgggtacaagtacctcggacccttcaacggactcgaca
agggagagccggtcaacgaggcagacgccgcggccctcgagcacgacaaagcctacgaccggcagctcgacagcggagacaaccc
gtacctcaagtacaaccacgccgacgcggagtttcaggagcgccttaaagaagatacgtcttttgggggcaacctcggacgagcagtcttc
caggcgaaaaagagggttcttgaacctctgggcctggttgaggaacctgttaagacggctccgggaaaaaagaggccggtagagcactct
cctgtggagccagactcctcctcgggaaccggcaagacaggccagcagcccgctaaaaagagactcaattttggtcagactggcgactca
gagtcagtcccagaccctcaacctctcggagaaccaccagcagccccctctggtctgggaactaatacgatggctacaggcagtggcgca
ccaatggcagacaataacgagggcgccgacggagtgggtaattcctcgggaaattggcattgcgattccacatggatgggcgacagagtc
atcaccaccagcacccgaacctgggccctgcccacctacaacaaccatctctacaagcaaatctccagccaatcaggagcttcaaacgac
aaccactactttggctacagcaccccttgggggtattttgactttaacagattccactgccacttctcaccacgtgactggcagcgactcatt
aacaacaactggggattccggcccaagaaactcagcttcaagctcttcaacatccaagttaaagaggtcacgcagaacgatggcacgacgac
tattgccaataaccttaccagcacggttcaagtgtttactgactcggagtaccagctcccgtacgtcctcggctcggcgcatcaaggatgcct
cccgccgttcccagcagacgtcttcatggtgccacagtatggatacctcaccctgaacaacgggagtcaggcagtaggacgctcttcatttt
actgcctggagtactttccttctcagatgctgcgtaccggaaacaactttaccttcagctacacttttgaggacgttcctttccacagcagcta
cgctcacagccagagtctggaccgtctcatgaatcctctcatcgaccagtacctgtattacttgagcagaacaaacactccaagtggaaccacc
acgcagtcaaggcttcagttttctcaggccggagcgagtgacattcgggaccagtctaggaactggcttcctggaccctgttaccgccagca
gcgagtatcaaagacatctgcggataacaacaacagtgaatactcgtggactggagctaccaagtaccacctcaatggcagagactctctg
gtgaatccgggcccggccatggcaagccacaaggacgatgaagaaaagttttttcctcagagcggggttctcatctttgggaagcaaggct
cagagaaaacaaatgtggacattgaaaaggtcatgattacagacgaagaggaaatcaggacaaccaatcccgtggctacggagcagtatg
gttctgtatctaccaacctccagagaggcaacagacaagcagctaccgcagatgtcgacacacaaggcgttcttccaggcatggtctggca
ggacagagatgtgtaccttcagggacccatctgggcaaagattccacacacggacggacattttcacccctctcccctcatgggtggattcg
gacttaaacaccctcctccacagattctcatcaagaacaccccggtacctgcgaatccttcgaccaccttcagtgcggcaaagtttgcttcctt
catcacacagtactccacgggacaggtcagcgtggagatcgagtgggagctgcagaaggaaaacagcaaacgctggaatcccgaaattc
agtacacttccaactacaacaagtctgttaatgtggactttactgtggacactaatggcgtgtattcagagcctcgccccattggcaccagata
cctgactcgtaatctgtaa
SEQ ID NO: 8 is the amino acid sequence of the capsid protein of AAV-NP59.
MAADGYLPDWLEDTLSEGIRQWWKLKPGPPPPKPAERHKDDSRGLVLPGYKYLGPFNG
LDKGEPVNEADAAALEHDKAYDRQLDSGDNPYLKYNHADAEFQERLKEDTSFGGNLG
RAVFQAKKRVLEPLGLVEEPVKTAPGKKRPVEHSPVEPDSSSGTGKTGQQPAKKRLNFG
QTGDSESVPDPQPLGEPPAAPSGLGTNTMATGSGAPMADNNEGADGVGNSSGNWHCD
STWMGDRVITTSTRTWALPTYNNHLYKQISSQSGASNDNHYFGYSTPWGYFDFNRFHC
HFSPRDWQRLINNNWGFRPKKLSFKLFNIQVKEVTQNDGTTTIANNLTSTVQVFTDSEY
QLPYVLGSAHQGCLPPFPADVFMVPQYGYLTLNNGSQAVGRSSFYCLEYFPSQMLRTG
NNFTFSYTFEDVPFHSSYAHSQSLDRLMNPLIDQYLYYLSRTNTPSGTTTQSRLQFSQAG
ASDIRDQSRNWLPGPCYRQQRVSKTSADNNNSEYSWTGATKYHLNGRDSLVNPGPAM
ASHKDDEEKFFPQSGVLIFGKQGSEKTNVDIEKVMITDEEEIRTTNPVATEQYGSVSTNL
QRGNRQAATADVDTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTS
NYNKSVNVDFTVDTNGVYSEPRPIGTRYLTRNL
SEQ ID NO: 9 is an optimized nucleotide sequence encoding human methylmalonyl-CoA
mutase (synMUT1)
atgctgagagccaaaaaccagctgttcctgctgagcccccactatctgagacaggtcaaagaaagttccgggagtagactgatccagcag
agactgctgcaccagcagcagccactgcatcctgagtgggccgctctggccaagaaacagctgaagggcaaaaacccagaagacctga
tctggcacactccagaggggatttcaatcaagcccctgtacagcaaaagggacactatggatctgccagaggaactgccaggagtgaagc
ctttcacccgcggaccttacccaactatgtatacctttcgaccctggacaattcggcagtacgccggcttcagtactgtggaggaatcaaaca
agttttataaggacaacatcaaggctggacagcagggcctgagtgtggcattcgatctggccacacatcgcggctatgactcagataatccc
agagtcaggggggacgtgggaatggcaggagtcgctatcgacacagtggaagatactaagattctgttcgatggaatccctctggagaaa
atgtctgtgagtatgacaatgaacggcgctgtcattcccgtgctggcaaacttcatcgtcactggcgaggaacagggggtgcctaaggaaa
aactgaccggcacaattcagaacgacatcctgaaggagttcatggtgcggaatacttacatttttccccctgaaccatccatgaaaatcattgc
cgatatcttcgagtacaccgctaagcacatgcccaagttcaactcaattagcatctccgggtatcatatgcaggaagcaggagccgacgcta
ttctggagctggcttacaccctggcagatggcctggaatattctcgaaccggactgcaggcaggcctgacaatcgacgagttcgctcctaga
ctgagtttcttttggggaattggcatgaacttttacatggagatcgccaagatgagggctggccggagactgtgggcacacctgatcgagaa
gatgttccagcctaagaactctaagagtctgctgctgcgggcccattgccagacatccggctggtctctgactgaacaggacccatataaca
atattgtcagaaccgcaatcgaggcaatggcagccgtgttcggaggaacccagagcctgcacacaaactcctttgatgaggccctggggc
tgcctaccgtgaagtctgctaggattgcacgcaatacacagatcattatccaggaggaatccggaatcccaaaggtggccgatccctgggg
aggctcttacatgatggagtgcctgacaaacgacgtgtatgatgctgcactgaagctgattaatgaaatcgaggaaatggggggaatggca
aaggccgtggctgagggcattccaaaactgaggatcgaggaatgtgcagctaggcgccaggcacgaattgactcaggaagcgaagtgat
cgtcggggtgaataagtaccagctggagaaagaagacgcagtcgaagtgctggccatcgataacacaagcgtgcgcaatcgacagattg
agaagctgaagaaaatcaaaagctcccgcgatcaggcactggccgaacgatgcctggcagccctgactgagtgtgctgcaagcgggga
cggaaacattctggctctggcagtcgatgcctcccgggctagatgcactgtgggggaaatcaccgacgccctgaagaaagtcttcggaga
gcacaaggccaatgatcggatggtgagcggcgcttatagacaggagttcggggaatctaaagagattaccagtgccatcaagagggtgca
caagttcatggagagagaagggcgacggcccaggctgctggtggcaaagatgggacaggacggacatgatcgcggagcaaaagtcatt
gccaccgggttcgctgacctgggatttgacgtggatatcggccctctgttccagacaccacgagaggtcgcacagcaggcagtcgacgct
gatgtgcacgcagtcggagtgtccactctggcagctggccataagaccctggtgcctgaactgatcaaagagctgaactctctgggcagac
cagacatcctggtcatgtgcggcggcgtgatcccaccccaggattacgaattcctgtttgaggtcggggtgagcaacgtgttcggaccagg
aaccaggatccctaaggccgcagtgcaggtcctggatgatattgaaaagtgtctggaaaagaaacagcagtcagtgtaa
SEQ ID NO: 10 is the naturally occurring (wt) amino acid sequence of human
methylmalonyl-CoA mutase.
MLRAKNQLFLLSPHYLRQVKESSGSRLIQQRLLHQQQPLHPEWAALAKKQLKGKNPED
LIWHTPEGISIKPLYSKRDTMDLPEELPGVKPFTRGPYPTMYTFRPWTIRQYAGFSTVEES
NKFYKDNIKAGQQGLSVAFDLATHRGYDSDNPRVRGDVGMAGVAIDTVEDTKILFDGI
PLEKMSVSMTMNGAVIPVLANFIVTGEEQGVPKEKLTGTIQNDILKEFMVRNTYIEPPEP
SMKIIADIFEYTAKHMPKENSISISGYHMQEAGADAILELAYTLADGLEYSRTGLQAGLTI
DEFAPRLSFFWGIGMNFYMEIAKMRAGRRLWAHLIEKMFQPKNSKSLLLRAHCQTSGW
SLTEQDPYNNIVRTAIEAMAAVFGGTQSLHTN SFDEALGLPTVKSARIARNTQIIIQEESGI
PKVADPWGGSYMMECLTNDVYDAALKLINEIEEMGGMAKAVAEGIPKLRIEECAARRQ
ARIDSGSEVIVGVNKYQLEKEDAVEVLAIDNTSVRNRQIEKLKKIKSSRDQALAEHCLAA
LTECAASGDGNILALAVDASRARCTVGEITDALKKVFGEHKANDRMVSGAYRQEFGES
KEITSAIKRVHKFMEREGRRPRLLVAKMGQDGHDRGAKVIATGFADLGEDVDIGPLFQT
PREVAQQAVDADVHAVGVSTLAAGHKTLVPELIKELNSLGRPDILVMCGGVIPPQDYEF
LFEVGVSNVFGPGTRIPKAAVQVLDDIEKCLEKKQQSV
SEQ ID NO: 11 is the naturally-occurring (wt) nucleotide sequence human methylmalonyl-
CoA mutase gene (wtMUT).
atgttaagagctaagaatcagctttttttactttcacctcattacctgaggcaggtaaaagaatcatcaggctccaggctcatacagcaacga
cttctacaccagcaacagccccttcacccagaatgggctgccctggctaaaaagcagctgaaaggcaaaaacccagaagacctaatatggca
caccccggaagggatctctataaaacccttgtattccaagagagatactatggacttacctgaagaacttccaggagtgaagccattcacac
gtggaccatatcctaccatgtatacctttaggccctggaccatccgccagtatgctggttttagtactgtggaagaaagcaataagttctataa
ggacaacattaaggctggtcagcagggattatcagttgcctttgatctggcgacacatcgtggctatgattcagacaaccdcgagttcgtggt
gatgttggaatggctggagttgctattgacactgtggaagataccaaaattctttttgatggaattcctttagaaaaaatgtcagtttccatg
actatgaatggagcagttattccagttcttgcaaattttatagtaactggagaagaacaaggtgtacctaaagagaagcttactggtaccatc
caaaatgatatactaaaggaatttatggttcgaaatacatacatttttcctccagaaccatccatgaaaattattgctgacatatttgaatat
acagcaaagcacatgccaaaatttaattcaatttcaattagtggataccatatgcaggaagcaggggctgatgccattctggagctggcctat
actttagcagatggattggagtactctagaactggactccaggctggcctgacaattgatgaatttgcaccaaggttgtctttcttctgggga
attggaatgaatttctatatggaaatagcaaagatgagagctggtagaagactctgggctcacttaatagagaaaatgtttcagcctaaaaac
tcaaaatctcttcttctaagagcacactgtcagacatctggatggtcacttactgagcaggatccctacaataatattgtccgtactgcaata
gaagcaatggcagcagtatttggagggactcagtctttgcacacaaattcttttgatgaagctttgggtttgccaactgtgaaaagtgctcga
attgccaggaacacacaaatcatcattcaagaagaatctgggattcccaaagtggctgatccttggggaggttcttacatgatggaatgtctc
acaaatgatgtttatgatgctgctttaaagctcattaatgaaattgaagaaatgggtggaatggccaaagctgtagctgagggaatacctaaa
cttcgaattgaagaatgtgctgcccgaagacaagctagaatagattctggttctgaagtaattgttggagtaaataagtaccagttggaaaaa
gaagacgctgtagaagttctggcaattgataatacttcagtgcgaaacaggcagattgaaaaacttaagaagatcaaatccagcagggatcaa
gctttggctgaacgttgtcttgctgcactaaccgaatgtgctgctagcggagatggaaatatcctggctcttgcagtggatgcatctcgggca
agatgtacagtgggagaaatcacagatgccctgaaaaaggtatttggtgaacataaagcgaatgatcgaatggtgagtggagcatatcgccag
gaatttggagaaagtaaagagataacatctgctatcaagagggttcataaattcatggaacgtgaaggtcgcagacctcgtcttcttgtagca
aaaatgggacaagatggccatgacagaggagcaaaagttattgctacaggatttgctgatcttggttttgatgtggacataggccctcttttc
cagactcctcgtgaagtggcccagcaggctgtggatgcggatgtgcatgctgtgggcataagcaccctcgctgctggtcataaaaccctagtt
cctgaactcatcaaagaacttaactcccttggacggccagatattcttgtcatgtgtggaggggtgataccacctcaggattatgaatttctg
tttgaagttggtgtttccaatgtatttggtcctgggactcgaattccaaaggctgccgttcaggtgcttgatgatattgagaagtgtttggaa
aagaagcagcaatctgtataa
SEQ ID NO: 12 is an optimized nucleotide sequence encoding human methylmalonyl-CoA
mutase (synMUT2)
atgctgcgagcgaaaaatcagctttttctgttgagcccacactacctgaggcaggttaaagaatccagcgggagccggctgattcagcagc
gactgctccaccagcagcagcctttgcatcccgaatgggctgctttggcgaagaagcagctcaaggggaagaaccctgaagatcttatttg
gcacaccccagagggcatcagcatcaagcctttgtattccaaaagggacaccatggatctgcctgaagaattgcccggggtcaaaccattc
acacgggggccatatccaaccatgtacaccttccggccatggactatcagacagtatgcaggctttagcactgtcgaggaatccaataagtt
ctataaagacaatatcaaagctggccagcaaggtctgtccgtggcattcgatctggctacacatagaggttatgattctgacaatccaagagt
acggggagacgtcggaatggcgggagttgccattgacacagtggaggacaccaagatacttttcgatgggattccattggagaaaatgtct
gtgtcaatgacgatgaacggcgctgtgattcccgttttggcgaacttcatcgtcaccggggaagagcagggcgtcccgaaggaaaagctc
accgggacaatccaaaacgacattcttaaagaattcatggtgagaaatacctacatctttcctcctgagccttccatgaagatcatcgcggaca
tctttgaatacacggctaaacacatgcctaaatttaactcaatcagcataagcgggtaccacatgcaggaggccggcgctgacgctatacttg
agctcgcatataccctggcagatggactggaatactcaaggaccgggctccaggctggactgacaatcgacgagtttgccccccgactca
gttttttctggggtatcgggatgaatttctacatggagatagcgaagatgagggcgggcagacggctttgggcgcatctgatcgagaaaatgt
tccagcccaagaattcaaagagtctgctgctgagagcccactgccagacctcaggctggagcctgactgaacaggacccatacaacaaca
ttgttagaaccgccatcgaggcgatggcagcggttttcggtgggacacagtcattgcacactaactcatttgacgaagccdcggtctgccta
ccgtgaagtcagctcggatcgctaggaacacacagatcatcatccaggaggagagtggcatcccaaaagtcgccgatccttggggagga
agttacatgatggaatgcctcacgaatgacgtatacgatgccgcactcaagctgattaacgagatcgaggaaatgggaggcatggcaaaa
gctgtcgccgagggcattccaaagctgcgcatagaggagtgtgccgcccgaagacaggcccgcattgactccggctctgaggtgatagt
gggcgttaataaatatcagctagagaaggaagacgccgtcgaagttctggcgatagataatacctctgtgcgaaatagacagattgagaaa
ctgaagaagatcaagtcaagccgagaccaggccttggccgagaggtgtctggcagccctcactgagtgcgcggcatctggggacggca
acatattggcacttgccgtcgatgcctccagggcccgatgtacggtcggcgaaattaccgatgccctcaagaaggtttttggcgagcacaag
gctaacgacaggatggttagtggagcatacagacaggagtttggcgaaagcaaggaaattacttccgcgattaaaagagtgcacaaattca
tggaacgggagggtaggcgaccgaggctcctcgttgccaaaatgggtcaggacggccacgaccggggcgccaaggttatcgctaccgg
tttcgctgacctgggcttcgatgtggatatcggaccactgtttcaaacccccagagaagttgcccaacaagccgttgacgctgacgtacacg
ctgtaggcatctccactctcgccgccgggcataagactctcgtcccagagctgataaaggagcttaacagcctcggaagacccgacatcct
ggttatgtgcggtggagtgattccgccgcaggattacgaattcctcttcgaagtaggagtgtcaaacgtgttcggcccaggcactcggatac
ccaaggctgccgttcaggtgcttgacgacattgaaaaatgtctggagaagaagcaacaatctgtataa
SEQ ID NO: 13 is an optimized nucleotide sequence encoding human methylmalonyl-CoA
mutase (synMUT3)
atgttgagggctaaaaaccagctctttctgttgagtccacactaccttaggcaagtgaaggaatctagcggtagcaggctgatccagcagcg
cctgctgcaccagcagcagcccctgcaccctgagtgggctgcattggcaaagaaacaactgaagggtaaaaatcctgaagatctgatttgg
cacacaccggaggggatttccataaaacctctctactctaaacgcgatactatggatctgcccgaggaattgccaggagtgaaaccctttac
aagggggccctaccccactatgtacacgttcagaccctggactatacgccagtatgccggattttctaccgttgaggaatccaacaagttttat
aaggacaacatcaaagccgggcagcagggactgtcagtggcatttgatctcgccacccaccgcgggtacgactccgacaacccaagagt
ccgcggtgacgtcggcatggcaggggttgccattgacacagtagaggatactaaaattttgtttgatgggatccccctagagaagatgtccg
tgtctatgacgatgaacggcgcggtaatcccagtgcttgccaacttcatagtcacaggggaagagcagggcgtaccaaaggagaagctca
caggaacaatccaaaatgacattctgaaggaattcatggtgagaaatacttatatctttcctcccgagccctctatgaagattattgccgacat
ttttgaatacaccgcaaaacatatgcccaagttcaattccatatctattagtggataccacatgcaagaagctggggctgatgcaatacttgag
cttgcctacaccctggccgacggactggagtattctcgcactggcctgcaagccgggctgacaattgacgagttcgccccacgccttagcttct
tctggggcatcggcatgaatttctatatggagatcgcaaagatgagagcagggcggcgcttgtgggcccatctgatcgaaaagatgtttcag
cctaagaatagtaagagcctgctcctgcgggctcactgtcagacgtcaggctggagcctcacagagcaggatccttacaataacatcgtcc
ggactgctattgaggcgatggctgcagtattcggaggaacacaaagcctgcacactaattctttcgatgaggctttggggctccctaccgtga
agtcagccagaattgcaagaaacacccaaataatcatccaagaagaatcagggatcccaaaagttgccgacccctggggaggaagttata
tgatggagtgcctgaccaatgacgtctacgacgccgctttgaagctgattaacgagattgaagagatgggcggaatggccaaggcggtcg
ctgagggcattccgaaactgcgcatagaggagtgtgctgctcgcaggcaggccagaattgattccggttccgaagtgatcgtgggggttaa
taagtatcaactggaaaaagaggacgctgtcgaagtcctcgcaatcgataataccagcgttagaaaccgacaaattgagaagctgaaaaag
atcaaaagttcaagggaccaggccttggctgagcggtgtctcgccgcactgaccgaatgtgccgccagcggcgatggtaacatcctcgcc
ctcgctgtggacgcttccagagcccggtgcaccgtgggcgaaattacggacgcgctgaaaaaagtctttggcgaacacaaggccaatgat
agaatggtgagtggcgcctataggcaggagttcggcgagagtaaagaaataacatccgccatcaagagggtccacaaatttatggagcgg
gaaggacgcagacctagacttctcgtggccaaaatgggtcaggacggtcatgaccggggagccaaagtcatcgcaacgggcttcgccga
tttggggtttgacgtggatatcggtcccttgtttcaaacccccagggaggtggctcagcaggctgtggacgctgacgtccacgcagtgggca
tttctacactggcagccgggcacaagacgttggtgccagaactgatcaaagagttgaacagcctgggacgccctgacatcctggtaatgtg
cggtggggtaatccccccccaagactacgagttccttttcgaagtgggtgtttctaacgtgttcggacctggaacaagaatccctaaggcgg
cagtgcaggtgcttgacgatatcgagaagtgcctggagaaaaagcaacaatccgtttaa
SEQ ID NO: 14 is an optimized nucleotide sequence encoding human methylmalonyl-CoA
mutase (synMUT4)
atgcttcgcgccaagaaccaactgttcctgctgtccccccactacctccgacaagtcaaggagagctcgggaagccgcctgattcagcagc
ggctgctgcaccagcagcagcccctgcatccggaatgggcagcgttggcaaagaagcagctgaagggaaagaaccagaggacctgat
ctggcacaccccggagggaatctcgatcaagccactgtactccaaaagggacaccatggacttgcctgaagaacttccgggcgtgaagcc
ttttacccgggggccatacccaacaatgtacactttccgcccctggaccatcagacagtacgccggtttctccaccgtcgaagaatccaaca
agttctataaggacaacatcaaggccgggcagcagggactgagcgtcgcgtttgacctggcaacccatcgcggctacgactccgacaac
cacgcgtgcggggggacgtgggaatggccggagtggctatcgacaccgtggaggacaccaagattctcttcgacggaatcccgctgga
aaagatgtcggtgtccatgaccatgaatggcgccgtgatcccggtgctcgcgaacttcatcgtgacgggagaggaacagggagtgccga
aagagaagagaccgggactattcagaatgacatcctcaaggagttcatggtccgcaacacttacattttccctcctgaaccctcgatgaaga
tcatcgctgacatcttcgagtacaccgcgaagcacatgccgaagttcaactcgatctccatctcgggctaccacatgcaggaggccggggc
cgacgccattacgaactggcgtacactaggcggatggtaggaatactcacgcaccggactgcaggccggactgacaatcgacgagtt
cgccccgaggctgtccttcttctggggcattgggatgaacttctatatggaaatcgcgaagatgagagaggaaggcggctgtgggcgcac
ctgatcgagaagatgttccagcccaagaacagcaaaagccttctcctccgcgcccactgccaaacttccggctggtcactgaccgagcag
gatccgtacaacaacattgtccggactgccattgaggccatggccgctgtgttcggaggcactcagtccctccacactaactccttcgacga
ggccagggtagccgaccgtgaagtccgcccggatagccagaaatactcaaatcattatccaggaggaaagcggaatccccaaggtcg
ccgacccttggggaggatcttacatgatggagtgtttgaccaatgacgtctacgacgccgccagaagctcattaacgaaatcgaagagatg
ggcggaatggccaaggccgtggctgagggcatcccgaagctgagaatcgaggaatgcgccgcccggagacaggcccgcattgatagc
ggcagcgaggtcattgtgggcgtgaacaagtaccagcttgaaaaggaggacgccgtggaagtgctggcaatcgataacacctccgtgcg
caaccggcagatcgaaaagctcaagaagattaagtcctcacgggaccaggcactggcggagagatgcctcgccgcgctgaccgaatgc
gctgcctcgggagatggcaacattctggccaggcagtggacgcctctcgggctcggtgcactgtgggggagatcaccgacgccctcaa
gaaagtgttcggtgaacataaggccaacgaccggatggtgtccggagcgtaccgccaggaatttggcgaatcaaaggaaatcacgtccg
caatcaagagggtgcacaaattcatggaacgggagggcagacggcccagactgctcgtggctaaaatgggacaagatggtcacgaccg
cggcgccaaggtcatcgcgactggcttcgccgatctcggattcgacgtggacatcggacctagtttcaaactccccgggaagtggcccag
caggccgtggacgcggacgtgcatgccgtcgggatctcaaccaggcggccggccataagaccaggtgccggaactgatcaaggagc
tgaactcgctcggccgccccgacatcctcgtgatgtgtggcggagtgattccgccacaagactacgagttcctgttcgaagtcggggtgtcc
aacgtgttcggtcccggaaccagaatcccgaaggctgcggtccaagtgctggatgatattgagaagtgccttgagaaaaagcaacagtca
gtgtga
SEQ ID NO: 16 is a nucleotide sequence encoding a construct for expressing Mut in mice.
This is the murine sequence for LB-001. Components of the sequence include: ITR

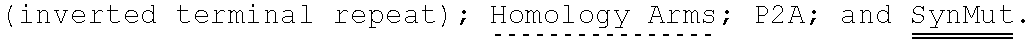

TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA

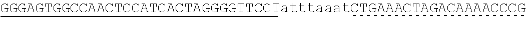

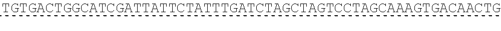

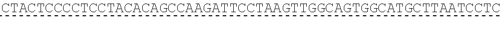

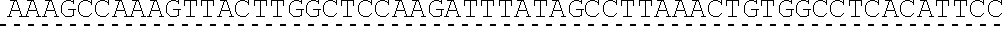

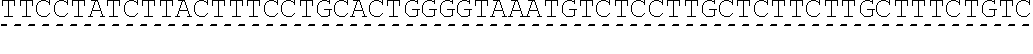

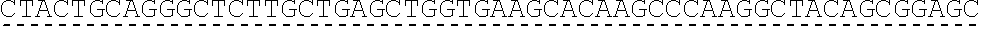

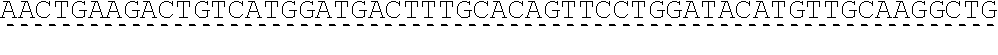

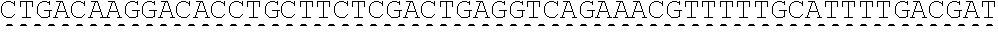

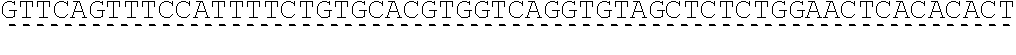

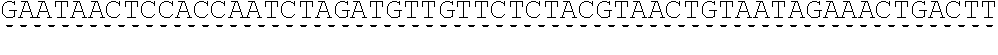

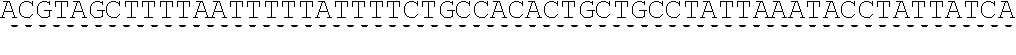

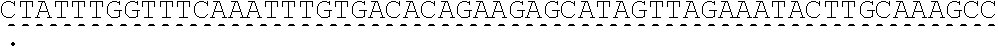

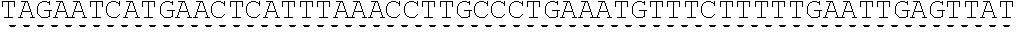

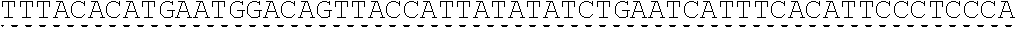

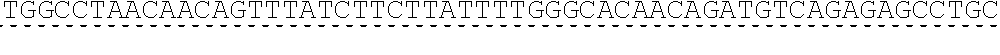

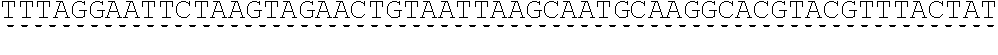

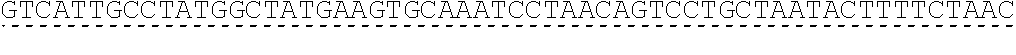

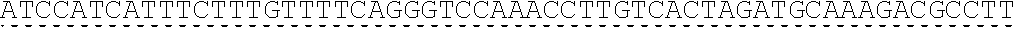

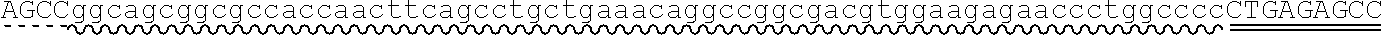

AAAAACCAGCTGTTCCTGCTGAGCCCCCACTATCTGAGACAGGTCAAAGAAAGTTC
CGGGAGTAGACTGATCCAGCAGAGACTGCTGCACCAGCAGCAGCCACTGCATCCTG
AGTGGGCCGCTCTGGCCAAGAAACAGCTGAAGGGCAAAAACCCAGAAGACCTGATC
TGGCACACTCCAGAGGGGATTTCAATCAAGCCCCTGTACAGCAAAAGGGACACTAT
GGATCTGCCAGAGGAACTGCCAGGAGTGAAGCCTTTCACCCGCGGACCTTACCCAA
CTATGTATACCTTTCGACCCTGGACAATTCGGCAGTACGCCGGCTTCAGTACTGTGG
AGGAATCAAACAAGTTTTATAAGGACAACATCAAGGCTGGACAGCAGGGCCTGAGT
GTGGCATTCGATCTGGCCACACATCGCGGCTATGACTCAGATAATCCCAGAGTCAGG
GGGGACGTGGGAATGGCAGGAGTCGCTATCGACACAGTGGAAGATACTAAGATTCT
GTTCGATGGAATCCCTCTGGAGAAAATGTCTGTGAGTATGACAATGAACGGCGCTGT
CATTCCCGTGCTGGCAAACTTCATCGTCACTGGCGAGGAACAGGGGGTGCCTAAGG
AAAAACTGACCGGCACAATTCAGAACGACATCCTGAAGGAGTTCATGGTGCGGAAT
ACTTACATTTTTCCCCCTGAACCATCCATGAAAATCATTGCCGATATCTTCGAGTACA
CCGCTAAGCACATGCCCAAGTTCAACTCAATTAGCATCTCCGGGTATCATATGCAGG
AAGCAGGAGCCGACGCTATTCTGGAGCTGGCTTACACCCTGGCAGATGGCCTGGAA
TATTCTCGAACCGGACTGCAGGCAGGCCTGACAATCGACGAGTTCGCTCCTAGACTG
AGTTTCTTTTGGGGAATTGGCATGAACTTTTACATGGAGATCGCCAAGATGAGGGCT
GGCCGGAGACTGTGGGCACACCTGATCGAGAAGATGTTCCAGCCTAAGAACTCTAA
GAGTCTGCTGCTGCGGGCCCATTGCCAGACATCCGGCTGGTCTCTGACTGAACAGGA
CCCATATAACAATATTGTCAGAACCGCAATCGAGGCAATGGCAGCCGTGTTCGGAG
GAACCCAGAGCCTGCACACAAACTCCTTTGATGAGGCCCTGGGGCTGCCTACCGTG
AAGTCTGCTAGGATTGCACGCAATACACAGATCATTATCCAGGAGGAATCCGGAAT
CCCAAAGGTGGCCGATCCCTGGGGAGGCTCTTACATGATGGAGTGCCTGACAAACG
ACGTGTATGATGCTGCACTGAAGCTGATTAATGAAATCGAGGAAATGGGGGGAATG
GCAAAGGCCGTGGCTGAGGGCATTCCAAAACTGAGGATCGAGGAATGTGCAGCTAG
GCGCCAGGCACGAATTGACTCAGGAAGCGAAGTGATCGTCGGGGTGAATAAGTACC
AGCTGGAGAAAGAAGACGCAGTCGAAGTGCTGGCCATCGATAACACAAGCGTGCGC
AATCGACAGATTGAGAAGCTGAAGAAAATCAAAAGCTCCCGCGATCAGGCACTGGC
CGAACGATGCCTGGCAGCCCTGACTGAGTGTGCTGCAAGCGGGGACGGAAACATTC
TGGCTCTGGCAGTCGATGCCTCCCGGGCTAGATGCACTGTGGGGGAAATCACCGAC
GCCCTGAAGAAAGTCTTCGGAGAGCACAAGGCCAATGATCGGATGGTGAGCGGCGC
TTATAGACAGGAGTTCGGGGAATCTAAAGAGATTACCAGTGCCATCAAGAGGGTGC
ACAAGTTCATGGAGAGAGAAGGGCGACGGCCCAGGCTGCTGGTGGCAAAGATGGG
ACAGGACGGACATGATCGCGGAGCAAAAGTCATTGCCACCGGGTTCGCTGACCTGG
GATTTGACGTGGATATCGGCCCTCTGTTCCAGACACCACGAGAGGTCGCACAGCAG
GCAGTCGACGCTGATGTGCACGCAGTCGGAGTGTCCACTCTGGCAGCTGGCCATAA
GACCCTGGTGCCTGAACTGATCAAAGAGCTGAACTCTCTGGGCAGACCAGACATCC
TGGTCATGTGCGGCGGCGTGATCCCACCCCAGGATTACGAATTCCTGTTTGAGGTCG
GGGTGAGCAACGTGTTCGGACCAGGAACCAGGATCCCTAAGGCCGCAGTGCAGGTC

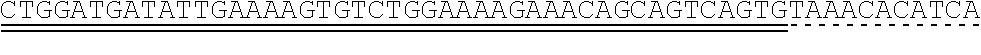

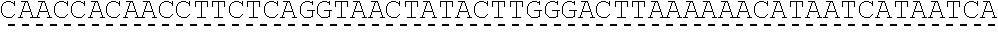

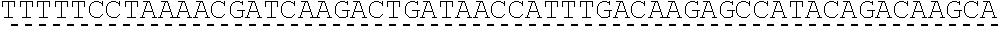

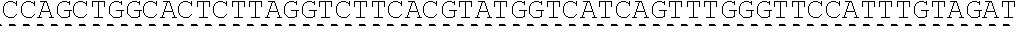

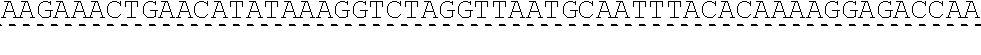

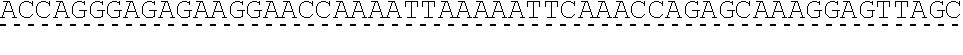

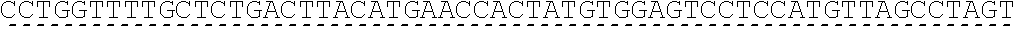

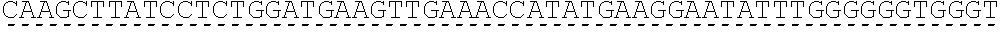

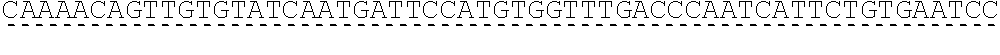

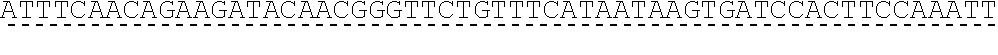

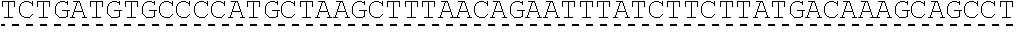

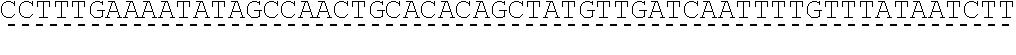

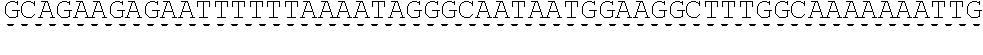

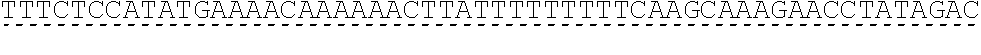

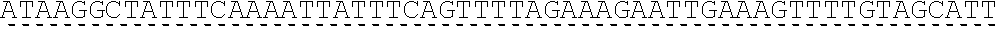

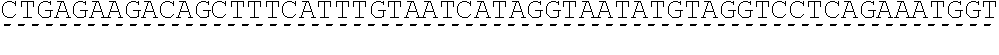

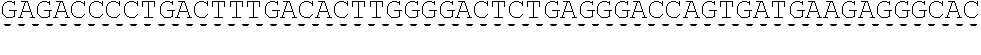

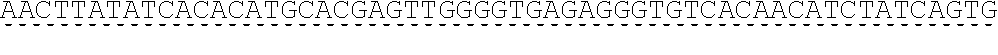

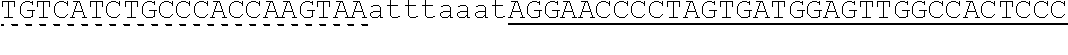

TCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAA

1. A method of integrating a transgene into the genome of at least a population of cells in a tissue in a subject, said method comprising
administering to a subject in which cells in the tissue fail to express a functional protein encoded by a gene product, a composition that delivers a transgene encoding the functional protein, wherein the composition comprises:
a polynucleotide cassette comprising
a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products upon integration into a target integration site in the genome of the cell;
a third nucleic acid sequence positioned 5′ to the polynucleotide and comprising a sequence that is substantially homologous to a genomic sequence 5′ of the target integration site in the genome of the cell; and
a fourth nucleic acid sequence positioned 3′ to the polynucleotide and comprising a sequence that is substantially homologous to a genomic sequence 3′ of the target integration site in the genome of the cell;
wherein, after administering the composition, the transgene is integrated into the genome of the population of cells.

2. The method of claim 1, wherein the integration does not comprise nuclease activity.

3. The method of claim 1, wherein the composition comprises a recombinant viral vector.

4. (canceled)

5. The method of claim 3, wherein the recombinant viral vector is or comprises a capsid protein comprising an amino acid sequence having at least 95% sequence identity with the amino acid sequence of LK03, AAV8, AAV-DJ; AAV-LK03; or AAVNP59.

6. The method of claim 1, wherein the transgene is or comprises a MUT transgene.

7. (canceled)

8. The method of claim 1, wherein the polynucleotide cassette does not comprise a promoter sequence.

9. (canceled)

10. The method of claim 1, wherein the target integration site is an albumin locus comprising an endogenous albumin promoter and an endogenous albumin gene.

11. (canceled)

12. The method of claim 10, wherein the tissue is the liver.

13. The method of claim 1, wherein the second nucleic acid sequence comprises:
a) a 2A peptide;
b) an internal ribosome entry site (IRES);
c) an N-terminal intein splicing region and C-terminal intein splicing region; or
d) a splice donor and a splice acceptor.

14.-15. (canceled)

16. The method of claim 6, wherein the MUT transgene is a wt human MUT; a codon optimized MUT; a synthetic MUT; a MUT variant; a MUT mutant, or a MUT fragment.

17.-33. (canceled)

34. A recombinant viral vector for integrating a transgene into a target integration site in the genome of a cell, comprising:
(i) a polynucleotide cassette comprising a first nucleic acid sequence and a second nucleic acid sequence, wherein the first nucleic acid sequence comprises a MUT transgene; and the second nucleic acid sequence is positioned 5′ or 3′ to the first nucleic acid sequence and promotes the production of two independent gene products upon integration into the target integration site in the genome of the cell;
(ii) a third nucleic acid sequence positioned 5′ to the polynucleotide cassette vector and comprising a sequence that is substantially homologous to a genomic sequence 5′ of the target integration site in the genome of the cell; and
(iii) a fourth nucleic acid sequence positioned 3′ of the polynucleotide cassette viral vector and comprising a sequence that is substantially homologous to a genomic sequence 3′ of the target integration site in the genome of the cell;
wherein the viral vector comprises an LK03 AAV capsid.

35. The recombinant viral vector of claim 34, wherein the third and fourth nucleic acids are independently between 800-1,200 nucleotides.

36.-38. (canceled)

39. The recombinant viral vector of claim 34, further comprising AAV2 ITR sequences.

40. The recombinant viral vector of claim 34, wherein the polynucleotide cassette does not comprise a promoter sequence.

41.-43. (canceled)

44. The recombinant viral vector of claim 34, wherein the two independent gene products are a MUT protein expressed from the MUT transgene and an endogenous albumin protein expressed from an endogenous albumin gene.

45. The recombinant viral vector of claim 34, wherein the cell is a liver cell.

46. The recombinant viral vector of claim 34, wherein the second nucleic acid sequence comprises:
a) a 2A peptide;
b) an internal ribosome entry site (IRES);
c) an N-terminal intein splicing region and a C-terminal intein splicing region; or
d) a splice donor and a splice acceptor.

47.-49. (canceled)

50. The recombinant viral vector of any one of claims 34-49, wherein the MUT transgene is a wt human MUT; a codon optimized MUT; a synthetic MUT; a MUT variant; a MUT mutant, or a MUT fragment.

51.-67. (canceled)

68. A recombinant viral vector for integrating a transgene into a target integration site in the genome of a cell, comprising:
(i) a polynucleotide cassette comprising a first nucleic acid sequence and a second nucleic acid sequence, wherein the first nucleic acid sequence comprises a MUT transgene; and the second nucleic acid sequence is positioned 5′ or 3′ to the first nucleic acid sequence and comprises a sequence encoding a P2A peptide;
(ii) a third nucleic acid sequence 1000 nt in length positioned 5′ to the polynucleotide cassette vector and comprising a sequence that is substantially homologous to a genomic sequence 5′ of an albumin gene in the genome of the cell; and
(iii) a fourth nucleic acid sequence 1000 nt in length positioned 3′ of the polynucleotide cassette vector and comprising a sequence that is substantially homologous to a genomic sequence 3′ of an albumin gene in the genome of the cell;
wherein the viral vector comprises an LK03 AAV capsid.

69. The recombinant viral vector of claim 68, wherein the vector comprises the nucleic acid sequence of SEQ ID NO. 15.


SEQUENCE LISTING (19 sequences)

SEQ ID NO: 1 - protein, 737 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLKTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 2 - DNA, 2215 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagtttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 3 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNRGLTVDTNGVYSEPRPIGTRYLTRNL

SEQ ID NO: 4 - protein, 738 aa, Artificial Sequence
  Description of Artificial Sequence Synthetic polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 5 - DNA, 2211 bp, Artificial Sequence
  Description of Artificial Sequence Synthetic polynucleotide
atggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaa

SEQ ID NO: 6 - protein, 736 aa, Artificial Sequence
  Description of Artificial Sequence Synthetic polypeptide
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 7 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggcaagacaggccagcagcccgctaaaaagagactcaattttggtcagactggcgactcagagtcagtcccagaccctcaacctctcggagaaccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcgacacacaaggcgttcttccaggcatggtctggcaggacagagatgtgtaccttcagggacccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKTGQQPAKKRLNFGQTGDSESVPDPQPLGEPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVDTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - DNA, 2253 bp, Artificial Sequence
  Description of Artificial Sequence Synthetic polynucleotide
atgctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaa

SEQ ID NO: 10 - protein, 750 aa, Homo sapiens
MLRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAEHCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGVSTLAAGHKTLVPELIKELNSLGRPDILVMCGGVIPPQDYEFLFEVGVSNVFGPGTRIPKAAVQVLDDIEKCLEKKQQSV

SEQ ID NO: 11 - DNA, 2253 bp, Homo sapiens
atgttaagagctaagaatcagctttttttactttcacctcattacctgaggcaggtaaaagaatcatcaggctccaggctcatacagcaacgacttctacaccagcaacagccccttcacccagaatgggctgccctggctaaaaagcagctgaaaggcaaaaacccagaagacctaatatggcacaccccggaagggatctctataaaacccttgtattccaagagagatactatggacttacctgaagaacttccaggagtgaagccattcacacgtggaccatatcctaccatgtatacctttaggccctggaccatccgccagtatgctggttttagtactgtggaagaaagcaataagttctataaggacaacattaaggctggtcagcagggattatcagttgcctttgatctggcgacacatcgtggctatgattcagacaaccctcgagttcgtggtgatgttggaatggctggagttgctattgacactgtggaagataccaaaattctttttgatggaattcctttagaaaaaatgtcagtttccatgactatgaatggagcagttattccagttcttgcaaattttatagtaactggagaagaacaaggtgtacctaaagagaagcttactggtaccatccaaaatgatatactaaaggaatttatggttcgaaatacatacatttttcctccagaaccatccatgaaaattattgctgacatatttgaatatacagcaaagcacatgccaaaatttaattcaatttcaattagtggataccatatgcaggaagcaggggctgatgccattctggagctggcctatactttagcagatggattggagtactctagaactggactccaggctggcctgacaattgatgaatttgcaccaaggttgtctttcttctggggaattggaatgaatttctatatggaaatagcaaagatgagagctggtagaagactctgggctcacttaatagagaaaatgtttcagcctaaaaactcaaaatctcttcttctaagagcacactgtcagacatctggatggtcacttactgagcaggatccctacaataatattgtccgtactgcaatagaagcaatggcagcagtatttggagggactcagtctttgcacacaaattcttttgatgaagctttgggtttgccaactgtgaaaagtgctcgaattgccaggaacacacaaatcatcattcaagaagaatctgggattcccaaagtggctgatccttggggaggttcttacatgatggaatgtctcacaaatgatgtttatgatgctgctttaaagctcattaatgaaattgaagaaatgggtggaatggccaaagctgtagctgagggaatacctaaacttcgaattgaagaatgtgctgcccgaagacaagctagaatagattctggttctgaagtaattgttggagtaaataagtaccagttggaaaaagaagacgctgtagaagttctggcaattgataatacttcagtgcgaaacaggcagattgaaaaacttaagaagatcaaatccagcagggatcaagctttggctgaacgttgtcttgctgcactaaccgaatgtgctgctagcggagatggaaatatcctggctcttgcagtggatgcatctcgggcaagatgtacagtgggagaaatcacagatgccctgaaaaaggtatttggtgaacataaagcgaatgatcgaatggtgagtggagcatatcgccaggaatttggagaaagtaaagagataacatctgctatcaagagggttcataaattcatggaacgtgaaggtcgcagacctcgtcttcttgtagcaaaaatgggacaagatggccatgacagaggagcaaaagttattgctacaggatttgctgatcttggttttgatgtggacataggccctcttttccagactcctcgtgaagtggcccagcaggctgtggatgcggatgtgcatgctgtgggcataagcaccctcgctgctggtcataaaaccctagttcctgaactcatcaaagaacttaactcccttggacggccagatattcttgtcatgtgtggaggggtgataccacctcaggattatgaatttctgtttgaagttggtgtttccaatgtatttggtcctgggactcgaattccaaaggctgccgttcaggtgcttgatgatattgagaagtgtttggaaaagaagcagcaatctgtataa

SEQ ID NO: 12 - DNA, 2253 bp, Artificial Sequence
  Description of Artificial Sequence Synthetic polynucleotide
atgctgcgagcgaaaaatcagctttttctgttgagcccacactacctgaggcaggttaaagaatccagcgggagccggctgattcagcagcgactgctccaccagcagcagcctttgcatcccgaatgggctgctttggcgaagaagcagctcaaggggaagaaccctgaagatcttatttggcacaccccagagggcatcagcatcaagcctttgtattccaaaagggacaccatggatctgcctgaagaattgcccggggtcaaaccattcacacgggggccatatccaaccatgtacaccttccggccatggactatcagacagtatgcaggctttagcactgtcgaggaatccaataagttctataaagacaatatcaaagctggccagcaaggtctgtccgtggcattcgatctggctacacatagaggttatgattctgacaatccaagagtacggggagacgtcggaatggcgggagttgccattgacacagtggaggacaccaagatacttttcgatgggattccattggagaaaatgtctgtgtcaatgacgatgaacggcgctgtgattcccgttttggcgaacttcatcgtcaccggggaagagcagggcgtcccgaaggaaaagctcaccgggacaatccaaaacgacattcttaaagaattcatggtgagaaatacctacatctttcctcctgagccttccatgaagatcatcgcggacatctttgaatacacggctaaacacatgcctaaatttaactcaatcagcataagcgggtaccacatgcaggaggccggcgctgacgctatacttgagctcgcatataccctggcagatggactggaatactcaaggaccgggctccaggctggactgacaatcgacgagtttgccccccgactcagttttttctggggtatcgggatgaatttctacatggagatagcgaagatgagggcgggcagacggctttgggcgcatctgatcgagaaaatgttccagcccaagaattcaaagagtctgctgctgagagcccactgccagacctcaggctggagcctgactgaacaggacccatacaacaacattgttagaaccgccatcgaggcgatggcagcggttttcggtgggacacagtcattgcacactaactcatttgacgaagccctcggtctgcctaccgtgaagtcagctcggatcgctaggaacacacagatcatcatccaggaggagagtggcatcccaaaagtcgccgatccttggggaggaagttacatgatggaatgcctcacgaatgacgtatacgatgccgcactcaagctgattaacgagatcgaggaaatgggaggcatggcaaaagctgtcgccgagggcattccaaagctgcgcatagaggagtgtgccgcccgaagacaggcccgcattgactccggctctgaggtgatagtgggcgttaataaatatcagctagagaaggaagacgccgtcgaagttctggcgatagataatacctctgtgcgaaatagacagattgagaaactgaagaagatcaagtcaagccgagaccaggccttggccgagaggtgtctggcagccctcactgagtgcgcggcatctggggacggcaacatattggcacttgccgtcgatgcctccagggcccgatgtacggtcggcgaaattaccgatgccctcaagaaggtttttggcgagcacaaggctaacgacaggatggttagtggagcatacagacaggagtttggcgaaagcaaggaaattacttccgcgattaaaagagtgcacaaattcatggaacgggagggtaggcgaccgaggctcctcgttgccaaaatgggtcaggacggccacgaccggggcgccaaggttatcgctaccggtttcgctgacctgggcttcgatgtggatatcggaccactgtttcaaacccccagagaagttgcccaacaagccgttgacgctgacgtacacgctgtaggcatctccactctcgccgccgggcataagactctcgtcccagagctgataaaggagcttaacagcctcggaagacccgacatcctggttatgtgcggtggagtgattccgccgcaggattacgaattcctcttcgaagtaggagtgtcaaacgtgttcggcccaggcactcggatacccaaggctgccgttcaggtgcttgacgacattgaaaaatgtctggagaagaagcaacaatctgtataa

SEQ ID NO: 13 - DNA, 2253 bp, Artificial Sequence
  Description of Artificial Sequence Synthetic polynucleotide
atgttgagggctaaaaaccagctctttctgttgagtccacactaccttaggcaagtgaaggaatctagcggtagcaggctgatccagcagcgcctgctgcaccagcagcagcccctgcaccctgagtgggctgcattggcaaagaaacaactgaagggtaaaaatcctgaagatctgatttggcacacaccggaggggatttccataaaacctctctactctaaacgcgatactatggatctgcccgaggaattgccaggagtgaaaccctttacaagggggccctaccccactatgtacacgttcagaccctggactatacgccagtatgccggattttctaccgttgaggaatccaacaagttttataaggacaacatcaaagccgggcagcagggactgtcagtggcatttgatctcgccacccaccgcgggtacgactccgacaacccaagagtccgcggtgacgtcggcatggcaggggttgccattgacacagtagaggatactaaaattttgtttgatgggatccccctagagaagatgtccgtgtctatgacgatgaacggcgcggtaatcccagtgcttgccaacttcatagtcacaggggaagagcagggcgtaccaaaggagaagctcacaggaacaatccaaaatgacattctgaaggaattcatggtgagaaatacttatatctttcctcccgagccctctatgaagattattgccgacatttttgaatacaccgcaaaacatatgcccaagttcaattccatatctattagtggataccacatgcaagaagctggggctgatgcaatacttgagcttgcctacaccctggccgacggactggagtattctcgcactggcctgcaagccgggctgacaattgacgagttcgccccacgccttagcttcttctggggcatcggcatgaatttctatatggagatcgcaaagatgagagcagggcggcgcttgtgggcccatctgatcgaaaagatgtttcagcctaagaatagtaagagcctgctcctgcgggctcactgtcagacgtcaggctggagcctcacagagcaggatccttacaataacatcgtccggactgctattgaggcgatggctgcagtattcggaggaacacaaagcctgcacactaattctttcgatgaggctttggggctccctaccgtgaagtcagccagaattgcaagaaacacccaaataatcatccaagaagaatcagggatcccaaaagttgccgacccctggggaggaagttatatgatggagtgcctgaccaatgacgtctacgacgccgctttgaagctgattaacgagattgaagagatgggcggaatggccaaggcggtcgctgagggcattccgaaactgcgcatagaggagtgtgctgctcgcaggcaggccagaattgattccggttccgaagtgatcgtgggggttaataagtatcaactggaaaaagaggacgctgtcgaagtcctcgcaatcgataataccagcgttagaaaccgacaaattgagaagctgaaaaagatcaaaagttcaagggaccaggccttggctgagcggtgtctcgccgcactgaccgaatgtgccgccagcggcgatggtaacatcctcgccctcgctgtggacgcttccagagcccggtgcaccgtgggcgaaattacggacgcgctgaaaaaagtctttggcgaacacaaggccaatgatagaatggtgagtggcgcctataggcaggagttcggcgagagtaaagaaataacatccgccatcaagagggtccacaaatttatggagcgggaaggacgcagacctagacttctcgtggccaaaatgggtcaggacggtcatgaccggggagccaaagtcatcgcaacgggcttcgccgatttggggtttgacgtggatatcggtcccttgtttcaaacccccagggaggtggctcagcaggctgtggacgctgacgtccacgcagtgggcatttctacactggcagccgggcacaagacgttggtgccagaactgatcaaagagttgaacagcctgggacgccctgacatcctggtaatgtgcggtggggtaatccccccccaagactacgagttccttttcgaagtgggtgtttctaacgtgttcggacctggaacaagaatccctaaggcggcagtgcaggtgcttgacgatatcgagaagtgcctggagaaaaagcaacaatccgtttaa

SEQ ID NO: 14 - DNA, 2253 bp, Artificial Sequence
  Description of Artificial Sequence Synthetic polynucleotide
atgcttcgcgccaagaaccaactgttcctgctgtccccccactacctccgacaagtcaaggagagctcgggaagccgcctgattcagcagcggctgctgcaccagcagcagcccctgcatccggaatgggcagcgttggcaaagaagcagctgaagggaaagaaccctgaggacctgatctggcacaccccggagggaatctcgatcaagccactgtactccaaaagggacaccatggacttgcctgaagaacttccgggcgtgaagccttttacccgggggccatacccaacaatgtacactttccgcccctggaccatcagacagtacgccggtttctccaccgtcgaagaatccaacaagttctataaggacaacatcaaggccgggcagcagggactgagcgtcgcgtttgacctggcaacccatcgcggctacgactccgacaaccctcgcgtgcggggggacgtgggaatggccggagtggctatcgacaccgtggaggacaccaagattctcttcgacggaatcccgctggaaaagatgtcggtgtccatgaccatgaatggcgccgtgatcccggtgctcgcgaacttcatcgtgacgggagaggaacagggagtgccgaaagagaagctgaccgggactattcagaatgacatcctcaaggagttcatggtccgcaacacttacattttccctcctgaaccctcgatgaagatcatcgctgacatcttcgagtacaccgcgaagcacatgccgaagttcaactcgatctccatctcgggctaccacatgcaggaggccggggccgacgccattctcgaactggcgtacactctggcggatggtctggaatactcacgcaccggactgcaggccggactgacaatcgacgagttcgccccgaggctgtccttcttctggggcattgggatgaacttctatatggaaatcgcgaagatgagagctggaaggcggctgtgggcgcacctgatcgagaagatgttccagcccaagaacagcaaaagccttctcctccgcgcccactgccaaacttccggctggtcactgaccgagcaggatccgtacaacaacattgtccggactgccattgaggccatggccgctgtgttcggaggcactcagtccctccacactaactccttcgacgaggccctgggtctgccgaccgtgaagtccgcccggatagccagaaatactcaaatcattatccaggaggaaagcggaatccccaaggtcgccgacccttggggaggatcttacatgatggagtgtttgaccaatgacgtctacgacgccgccctgaagctcattaacgaaatcgaagagatgggcggaatggccaaggccgtggctgagggcatcccgaagctgagaatcgaggaatgcgccgcccggagacaggcccgcattgatagcggcagcgaggtcattgtgggcgtgaacaagtaccagcttgaaaaggaggacgccgtggaagtgctggcaatcgataacacctccgtgcgcaaccggcagatcgaaaagctcaagaagattaagtcctcacgggaccaggcactggcggagagatgcctcgccgcgctgaccgaatgcgctgcctcgggagatggcaacattctggccctggcagtggacgcctctcgggctcggtgcactgtgggggagatcaccgacgccctcaagaaagtgttcggtgaacataaggccaacgaccggatggtgtccggagcgtaccgccaggaatttggcgaatcaaaggaaatcacgtccgcaatcaagagggtgcacaaattcatggaacgggagggcagacggcccagactgctcgtggctaaaatgggacaagatggtcacgaccgcggcgccaaggtcatcgcgactggcttcgccgatctcggattcgacgtggacatcggacctctgtttcaaactccccgggaagtggcccagcaggccgtggacgcggacgtgcatgccgtcgggatctcaaccctggcggccggccataagaccctggtgccggaactgatcaaggagctgaactcgctcggccgccccgacatcctcgtgatgtgtggcggagtgattccgccacaagactacgagttcctgttcgaagtcggggtgtccaacgtgttcggtcccggaaccagaatcccgaaggctgcggtccaagtgctggatgatattgagaagtgccttgagaaaaagcaacagtcagtgtga

SEQ ID NO: 15 - DNA, 4615 bp, Artificial Sequence
  Description of Artificial Sequence Synthetic polynucleotide
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagcttgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggcagcggcgccaccaacttcagcctgctgaaacaggccggcgacgtggaagagaaccctggccccctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgtacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 16 - DNA, 4619 bp, Mus sp.
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatttaaatctgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggcagcggcgccaccaacttcagcctgctgaaacaggccggcgacgtggaagagaaccctggccccctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatcaattttgtttataatcttgcagaagagaattttttaaaatagggcaataatggaaggctttggcaaaaaaattgtttctccatatgaaaacaaaaaacttatttttttattcaagcaaagaacctatagacataaggctatttcaaaattatttcagttttagaaagaattgaaagttttgtagcattctgagaagacagctttcatttgtaatcataggtaatatgtaggtcctcagaaatggtgagacccctgactttgacacttggggactctgagggaccagtgatgaagagggcacaacttatatcacacatgcacgagttggggtgagagggtgtcacaacatctatcagtgtgtcatctgcccaccaagtaaatttaaataggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 17 - DNA, 22 bp, Artificial Sequence
  Description of Artificial Sequence Synthetic primer
acattcaccttccatgcagata

SEQ ID NO: 18 - DNA, 19 bp, Artificial Sequence
  Description of Artificial Sequence Synthetic primer
tcagcaggctgaaattggt

SEQ ID NO: 19 - DNA, 247 bp, Artificial Sequence
  Description of Artificial Sequence Synthetic polynucleotide
  modified_base (1)..(12): a, c, t, g, unknown or other
  misc_feature (1)..(12): n is a, c, g, or t
  modified_base (15)..(16): a, c, t, g, unknown or other
  misc_feature (15)..(16): n is a, c, g, or t
  modified_base (20)..(20): a, c, t, g, unknown or other
  misc_feature (20)..(20): n is a, c, g, or t
  modified_base (22)..(22): a, c, t, g, unknown or other
  misc_feature (22)..(22): n is a, c, g, or t
  modified_base (24)..(24): a, c, t, g, unknown or other
  misc_feature (24)..(24): n is a, c, g, or t
  modified_base (83)..(83): a, c, t, g, unknown or other
  misc_feature (83)..(83): n is a, c, g, or t
  modified_base (234)..(234): a, c, t, g, unknown or other
  misc_feature (234)..(234): n is a, c, g, or t
  modified_base (243)..(243): a, c, t, g, unknown or other
  misc_feature (243)..(243): n is a, c, g, or t
  modified_base (245)..(246): a, c, t, g, unknown or other
  misc_feature (245)..(246): n is a, c, g, or t
nnnnnnnnnnnngannaganananaatcaagaaacaaactgcacttgttgagcttgtgaaacacaagcccaaggcaacaaaanagcaactgaaagctgttatggatgatttcgcagcttttgtagagaagtgctgcaaggctgacgataaggagacctgctttgccgaggagggtaaaaaacttgttgctgcaagtcaagctgccttaggcttaggcagcggcgccaccaattnagcctgctnanna